(19) United States
(12) Patent Application Publication
(10) Pub. No.: US 2025/0339528 A1
(43) Pub. Date: Nov. 6, 2025

(54) SELECTION OF OPTIMAL CELL DONORS AND METHODS AND COMPOSITIONS FOR ENHANCED EXPANSION AND CYTOTOXICITY OF DONOR CELLS

(71) Applicant: NKARTA, INC., South San Francisco, CA (US)

(72) Inventors: James Barnaby TRAGER, Albany, CA (US); Alexandra Leida Liana LAZETIC, San Jose, CA (US); Ivan CHAN, Millbrae, CA (US); Michael WHANG, San Francisco, CA (US); Ming-Hong XIE, Foster City, CA (US); Hadia LEMAR, Tracy, CA (US); Anmol VOHRA, San Francisco, CA (US); Ralph BRANDENBERGER, Palo Alto, CA (US)

(21) Appl. No.: 18/291,826

(22) PCT Filed: Jul. 26, 2022
(86) PCT No.: PCT/US2022/074164

§ 371 Date: Jan. 24, 2024

Related U.S. Application Data
(60) Provisional application No. 63/203,703, filed on Jul. 28, 2021.
(60) Provisional application No. 63/262,544, filed on Oct. 14, 2021.

(51) Int. Cl. A61K 40/15; A61K 40/31; A61K 40/42; C07K 14/725; C07K 16/28; C12N 5/0783

(52) U.S. Cl. CPC A61K 40/15; A61K 40/31; A61K 40/4211; C07K 14/7051; C07K 16/2803; C12N 5/0646; C12N 2501/2312; C12N 2501/2318; C12N 2502/00

(57) ABSTRACT

Several embodiments disclosed herein relate to methods and compositions for enhanced expansion of NK cells in culture, through the repeated co-culturing of NK cells with feeder cells and the selective use of stimulatory interleukins. In several embodiments, the methods utilize one or more soluble interleukins as culture media supplements at one or more time points during expansion of the NK cell, or other immune cell, which results in a highly expanded and highly cytotoxic population of cells, for use in, for example allogeneic cellular immunotherapy.

### CROSS REFERENCE TO RELATED APPLICATIONS

This application claims priority to U.S. Provisional Patent Application No. 63/203,703, filed Jul. 28, 2021 and U.S. Provisional Patent Application No. 63/262,544, filed Oct. 14, 2021, the entire contents of each of which is incorporated by reference herein.

### FIELD

Some embodiments of the methods and compositions disclosed herein relate to identification of donors of immune cells, such as Natural Killer (NK) cells and/or T cells, that exhibit enhanced capacity for expansion in culture and/or enhanced cytotoxicity against target tumor cells after being engineered to express, for example anti-tumor marker directed chimeric antigen receptors.

### BACKGROUND

The use of engineered cells for cellular immunotherapy allows for treatment of cancers or other diseases by leveraging various aspects of the immune system to target and destroy diseased or damaged cells. Such therapies require engineered cells in numbers sufficient for therapeutically relevant doses.

### INCORPORATION BY REFERENCE OF MATERIAL IN ASCII TEXT FILE

This application incorporates by reference the Sequence Listing contained in the following ASCII text file being submitted concurrently herewith: File name: NKT080WO_ST26.xml; created Jul. 25, 2022, 173,845 bytes in size.

### SUMMARY

In several embodiments, there are provided various methods for enhancing the expansion of immune cells for use in cellular immunotherapy. For example, in several embodiments, there is provided a method in which immune cells are co-cultured with a feeder cell line in a media supplemented with one or more soluble cytokines, the cytokines being added to the media at least once during the co-culture. In several embodiments, the immune cells are NK cells. In several embodiments, the expanded NK cells are unexpectedly amenable to cellular engineering, such as engineering the cells to express a chimeric receptor (for example, for use in cancer immunotherapy). In several embodiments, the NK cells (or other immune cells) co-cultured with a soluble interleukin-supplemented media express such chimeric receptors more robustly than NK cells not subject to the co-cultured in a soluble interleukin-supplemented media. Further, in several embodiments, the engineered NK cells exhibit an unexpectedly enhanced cytotoxicity.

In several embodiments, there is provided a method for enhancing the expansion of natural killer cells for use in immunotherapy, comprising co-culturing, for a first time, in a culture media supplemented with at least soluble interleukin 12 (IL12) and soluble interleukin 18 (IL18), a population of natural killer (NK) cells with a first batch of a feeder cell population, co-culturing, in a culture media, NK cells from the first co-culturing with a second batch of the feeder cell population, thereby generating a second co-culturing, co-culturing, in a culture media, NK cells from the second co-culturing with a third batch of the feeder cell population, thereby generating a third co-culturing, co-culturing, in the culture media, NK cells from the third co-culturing with a fourth batch of the feeder cell population, thereby generating a fourth co-culturing, and co-culturing, for a fifth time, in a culture media again supplemented with at least soluble IL12 and soluble IL18, NK cells from the fourth co-culturing with a fifth batch of the feeder cell population, thereby generating a fifth co-culturing, and resulting in a population of expanded NK cells

In several embodiments, the feeder cell population comprises cells engineered to express 4-1BBL and membrane-bound interleukin-15 (mbIL15). In several embodiments, a ratio of NK cells to feeder cells at each co-culturing ranges from about 1:2 to about 1:10. In several embodiments, the ratio of NK cells to feeder cells at each co-culturing ranges is about 1:3 to about 1:5. Other ratios are used in other embodiments, such as about 1:1, 1:4, 1:20, 1:50, 50:1, 25:1, 15:1, 10:1, 2:1 etc.

In several embodiments, the IL12 is present in the supplemented media at a concentration ranging from about 0.01 ng/ml to about 10 ng/ml (or at an equivalent concentration using other units of concentration, e.g., IU/mL). In several embodiments, the IL18 is present in the supplemented media at a concentration ranging from about 10 ng/mL to about 30 ng/ml (or at an equivalent concentration using other units of concentration, e.g., IU/mL). In several embodiments, one or more of the co-culturings employs media supplemented with soluble IL2. In several embodiments, the IL2 is present in the supplemented media at a concentration ranging from about 25 to about 50 units/mL (or at an equivalent concentration using other units of concentration, e.g., ng/ml). In several embodiments, the IL2 is present in the supplemented media for at least the first and the fifth co-culturing.

In several embodiments, the NK cells are frozen (e.g., cryopreserved) after a given co-culturing and thawed prior to the subsequent co-culturing. In several embodiments, the NK cells are frozen at least two times between the first and the fifth co-culturing.

In several embodiments, the methods further comprise genetically modification (e.g., gene editing) the NK cells to reduce or eliminate expression of at least one endogenous gene or protein expressed as compared to a non-modified NK cell, wherein the genetic modification is performed prior to the first or second co-culturing. In several embodiments, the genetic modification comprises a disruption of a gene encoding CISH, thereby resulting in reduced or eliminated CIS expression by the NK cell. Other genes disclosed herein may also be edited, alone, or in combination with CISH.

In several embodiments, the methods further comprise engineering the NK cells express a chimeric antigen receptor that is directed against a tumor target and promotes cytotoxic activity against a tumor cell expressing the tumor target. In several embodiments, the tumor target is selected from a ligand for the NKG2D receptor, CD19, CD70, BCMA, or CD38. In several embodiments, the engineering of the NK cells is concurrent or after the genetic editing. In several embodiments, the population of NK cells is derived from a peripheral blood sample collected from a donor. In several embodiments, the NK cells comprise KIR-educated NK cells. In several embodiments, the population of NK cells is derived from a cord blood sample. In several embodiments, the cord blood cells show limited to no signs of KIR education.

In some embodiments, there is provided a population of NK cells, wherein the NK cells were expanded according to methods disclosed herein. Also provided for herein are uses of populations of NK cells expanded and/or selected according to embodiments disclosed herein for the treatment of cancer. Additionally provided for herein are uses of populations of NK cells expanded and/or selected for according to embodiments disclosed herein in the preparation of a medicament for the treatment of cancer. Also provided for herein are methods of treating cancer comprising administering to a subject in need thereof a therapeutically effective amount of NK cells, wherein the NK cells were expanded according to methods disclosed herein.

In several embodiments, there is provided a population of expanded immune cells for use in immunotherapy, comprising a population immune cells that were expanded in culture, wherein the immune cells express a chimeric antigen receptor that is directed against a tumor target, and wherein the immune cells are optionally genetically edited to reduce or eliminate expression of at least one gene endogenous to the immune cell, wherein the population of immune cells were expanded by a process comprising co-culturing, for a first time, in a culture media, a population of immune cells with a first batch of a feeder cell population, wherein the feeder cell population comprises cells engineered to express 4-1BBL and membrane-bound interleukin-15 (mbIL15), wherein the culture media is supplemented with at least soluble interleukin 12 (IL12) and soluble interleukin 18 (IL18), co-culturing, in a culture media, immune cells from the first co-culturing with a second batch of the feeder cell population, co-culturing, in a culture media, immune cells from the second co-culturing with a third batch of the feeder cell population, co-culturing, in a culture media, immune cells from the third co-culturing with a fourth batch of the feeder cell population, co-culturing, for a fifth time, in a culture media, immune cells from the fourth co-culturing with a fifth batch of the feeder cell population, wherein the culture media is supplemented with at least soluble IL12 and soluble IL18, and wherein a population of expanded immune cells results from the plurality of co-culturings. In several embodiments, the immune cells are NK cells. In several embodiments, the NK cells are obtained from a peripheral blood sample. In several embodiments, the NK cells are obtained from a cord blood sample. In several embodiments, the immune cells are edited to reduce or eliminate expression of CISH. In several embodiments, the immune cells are engineered to express a CAR, wherein the CAR targets a ligand of the NKG2D receptor, CD19, CD70, BCMA, or CD38.

In several embodiments, there is also provided a population of expanded immune cells for use in immunotherapy, comprising a population immune cells that were expanded in culture, wherein the immune cells express aKIR and iKIR receptors and wherein the ratio of aKIR to iKIR expression prior to expansion was at least about 3. In several embodiments, the population of immune cells have been engineered to express a chimeric antigen receptor that is directed against a tumor target, and the immune cells are optionally genetically edited to reduce or eliminate expression of at least one gene endogenous to the immune cell. In several embodiments, the population of immune cells were expanded by a process comprising co-culturing, for a first time, in a culture media, a population of immune cells with a first batch of a feeder cell population, wherein the feeder cell population comprises cells engineered to express 4-1BBL and membrane-bound interleukin-15 (mbIL15) and wherein the culture media is supplemented with at least soluble interleukin 12 (IL12) and soluble interleukin 18 (IL18), co-culturing, a plurality of times, in a culture media, immune cells from a prior co-culturing with an additional batch of the feeder cell population, to generate a further expanded immune cell population, and co-culturing, for a final time, in the culture media, at least a portion of the further expanded immune cells with an additional batch of the feeder cell population, wherein the culture media is supplemented with at least soluble IL12 and soluble IL18 during the final co-culturing, and wherein a population of expanded immune cells results from the co-culturings, the expanded population exhibiting enhanced cytotoxicity and/or persistence as compared to a non-expanded population of immune cells. In several embodiments, the immune cells are NK cells. In several embodiments, the immune cells are edited to reduce or eliminate expression of CISH. In several embodiments, the population of expanded immune cells are engineered to express a CAR targeting a tumor marker, wherein the CAR targets a ligand of the NKG2D receptor, CD19, CD38, BCMA or CD70. Additionally provided for herein is a method for treating a cancer, comprising administering to a subject in need thereof a therapeutically effective amount of the population of expanded immune cells according to embodiments disclosed herein. Further provided is a use of the population of expanded immune cells according to embodiments disclosed herein for the preparation of a medicament for the treatment of cancer. Additionally, provided is a use of the population of expanded immune cells according to embodiments disclosed herein for the treatment of cancer.

In several embodiments, there is also provided a method for treating cancer comprising administering to a subject a population NK cells that were expanded in culture, wherein the NK cells express a chimeric antigen receptor that is directed against a tumor target, and wherein the NK cells express reduced amounts of CISH as compared to a native NK cell, wherein the population of NK cells were expanded by a process comprising co-culturing, for a first time, in a culture media, a population of NK cells with a first batch of a feeder cell population, wherein the feeder cell population comprises cells engineered to express 4-1BBL and membrane-bound interleukin-15 (mbIL15), wherein the culture media is supplemented with at least soluble interleukin 12 (IL12) and soluble interleukin 18 (IL18), co-culturing, a plurality of times, in a culture media, NK cells from a prior co-culturing with an additional batch of the feeder cell population, to generate a further expanded NK cell population, co-culturing, for a final time, in a culture media, at least a portion of the further expanded NK cells with an additional batch of the feeder cell population, wherein the culture media is supplemented with at least soluble IL12 and soluble IL18.

Additionally provided is a method for enhancing the expansion of natural killer cells for use in immunotherapy, comprising co-culturing, for a first time, in a culture media, a population of natural killer (NK) cells with a first batch of a feeder cell population, wherein the feeder cell population comprises cells engineered to express 4-1BBL and membrane-bound interleukin-15 (mbIL15), wherein the culture media is supplemented with at least soluble interleukin 12 (IL12) and soluble interleukin 18 (IL18), wherein a population of expanded NK cells results from the first co-culturing, co-culturing, for a second time, in a culture media, the expanded NK cells with a second batch of the feeder cell population, wherein a population of further expanded NK cells results from the second co-culturing, co-culturing, for at least a third time, in a culture media, the further expanded NK cells with a third batch of the feeder cell population, wherein a population of additionally further expanded NK cells results from the at least a third co-culturing; and co-culturing, for at least one additional time, in a culture media optionally supplemented with at least soluble IL12 and soluble IL18, the additionally further expanded NK cells from the at least a third co-culturing with an additional batch of a feeder cell population, wherein a population of finally expanded NK cells results from the at least one additional co-culturing, thereby resulting in enhanced NK cell expansion.

In several embodiments, there is provided a method for identifying a preferred donor of immune cells for immunotherapy, comprising obtaining a blood sample comprising immune cells from a candidate donor, detecting an expression level of at least one activating Killer Cell Ig-Like Receptor (aKIR), detecting an expression level of at least one inhibitory Killer Cell Ig-Like Receptor (iKIR), calculating a ratio of the expression level of the at least one aKIR and the at least one iKIR, categorizing the candidate donor as a preferred donor if the ratio of aKIR to iKIR exceeds a threshold value, wherein the threshold value is above about 3, and treating a subject in need of immunotherapy with immune cells expanded from the preferred donor. In several embodiments, the method further comprises assessing the ability of the immune cells from the candidate donor to be expanded in culture prior to said categorizing. In several embodiments, the method further comprises assessing the ability of the immune cells from the candidate donor to exert cytotoxic effects on a target tumor cell prior to said categorizing. In several embodiments, the method further comprises assessing the cytomegalovirus (CMV) status of the immune cells from the candidate donor prior to said categorizing. In several embodiments, the method further comprises detecting the degree of Human Leukocyte Antigen (HLA) mismatch between immune cells from the candidate donor and a target tumor cell by determining the number of iKIR triggered by tumor HLA. In several embodiments, the immune cells comprise natural killer (NK) cells, wherein the immune cells are derived from a peripheral blood sample. In several embodiments, the immune cells are derived from a cord blood sample.

In several embodiments, there is also provided a method for enhancing the expansion of natural killer cells for use in immunotherapy, comprising obtaining a population of natural killer (NK) cells from a preferred donor, wherein the NK cells from the preferred donor have a ratio of aKIR:iKIR expression of at least about 3, co-culturing, for a first time, in a culture media, the NK cells from the preferred donor with a first batch of a feeder cell population, wherein the feeder cell population comprises cells engineered to express 4-1BBL and membrane-bound interleukin-15 (mbIL15), wherein the culture media is supplemented with at least soluble interleukin 12 (IL12) and soluble interleukin 18 (IL18), wherein a population of expanded NK cells results from the first co-culturing, co-culturing, for a second time, in a culture media, the expanded NK cells with a second batch of the feeder cell population, wherein a population of further expanded NK cells results from the second co-culturing, co-culturing, for at least a third time, in a culture media, the further expanded NK cells with a third batch of the feeder cell population, wherein a population of additionally further expanded NK cells results from the at least a third co-culturing, and co-culturing, for at least one additional time, in a culture media supplemented with at least soluble IL12 and soluble IL18, the additionally further expanded NK cells from the at least a third co-culturing with an additional batch of a feeder cell population, wherein a population of finally expanded NK cells results from the at least one additional co-culturing, thereby resulting in enhanced NK cell expansion.

Additionally provided is a use of NK cells expanded by the methods disclosed herein or selected from a donor identified by the methods disclosed herein for the preparation of a medicament for the treatment of cancer. Also provided is a use of NK cells expanded by the methods disclosed herein or selected from a donor identified by disclosed herein for the treatment of cancer.

In several embodiments, there is provided a method for identifying a preferred donor of immune cells for immunotherapy, comprising obtaining a blood sample comprising immune cells from a candidate donor, detecting an expression level of at least one activating Killer Cell Ig-Like Receptor (aKIR), and categorizing the candidate donor as a preferred donor based on the detected aKIR expression.

In several embodiments, there is also provided an additional method for identifying a preferred donor of immune cells for immunotherapy, comprising obtaining a blood sample comprising immune cells from a candidate donor, detecting an expression level of at least one aKIR, detecting an expression level of at least one inhibitory Killer Cell Ig-Like Receptor (iKIR), calculating a ratio of the expression level of the at least one aKIR and the at least one iKIR, and categorizing the candidate donor as a preferred donor if the ratio of aKIR to iKIR exceeds a threshold value. In several embodiments, the threshold value is above about 3. In some embodiments, the threshold is at least about 4, 5, or 6. In several embodiments, the method further comprises treating a subject in need of immunotherapy with immune cells expanded from the preferred donor.

In several embodiments, the methods further comprise assessing the ability of the immune cells from the candidate donor to be expanded in culture prior to said categorizing. In several embodiments, the methods further comprise assessing the ability of the immune cells from the candidate donor to exert cytotoxic effects on a target tumor cell prior to said categorizing. In several embodiments, the methods further comprise assessing the cytomegalovirus (CMV) status of the immune cells from the candidate donor prior to said categorizing. In several embodiments, the methods further comprise detecting the degree of Human Leukocyte Antigen (HLA) mismatch between immune cells from the candidate donor and a target tumor cell by determining the number of iKIR triggered by tumor HLA.

In several embodiments, the immune cells comprise natural killer (NK) cells. In several embodiments, the immune cells comprise T cells. In several embodiments, the immune cells comprise combinations of NK cells and T cells.

In several embodiments, there is provided a method for enhancing the expansion of natural killer cells for use in immunotherapy, the method comprising obtaining a population of natural killer (NK) cells from a preferred donor, wherein the NK cells from the preferred donor have a ratio of aKIR:iKIR expression of at least about 3, co-culturing, for a first time, in a culture media, the NK cells from the preferred donor with a first batch of a feeder cell population, wherein the feeder cell population comprises cells engineered to express 4-1BBL and membrane-bound interleukin-15 (mbIL15), wherein the culture media is supplemented with at least soluble interleukin 12 (IL12) and soluble interleukin 18 (IL18), wherein a population of expanded NK cells results from the first co-culturing, co-culturing, for a second time, in the culture media, the expanded NK cells with a second batch of the feeder cell population, wherein a population of further expanded NK cells results from the second co-culturing, co-culturing, for at least a third time, in the culture media, the further expanded NK cells with a third batch of the feeder cell population, wherein a population of additionally further expanded NK cells results from the at least a third co-culturing; and co-culturing, for at least one additional time, in the culture media supplemented with at least soluble IL12 and soluble IL18, the additionally further expanded NK cells from the at least a third co-culturing with an additional batch of a feeder cell population, wherein a population of finally expanded NK cells results from the at least one additional co-culturing, thereby resulting in enhanced NK cell expansion. In several embodiments, advantageously, these methods result in at least several million-fold expansion of the NK cells, with substantially maintained genetic stability, and maintained, if not enhanced, cytotoxicity and persistence of the NK cells.

Additionally provided are methods for enhancing the expansion of natural killer cells for use in immunotherapy, comprising, consisting of, or consisting essentially of co-culturing, for a first time, in a culture media, a population of natural killer (NK) cells with a first batch of a feeder cell population, wherein the feeder cell population comprises cells engineered to express 4-1BBL and membrane-bound interleukin-15 (mbIL15), wherein the culture media is supplemented with at least soluble interleukin 12 (IL12) and soluble interleukin 18 (IL18), wherein a population of expanded NK cells results from the first co-culturing, co-culturing, for a second time, in the culture media, the expanded NK cells with a second batch of the feeder cell population, wherein a population of further expanded NK cells results from the second co-culturing, co-culturing, for at least a third time, in the culture media, the further expanded NK cells with a third batch of the feeder cell population, wherein a population of additionally further expanded NK cells results from the at least a third co-culturing, and co-culturing, for at least one additional time, in the culture media supplemented with at least soluble IL12 and soluble IL18, the additionally further expanded NK cells from the at least a third co-culturing with an additional batch of a feeder cell population, wherein a population of finally expanded NK cells results from the at least one additional co-culturing, thereby resulting in enhanced NK cell expansion. In several embodiments, advantageously, these methods result in at least several million-fold expansion of the NK cells, with substantially maintained genetic stability, and maintained, if not enhanced, cytotoxicity and persistence of the NK cells.

In several embodiments, there is also provided a method for enhancing the expansion of natural killer cells for use in immunotherapy, comprising, consisting of, or consisting essentially of co-culturing, for a first time, in a culture media, a population of natural killer (NK) cells with a first batch of a feeder cell population, wherein the feeder cell population comprises cells engineered to express 4-1BBL and membrane-bound interleukin-15 (mbIL15), wherein the culture media is supplemented with at least soluble interleukin 12 (IL12) and soluble interleukin 18 (IL18), co-culturing, in the culture media, NK cells from the first co-culturing with a second batch of the feeder cell population, co-culturing, in the culture media, NK cells from the second co-culturing with a third batch of the feeder cell population, co-culturing, in the culture media, NK cells from the third co-culturing with a fourth batch of the feeder cell population, co-culturing, for a fifth time, in the culture media, NK cells from the fourth co-culturing with a fifth batch of the feeder cell population, wherein the culture media is supplemented with at least soluble IL12 and soluble IL18, and wherein a population of expanded NK cells results from the plurality of co-culturings, thereby resulting in enhanced NK cell expansion. In several embodiments, advantageously, these methods result in at least several million-fold expansion of the NK cells, with substantially maintained genetic stability, and maintained, if not enhanced, cytotoxicity and persistence of the NK cells.

In several embodiments, the ratio of NK cells to feeder cells at the first co-culturing ranges from about 1:1 to about 1:10. In several embodiments, the ratio of NK cells to feeder cells at the first co-culturing ranges from about 1:2 to about 1:10. In several embodiments, the ratio of NK cells to feeder cells at the first co-culturing ranges is about 1:3 to about 1:5. In several embodiments, the ratio of NK cells to feeder cells is about 1:3. In several embodiments, the IL12 is present in the supplemented media at a concentration ranging from about 0.005 ng/ml to about 30 ng/ml, including about 0.01 ng/ml to about 10 ng/ml. In several embodiments, the IL18 is present in the supplemented media at a concentration ranging from about 0.005 ng/ml to about 30 ng/ml, including about 10 ng/ml to about 30 ng/ml. In several embodiments, the media is further supplemented with soluble IL2 for at least one co-culturing. In several embodiments, the IL2 is present in the supplemented media at a concentration ranging from about 5 to about 100 units/mL, including about 25 to about 50 units/mL. In several embodiments, the IL2 is present in the supplemented media for at least the first and a fifth co-culturing.

Depending on the embodiment, the cells are optionally frozen after a given co-culturing and thawed prior to the subsequent co-culturing. In several embodiments, the NK cells are frozen at least two times between the first and a fifth co-culturing.

In several embodiments, the methods further comprise genetically editing the NK cells to reduce or eliminate expression of at least one endogenous gene or protein expressed as compared to a non-modified NK cell. In several embodiments, the genetic modification is performed prior to the first co-culturing. In several embodiments, the genetic modification comprises a disruption of a gene encoding CISH, thereby resulting in reduced or eliminated CIS expression by the NK cell.

In several embodiments, the methods further comprise engineering the NK express a chimeric antigen receptor that is directed against a tumor target and promotes cytotoxic activity against a tumor cell expressing the tumor target. In several embodiments the tumor target is selected from a ligand for the NKG2D receptor, CD19, CD70, CD38 or BCMA.

Also provided for herein is a use of the NK cells selected from a donor according to the methods disclosed herein or expanded by the methods disclosed herein for the preparation of a medicament for the treatment of cancer. Also provided for herein is a use of the NK cells selected from a donor according to the methods disclosed herein or expanded by the methods disclosed herein for the treatment of cancer.

Additionally provided for herein is a population of expanded immune cells for use in immunotherapy, comprising a population immune cells that were expanded in culture, wherein the immune cells express a chimeric antigen receptor that is directed against a tumor target, and wherein the immune cells are optionally genetically edited to reduce or eliminate expression of at least one gene endogenous to the immune cell, wherein the population of immune cells were expanded by a process comprising, consisting of, or consisting essentially of co-culturing, for a first time, in a culture media, a population of immune cells with a first batch of a feeder cell population, wherein the feeder cell population comprises cells engineered to express 4-1BBL and membrane-bound interleukin-15 (mbIL15), wherein the culture media is supplemented with at least soluble interleukin 12 (IL12) and soluble interleukin 18 (IL18), co-culturing, in the culture media, immune cells from the first co-culturing with a second batch of the feeder cell population, co-culturing, in the culture media, immune cells from the second co-culturing with a third batch of the feeder cell population, co-culturing, in the culture media, immune cells from the third co-culturing with a fourth batch of the feeder cell population, co-culturing, for a fifth time, in the culture media, immune cells from the fourth co-culturing with a fifth batch of the feeder cell population, wherein the culture media is supplemented with at least soluble IL12 and soluble IL18, and wherein a population of expanded immune cells results from the plurality of co-culturings.

In several embodiments, there is provided a population of expanded immune cells for use in immunotherapy, comprising a population immune cells that were expanded in culture, wherein the immune cells express aKIR and iKIR receptors and wherein the ratio of aKIR to iKIR expression prior to expansion was at least about 3, wherein the immune cells have been engineered to express a chimeric antigen receptor that is directed against a tumor target, and wherein the immune cells are optionally genetically edited to reduce or eliminate expression of at least one gene endogenous to the immune cell, wherein the population of immune cells were expanded by a process comprising, consisting of, or consisting essentially of co-culturing, for a first time, in a culture media, a population of immune cells with a first batch of a feeder cell population, wherein the feeder cell population comprises cells engineered to express 4-1BBL and membrane-bound interleukin-15 (mbIL15), wherein the culture media is supplemented with at least soluble interleukin 12 (IL12) and soluble interleukin 18 (IL18), co-culturing, a plurality of times, in the culture media, immune cells from a prior co-culturing with an additional batch of the feeder cell population, to generate a further expanded immune cell population, co-culturing, for a final time, in the culture media, at least a portion of the further expanded immune cells with an additional batch of the feeder cell population, wherein the culture media is supplemented with at least soluble IL12 and soluble IL18, and wherein a population of expanded immune cells results from the co-culturings.

In several embodiments, the population of expanded immune cells comprise NK cells. In several embodiments, the immune cells are edited to reduce or eliminate expression of CISH. In several embodiments, the CAR targets a ligand of the NKG2D receptor, CD19, CD38, BCMA or CD70.

Also provided for herein are methods for treating a cancer, comprising administering to a subject in need thereof a therapeutically effective amount of the population of expanded immune cells as provided for herein. Further provided are uses of a population of expanded immune cells as provided for herein for the preparation of a medicament for the treatment of cancer. Further provided are uses of a population of expanded immune cells as provided for herein for the treatment of cancer.

Additionally provided for herein is a method for treating cancer comprising administering to a subject a population NK cells that were expanded in culture, wherein the NK cells express a chimeric antigen receptor that is directed against a tumor target, and wherein the NK cells express reduced amounts of CISH as compared to a native NK cell, wherein the population of NK cells were expanded by a process comprising, consisting of, or consisting essentially of co-culturing, for a first time, in a culture media, a population of NK cells with a first batch of a feeder cell population, wherein the feeder cell population comprises cells engineered to express 4-1BBL and membrane-bound interleukin-15 (mbIL15), wherein the culture media is supplemented with at least soluble interleukin 12 (IL12) and soluble interleukin 18 (IL18), co-culturing, a plurality of times, in the culture media, NK cells from a prior co-culturing with an additional batch of the feeder cell population, to generate a further expanded NK cell population, co-culturing, for a final time, in the culture media, at least a portion of the further expanded NK cells with an additional batch of the feeder cell population, wherein the culture media is supplemented with at least soluble IL12 and soluble IL18.

### BRIEF DESCRIPTION OF THE DRAWINGS

The descriptions of the figures below are related to experiments and results that represent non-limiting embodiments of the inventions disclosed herein.

FIGS. 1A-1C relate to KIR expression on donor cells (1A, modified from Ewen et al Eur. J. Immunol. 2018. 48:355-365) and the effects of the presence or absence of stimulatory interleukins on expansion (1B) and cytotoxicity (1C) of NK cells from various donors, when untransduced, or when engineered to express an anti-CD19 chimeric antigen receptor (CAR).

FIGS. 2A-2F depict data related to trends of the cytotoxic potency and characterization of donor NK cells in view of the donor KIR haplotype and without culture supplementation with stimulatory interleukins (2A) or in the presence of stimulatory interleukins (2B).

FIG. 2C shows a summary of DNA-based high-resolution genotypic analysis of HLA & KIR performed on the 12 NK donors used to generate the data in 2A and 2B as well as KIR B content group determined using the IPD-KIR database. FIG. 2D shows data related to marker expression on NK cells expanded using engineered feeder cells with or without soluble IL12/IL18 cytokines and genetically modified with a retroviral CD19-CAR-mbIL-15 construct. Cells were characterized by flow cytometry on Day 0 & 14. FIG. 2E shows a volcano plot with changes of various markers in NK cells when expanded in the presence or the absence of IL12 and IL18. FIG. 2F shows gene expression data (with or without IL12 and IL18) and an upregulation of genes associated with activation of NK cells. FIGS. 2G-2J show cytotoxicity data for NK cells based on their KIR haplotype on different tumor cell lines.

FIGS. 3A-3B depict data related to correlation of donor NK cell potency and KIR haplotype and the impact of CMV status of the donor.

FIGS. 4A-4B show data related to the correlation of culture supplementation with stimulating interleukins and activating KIR haplotype.

FIGS. 5A-5B show data related to the correlation of culture supplementation with stimulating interleukins and inhibtory KIR haplotype.

FIGS. 6A-6B show data related to the correlation of donor cell expansion with cytotoxicity.

FIGS. 7A-7B show data related to the enhancement of cytotoxicity at an E:T of 1:4 based on culture media supplementation with stimulating interleukins.

FIGS. 8A-8B show additional data related to the discrimination between cytotoxicity of selected donor NK cells at an E:T of 1:8.

FIGS. 9A-9F show cytotoxicity data of expanded NK cells at day 21 of growth.

FIGS. 9A-9B show cytotoxicity curves for NK cells against tumor cells with and without stimulating interleukins. FIGS. 9C-9F show data related to the expression of various analytes in the supernatants from NALM-6 tumor cytotoxicity assays. FIG. 9C shows levels of IFN-g, FIG. 9D shows levels of GM-CSF, FIG. 9E shows levels of MIP-1a, and FIG. 9F shows levels of Perforin.

FIG. 10 shows data related to cytotoxicity quantification over 144 hours.

FIG. 11 shows data related to cytotoxicity quantification over 24 hours.

FIGS. 12A-12B show data related to the use of varied effector:target (E:T) ratios in order to discriminate among higher cytotoxicity donor cells.

FIG. 13 shows a schematic of an expansion protocol according to embodiments disclosed herein.

FIGS. 14A-14D show data related to expansion of cells using an embodiment of the expansion processes disclosed herein. 14A shows data from a first replicate of the experiment and 14B shows data from a replicate of the expansion from the day 56 time point (prior time points are same data as in 14A). FIGS. 14C and 14D tabulate the data of FIGS. 14A and 14B, respectively.

FIGS. 15A-15D show data related to fold expansion of cells using an embodiment of the expansion processes disclosed herein. 15A shows data from a first replicate of the experiment and 15B shows data from a replicate of the expansion from the fifth pulse (prior time points are same data as in 15A). FIGS. 15C and 15D tabulate the data of FIGS. 15A and 15B, respectively.

FIGS. 16A-16C show data summarizing the expansion of cells according to embodiments disclosed herein during the final 14 days of an expansion. FIG. 16A shows a first replicate of the expansion experiment, and FIG. 16B shows an additional replicate with the presence (solid) or absence (open) of IL12/18 at the inception of this final culture period. FIG. 16C tabulates the data of FIG. 16B.

FIGS. 17A-17B relate to CAR expression data. FIG. 17A tabulates data related to the percent of cells expressing the non-limiting CAR transduced into the cells earlier in the expansion process. FIG. 17B shows similar data for the percentage of cells expressing the CD19 CAR and mbIL15 at various stages of the expansion process as disclosed herein versus a pre-existing expansion approach (SP-standard process, also referred to as NKSTIM). MCB-Master Cell Bank; WCB-Working Cell Bank; FP-Final Product.

FIGS. 18A-18D summarize cytotoxicity of cell from various donors at the indicated point in the expansion process.

FIGS. 19A-19D show data related to the trends in expression of various markers during the expansion process. FIGS. 19A-19D show the trends of the expression of the listed markers with pulse number in NK cells from three donors. FIG. 19D shows expression of NKG2D (a non-limiting example of an activating receptor) in NK cells from three donors at various stages of the production methods disclosed herein, versus pre-expansion and Standard Process.

FIGS. 20A-20H show data related to expression of various markers by expanded cells. FIG. 20A-20D show data related to expression of the indicated markers on NK cells expanded according to embodiments disclosed herein with, or without, Il12 and IL18. FIGS. 20E-20F show data related to the expression of eomesodermin (Eomes) by NK cells expanded according to embodiments disclosed herein with, or without, IL12 and IL18. FIG. 20G shows expression of various markers of NK cell exhaustion by NK cells from a donor. FIG. 20H summarizes expressing of TIGIT across three donors at the WCB phase of expansion.

FIGS. 21A-21D show data related to expression of p16 by cells during the indicated points of an expansion.

FIGS. 22A-22F show data related to chromosomal stability and cytotoxicity of NK cells expanded according to methods disclosed herein. FIGS. 22A-22B show results of a chromosomal analysis of pre- and post-expansion NK cells. No chromosomal aberrations were observed after expansion. FIGS. 22C-22F show data related to the maintained cytotoxicity against tumor cell lines or non-tumor cell lines expressing CD19 by NK cells expanded according to embodiments disclosed herein.

FIGS. 23A-23E show data related to the expansion of NK cells from cord blood or peripheral blood using expansion methods as provided for herein. FIG. 23A shows data related to expansion of untransduced NK cells (either from cord or peripheral blood) over 14 days. FIG. 23B shows data related to the expansion of NK cells (either from cord or peripheral blood) engineered to express an anti-CD19 CAR over 14 days. FIG. 23C shows data related to expansion of NK cells either from cord or peripheral blood) engineered to express an anti-CD19 CAR over 70 days. FIG. 23D provides a summary of the fold expansion of NK cells for each phase of the expansion methods provided for herein. FIG. 23E shows data related to the degree of expansion with each reintroduction (e.g., “pulse”) of feeder cells.

FIGS. 24A-24M show data related to expression of various markers by the NK cells (either from cord or peripheral blood) during expansion. FIG. 24A shows expression of NKG2C. FIG. 24B shows expression of CD39. FIG. 24C shows expression of TIM3. FIG. 24D shows expression of OX40L. FIG. 24E shows expression of CD62L. FIG. 24F shows expression of LAG3. FIG. 24G shows expression of PD1. FIG. 24H shows expression of CD56. FIG. 24I shows expression of CD16. FIG. 24J shows expression of NKG2A. FIG. 24K shows expression of ILT2. FIG. 24L shows expression of CD57. FIG. 24M shows expression of TIGIT.

FIGS. 25A-25M show data related to expression of various additional markers by the NK cells (either from cord or peripheral blood) during expansion. FIG. 25A shows expression of KIR2DL2/L3. FIG. 25B shows expression of KIR2DS4. FIG. 25C shows expression of KIR2DL1/DS5. FIG. 25D shows expression of KIR3DS1. FIG. 25E shows expression of KIR2DL2/L3/S2. FIG. 25F shows expression of LAIR1. FIG. 25G shows expression of CD27. FIG. 25H shows expression of CD56. FIG. 25I shows expression of CD16. FIG. 25J shows expression of NKG2A. FIG. 25K shows expression of KIR3DL1. FIG. 25L shows expression of KLRG1. FIG. 25M shows expression of CD160.

FIGS. 26A-26M show data related to expression of various additional markers by the NK cells (either from cord or peripheral blood) during expansion. FIG. 26A shows expression of NKp30. FIG. 26B shows expression of 41BB. FIG. 26C shows expression of NKp80. FIG. 26D shows expression of NKp44. FIG. 26E shows expression of CD25. FIG. 26F shows expression of NKp46. FIG. 26G shows expression of DNAM1. FIG. 26H shows expression of CD56. FIG. 26I shows expression of CD16. FIG. 26J shows expression of 2B4. FIG. 26K shows expression of GITR. FIG. 26L shows expression of NKG2D. FIG. 26M shows expression of CD69.

FIGS. 27A-27B show data related to expression of various markers by NK cells during the expansion process. FIG. 27A shows the expression of CD57 and the NKG2C receptor during expansion of peripheral blood (PB) NK cells or cord blood (CB) NK cells. FIG. 27B shows the expression of KIRs and the NKG2A receptor during expansion of PB NK cells or CB NK cells.

FIGS. 28A-281 show data related to CD19 CAR expression on the PB NK or CB NK cells at 14 versus 70 days of expansion. FIG. 28A shows CD19 CAR expression on CB NK cells from a first donor expanded for 14 days. FIG. 28B shows CD19 CAR expression on CB NK cells from a second donor expanded for 14 days. FIG. 28C shows CD19 CAR expression on CB NK cells from a third donor expanded for 14 days. FIG. 28D shows CD19 CAR expression on CB NK cells from a fourth donor expanded for 14 days. FIG. 28E shows CD19 CAR expression on PB NK cells from a first donor expanded for 14 days. FIG. 28F shows CD19 CAR expression on CB NK cells from the third CB donor expanded for 70 days. FIG. 28G shows CD19 CAR expression on CB NK cells from the fourth CB donor expanded for 70 days. FIG. 28H shows CD19 CAR expression on PB NK cells from the first CB donor expanded for 70 days. FIG. 28I provides summary data of CD19 CAR expression for each donor at 14 or 70 days of expansion.

FIGS. 29A-29C shows data related to cytotoxicity for expanded NK cells. FIG. 29A shows cytotoxicity data of CB or PB NK cells expressing a CD19-directed CAR against Raji cells (Burkitt lymphoma, after 71 hours of co-culture) at the indicated E:T ratios. FIG. 29B shows cytotoxicity data of CB or PB NK cells expressing a CD19-directed CAR against NALM6 cells (B cell precursor leukemia, after 71 hours of co-culture) at the indicated E:T ratios. FIG. 29C shows cytotoxicity data of CB or PB NK cells expressing a CD19-directed CAR against HT-29-CD19 cells (colorectal adenocarcinoma engineered to ectopically express CD19, after 47 hours of co-culture) at the indicated E:T ratios.

FIGS. 30A-30C show data related to the cytotoxicity of NK cells (either CB NK or PB NK cells) after either 14 or 70 days of expansion according to methods provided for herein. FIG. 30A shows cytotoxicity data of CB or PB NK cells expanded for 14 or 70 days and expressing a CD19-directed CAR against Raji cells (Burkitt lymphoma, after 72 hours of co-culture) at the indicated E:T ratios. FIG. 30B shows cytotoxicity data of CB or PB NK cells expanded for 14 or 70 days and expressing a CD19-directed CAR against NALM6 cells (B cell precursor leukemia, after 72 hours of co-culture) at the indicated E:T ratios. FIG. 30C shows cytotoxicity data of CB or PB NK cells expanded for 14 or 70 days and expressing a CD19-directed CAR against HT-29-CD19 cells (colorectal adenocarcinoma engineered to ectopically express CD19, after 48 hours of co-culture) at the indicated E:T ratios.

### DETAILED DESCRIPTION

While cancer immunotherapy, or cellular therapy for other diseases, has advanced greatly in terms of the ability to engineer cells to express constructs of interest, there is still a need for clinically relevant number of those cells for patient administration. This is particularly important when the underlying native immune cell to be engineered and later administered is less prevalent than other immune cell types. This requires either starting with a larger amount of starting material, which may not be practical, or developing more efficient methods and compositions to expand (in some cases preferentially) the immune cell of interest, such as an NK cell. There are therefore provided herein, in several embodiments, methods for screening donors of immune cells for those who may exhibit a particular predisposition to enhanced expansion and/or enhanced cytotoxicity and compositions and methods that advantageously allow for the unexpectedly robust expansion of NK cells (or other immune cells).

In several embodiments, there are provided populations of expanded and activated NK cells derived from co-culturing a modified “feeder” cell disclosed herein with a starting population of immune cells and supplementing the co-culture with various cytokines at certain time points during the expansion.

### Donor Selection-Characteristics and Methods of Selecting Donor

As many types of cancer immunotherapy rely of donor-derived cells, in particular in the allogeneic context, the selection of a donor can be a key component of generation of a successful therapeutic regimen. As discussed in more detail herein, in several embodiments, allogeneic donors are used, for example in the development of off the shelf cancer immunotherapies, in particular those one or more types of immune cell, such as Natural Killer (NK) and/or T cells.

Various donor characteristics can be evaluated, alone or in combination, in order to improve one or more aspects of the donor-derived cells. For example, in several embodiments, an optimal donor would exhibit one or more of (i) predisposed to expansion in culture, (ii) readily transduced (e.g., with a vector for delivery of a chimeric antigen receptor (CAR) or other payload (gene editing machinery), and (iii) potent baseline cytotoxicity. One (or combinations) of these, or other, characteristics discussed herein may be a weighted factor in making a given donor an optimal candidate from which to develop a master cell bank (MCB) and/or a working cell bank (WCB) such that a single donor can yield numerous identical doses of cells for use in allogeneic cell therapy.

As will be discussed in more detail below, and in the examples, multiple approaches can be used to evaluate and screen potential donors. For example, in several embodiments, protein expression techniques, such as flow cytometry to measure certain cell surface markers is used. In several embodiments, various assays are used to measure the cytokine secretome of a cell, or determine its chemokine/granule release potential. In several embodiments, gene expression is evaluated to determine what potential genes that could impact or hinder cell expansion are expressed. In several embodiments, cells from a potential donor are genotyped, for example with respect to their HLA profile or Killer Cell Ig-like Receptors (KIR) profile. In several embodiments, the memory-like characteristics (e.g., memory or memory-like NK cell characteristics) are evaluated (e.g., cytomegalovirus positivity of donor, NKG2C expression, and/or ability for clonal expansion). In several embodiments, combinations of such methods are used. In several embodiments, such methods can be used for correlating one or more of the characteristics assessed with potency and/or ability for expansion.

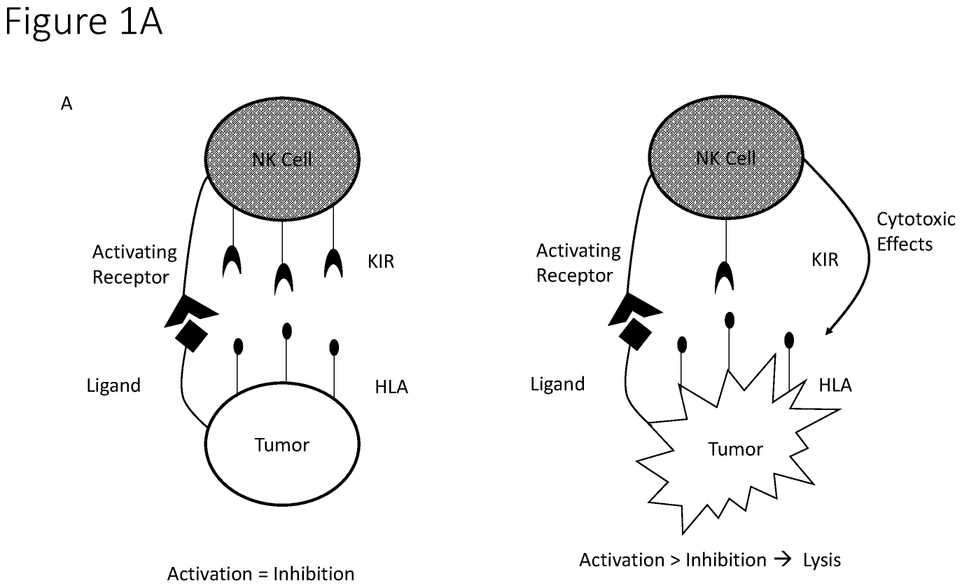

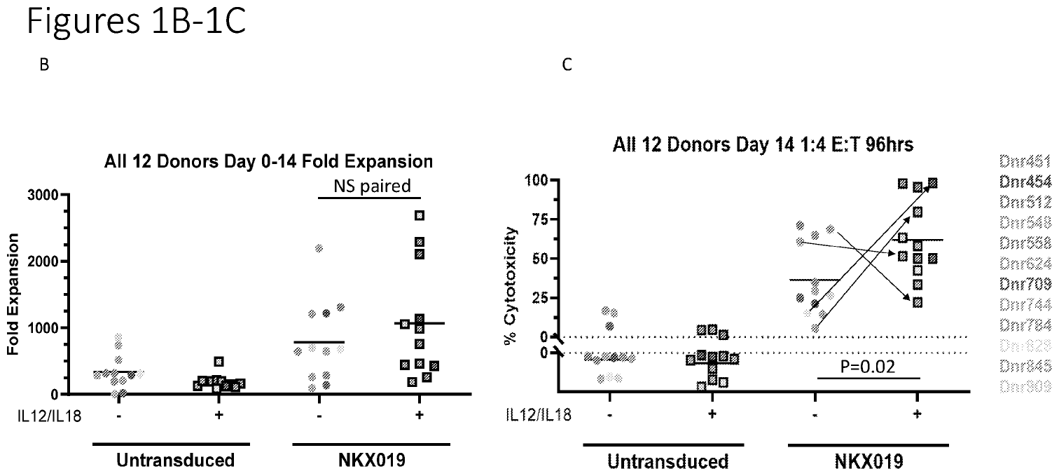

In several embodiments, as disclosed herein, NK cells are collected from a donor, engineered and/or edited and expanded in culture for use in cellular therapy. NK cell functions are regulated by a diversity of activating and inhibitory cell surface receptors. As mentioned above, one of these cell surface receptor families controlling the effector function of NK cells are the KIRs. Six of them are activating KIRs (aKIR), including KIR2DS1, KIR2DS2, KIR2DS3, KIR2DS4, KIR2DS15, and KIR3DS1. In contrast, seven are inhibitory KIRs (iKIR), including KIR2DL1, KIR2DL2, KIR2DL3, KIR3DL1, KIR3DL2, KIR3DL3, and KIR2DL5). KIR2DL4 exhibits both activating and inhibitory properties. Finally, two are believed to be pseudogenes (KIR2DP1 and KIR3DP1). In mature NK cells, iKIR inhibit cytotoxicity if bound to HLA (and other) tumor ligands while aKIR increase cytotoxicity if bound to HLA (and other) tumor ligands (see FIG. 1A, modified from Ewen et al Eur. J. Immunol. 2018. 48:355-365). KIRs may either inhibit or stimulate NK cell activity after engagement with specific human leukocyte antigen (HLA) class I ligands and, despite their high genetic variability and particularly diverse KIR/HLA ligand interactions, the KIRs allow the NK cells to self-discriminate healthy cells from transformed or pathogen-infected cells and regulate their effector function. Thus, according to several embodiments, the NK cells (or other immune cells collected from a potential donor) are evaluated with respect to their KIR profile, including in one embodiment assessing aKIR expression, in one embodiment assessing iKIR expression, and in several embodiments, assessing both aKIR and iKIR expression and calculating a ratio that is predictive of the future expandability and/or cytotoxicity of the cells.

As discussed in more detail below in the examples, according to several embodiments, donor potency (e.g., eventual cytotoxicity) can be driven by KIR-based or non-KIR-based factors. KIR drives potency via two different mechanisms, according to some embodiments. In several embodiments, there is a mismatch of donor (and thus therapeutic) cell iKIR expression with a patient tumor HLA. This mismatch means that the patient's tumor cells do not engage the iKIR (and thus less or no NK cell inhibition results) and therefore the tumor cells are more readily killed. Alternatively, or in addition to, the above, those donors who are KIR Haplotype Group B exhibit higher frequencies of activating KIR are thus more potent, according to several embodiments. Non-KIR-based potency, according to some embodiments, exhibit a robust response to stimulatory molecules (such as IL12 and/or IL18) that are used in certain embodiments of immune cell expansion, which imparts to them enhanced cytotoxicity. In some embodiments, a donor is preferred because their cells exhibit both KIR and non-KIR-based potency increases (e.g., after expansion).

In several embodiments, a candidate donor is identified and a blood sample comprising immune cells is obtained from the candidate donor. In several embodiments, the sample is divided into multiple portions, with one or more being subjected to a screening process, and the others being saved and subsequently used as donor cells for expansion or discarded. In several embodiments, the immune cells are separated to at least in part, substantially or completely isolated NK cells. In several embodiments, the expression of at least one of KIR2DS1, KIR2DS2, KIR2DS3, KIR2DS4, KIR2DS15, and KIR3DS1 is evaluated. In several embodiments, the expression of at least one of KIR2DL1, KIR2DL2, KIR2DL3, KIR3DL1, KIR3DL2, KIR3DL3, and KIR2DL5 is evaluated. In several embodiments, the expression of at least one of KIR2DS1, KIR2DS2, KIR2DS3, KIR2DS4, KIR2DS15, and KIR3DS1 is evaluated and also the expression of at least one of KIR2DL1, KIR2DL2, KIR2DL3, KIR3DL1, KIR3DL2, KIR3DL3, and KIR2DL5 is evaluated. In several embodiments, wherein expression of both at least one aKIR and at least one iKIR is evaluated, a comparison of the amount of aKIR to the amount of iKIR is made. In several embodiments, a raw expression signal comparison is used (e.g., signal intensities). In several embodiments, normalizations of expression are performed, e.g., to a housekeeping gene/protein. In several embodiments, a ratio of aKIR to iKIR expression is calculated. In several embodiments, the ratio is predictive of the future potency of the cells, as it represents the probability that an NK cell will generate greater activating KIR function versus inhibitory KIR function. In several embodiments, a candidate donor with an aKIR:iKIR ratio of at least about 3:1, about 3.5:1, about 4:1, about 4.5:1, about 5:1, about 5.5:1, about 6:1, about 6.5:1, about 7:1, about 7.5:1, about 8:1, about 8.5:1 or greater (and including any ratio between those listed) is determined to be a preferred donor (a donor whose cells are later engineered/edited and/or expanded). In several embodiments, a preferred donor has an aKIR:iKIR ratio of about 3:1, 5:1, 8:1, 10:1, 12:1, 15:1, 18:1, 20:1 or greater (including any ratio between those listed). In several embodiments, a donor can be selected based on the number of aKIRs that are expressed. In several embodiments, a candidate donor can be determined to be a preferred donor based on the donor's cells expressing at least 2, at least 3, or at least 4 aKIRs. In several embodiments, a preferred donor population of cells will express fewer than a full contingent of iKIRs, for example less than 5, less than 4, less than 3 or less than 2 iKIRs.

### Cells for Use in Immune Cell Expansion

Some embodiments of the methods and compositions provided herein relate to collection of a cell such as an immune cell, for example from a donor, and expansion of all or a subset of the collected cells in culture. In addition, in several embodiments, the cells are engineered and/or gene edit for use in, for example, cancer immunotherapy. For example, an immune cell, such as a T cell, may be engineered to include a chimeric receptor such as a CD19-directed chimeric receptor, or engineered to include a nucleic acid encoding said chimeric receptor as described herein. Additional embodiments relate to engineering a second set of cells to express another cytotoxic receptor complex, such as an NKG2D chimeric receptor complex as disclosed herein. Still additional embodiments relate to the further genetic manipulation of T cells (e.g., donor T cells) to reduce, disrupt, minimize and/or eliminate the ability of the donor T cell to be alloreactive against recipient cells (graft versus host disease).

Traditional anti-cancer therapies relied on a surgical approach, radiation therapy, chemotherapy, or combinations of these methods. As research led to a greater understanding of some of the mechanisms of certain cancers, this knowledge was leveraged to develop targeted cancer therapies. Targeted therapy is a cancer treatment that employs certain drugs that target specific genes or proteins found in cancer cells or cells supporting cancer growth, (like blood vessel cells) to reduce or arrest cancer cell growth. More recently, genetic engineering has enabled approaches to be developed that harness certain aspects of the immune system to fight cancers. In some cases, a patient's own immune cells are modified to specifically eradicate that patient's type of cancer. Various types of immune cells can be used, such as T cells, Natural Killer (NK cells), or combinations thereof, as described in more detail below.

To facilitate cancer immunotherapies, there are provided for herein polynucleotides, polypeptides, and vectors that encode chimeric antigen receptors (CAR) that comprise a target binding moiety (e.g., an extracellular binder of a ligand, or a tumor marker-directed chimeric receptor, expressed by a cancer cell) and a cytotoxic signaling complex. For example, some embodiments include a polynucleotide, polypeptide, or vector that encodes, for example a chimeric antigen receptor directed against a tumor marker, for example, CD19, CD38, CD123, CD70, Her2, mesothelin, Claudin 6, BCMA, EGFR, among others, to facilitate targeting of an immune cell to a cancer and exerting cytotoxic effects on the cancer cell. Also provided are engineered immune cells (e.g., T cells or NK cells) expressing such CARs. There are also provided herein, in several embodiments, polynucleotides, polypeptides, and vectors that encode a construct comprising an extracellular domain comprising two or more subdomains, e.g., first CD19-targeting subdomain comprising a CD19 binding moiety as disclosed herein and a second subdomain comprising a C-type lectin-like receptor and a cytotoxic signaling complex. Also provided are engineered immune cells (e.g., T cells or NK cells) expressing such bi-specific constructs. Methods of treating cancer and other uses of such cells for cancer immunotherapy are also provided for herein.

Non-limiting examples of CAR constructs for expression in cells provided for herein are provided in Table 1 below:

| | SEQ |
|---|---|
| Identifier | ID NO |
| NKG2D Chimeric Receptor | 1 |
| with mbIL15 DNA | |
| NKG2D Chimeric Receptor | 2 |
| Amino Acid with mbIL15 | |
| DNA NK19H-No Flag-1 with | 3 |
| mbIL15 | |
| AA NK19H-No Flag-1 with | 4 |
| mbIL15 | |
| DNA NK19H-No Flag-2 with | 5 |
| mbIL 15 | |
| AA NK19H-No Flag-2 with | 6 |
| mbIL 15 | |
| DNA NK19H-No Flag-3 with | 7 |
| mbIL15 | |
| AA NK19H-No Flag-3 with | 8 |
| mbIL15 | |
| DNA NK19H-No Flag-4 with | 9 |
| mbIL15 | |
| AA NK19H-No Flag-4 with | 10 |
| mbIL15 | |
| DNA NK19H-No Flag-5 with | 11 |
| mbIL15 | |
| AA NK19H-No Flag-5 with | 12 |
| mbIL15 | |
| DNA NK19H-No Flag-6 with | 13 |
| mbIL15 | |
| AA NK19H-No Flag-6 with | 14 |
| mbIL15 | |
| DNA NK19H-No Flag-7 with | 15 |
| mbIL15 | |
| AA NK19H-No Flag-7 with | 16 |
| mbIL15 | |
| DNA NK19H-No Flag-8 with | 17 |
| mbIL15 | |
| AA NK19H-No Flag-8 with | 18 |
| mbIL15 | |
| DNA NK19H-No Flag-9 with | 19 |
| mbIL15 | |
| AA NK19H-No Flag-9 with | 20 |
| mbIL15 | |
| DNA NK19H-No Flag-10 with | 21 |
| mbIL15 | |
| AA NK19H-No Flag-10 with | 22 |
| mbIL15 | |
| DNA NK19H-No Flag-11 with | 23 |
| mbIL15 | |
| AA NK19H-No Flag-11 with | 24 |
| mbIL15 | |
| DNA NK19H-No Flag-12 with | 25 |
| mbIL15 | |
| AA NK19H-No Flag-12 with | 26 |
| mbIL15 | |
| DNA NK19-1 | 27 |
| AA NK19-1 | 28 |
| NKG2D Chimeric Receptor | 40 |
| DNA | |
| NKG2D Chimeric Receptor | 41 |
| Amino Acid | |
| DNA | 42 |
| NK77.71_CD70_DB04_D02 | |
| with mbIL15 | |
| AA | 43 |
| NK77.71_CD70_DB04_D02 | |
| DNA | 44 |
| NK77.17_CD70_DB02_B06 | |
| with mbIL15 | |
| AA | 45 |
| NK77.17_CD70_DB02_B06 | |
| DNA | 46 |
| NK77.58_CD70_DB03_H08 | |
| with mbIL15 | |
| AA | 47 |
| NK77.58_CD70_DB03_H08 | |
| AA NK19H-No Flag-1 | 48 |
| AA NK19H-No Flag-2 | 49 |
| AA NK19H-No Flag-3 | 50 |
| AA NK19H-No Flag-4 | 51 |
| AA NK19H-No Flag-5 | 52 |
| AA NK19H-No Flag-6 | 53 |
| AA NK19H-No Flag-7 | 54 |
| AA NK19H-No Flag-8 | 55 |
| AA NK19H-No Flag-9 | 56 |
| AA NK19H-No Flag-10 | 57 |
| AA NK19H-No Flag-11 | 58 |
| AA NK19H-No Flag-12 | 59 |
| mbIL 15 DNA | 60 |
| mbIL 15 AA | 61 |
| NK77.17_CD70_DB02_B06 | 62 |
| construct [+mbIL 15] | |
| NK77.58_CD70_DB03_H08 | 63 |
| construct [+mbIL 15] | |
| NK77.71_CD70_DB04_D02 | 64 |
| construct [+mbIL 15] | |
| NK77.17_CD70_DB02_B06 | 65 |
| construct [no FLAG/GS2; | |
| +mbIL15] | |
| NK77.58_CD70_DB03_H08 | 66 |
| construct [no FLAG/GS2; | |
| +mbIL15] | |
| NK77.71_CD70_DB04_D02 | 67 |
| construct [no FLAG/GS2; | |
| +mbIL15] | |
| NK77.17_CD70_DB02_B06 | 68 |
| CAR only [no | |
| mbIL15/FLAG/GS2] | |
| NK77.58_CD70_DB03_H08 | 69 |
| CAR only [no | |
| mbIL15/FLAG/GS2] | |
| NK77.71_CD70_DB04_D02 | 70 |
| CAR only [no | |
| mbIL15/FLAG/GS2] | |

To facilitate cancer immunotherapies, there are also provided for herein polynucleotides, polypeptides, and vectors that encode chimeric receptors that comprise a target binding moiety (e.g., an extracellular binder of a ligand expressed by a cancer cell) and a cytotoxic signaling complex. For example, some embodiments include a polynucleotide, polypeptide, or vector that encodes, for example an activating chimeric receptor comprising an NKG2D extracellular domain that is directed against a tumor marker, for example, MICA, MICB, ULBP1, ULBP2, ULBP3, ULBP4, ULBP5, and ULBP6, among others, to facilitate targeting of an immune cell to a cancer and exerting cytotoxic effects on the cancer cell. Also provided are engineered immune cells (e.g., T cells or NK cells) expressing such chimeric receptors. There are also provided herein, in several embodiments, polynucleotides, polypeptides, and vectors that encode a construct comprising an extracellular domain comprising two or more subdomains, e.g., first and second ligand binding receptor and a cytotoxic signaling complex. Also provided are engineered immune cells (e.g., T cells or NK cells) expressing such bi-specific constructs (in some embodiments the first and second ligand binding domain target the same ligand). Methods of treating cancer and other uses of such cells for cancer immunotherapy are also provided for herein.

### Engineered Cells

In several embodiments, cells of the immune system are engineered to have enhanced cytotoxic effects against target cells, such as tumor cells. For example, a cell of the immune system may be engineered to include a tumor-directed chimeric receptor and/or a tumor-directed CAR as described herein. In several embodiments, white blood cells or leukocytes, are used, since their native function is to defend the body against growth of abnormal cells and infectious disease. There are a variety of types of white bloods cells that serve specific roles in the human immune system, and are therefore a preferred starting point for the engineering of cells disclosed herein. White blood cells include granulocytes and agranulocytes (presence or absence of granules in the cytoplasm, respectively). Granulocytes include basophils, eosinophils, neutrophils, and mast cells. Agranulocytes include lymphocytes and monocytes. Cells such as those that follow or are otherwise described herein may be engineered to include a chimeric receptor, such as an NKG2D chimeric receptor, and/or a CAR, such as a CD19-directed CAR, or a nucleic acid encoding the chimeric receptor or the CAR. In several embodiments, the cells are optionally engineered to co-express a membrane-bound interleukin 15 (mbIL15) co-stimulatory domain. As discussed in more detail below, in several embodiments, the cells, particularly T cells, are further genetically modified to reduce and/or eliminate the alloreactivity of the cells.

### Monocytes

Monocytes are a subtype of leukocyte. Monocytes can differentiate into macrophages and myeloid lineage dendritic cells. Monocytes are associated with the adaptive immune system and serve the main functions of phagocytosis, antigen presentation, and cytokine production. Phagocytosis is the process of uptake of cellular material, or entire cells, followed by digestion and destruction of the engulfed cellular material. In several embodiments, monocytes are used in connection with one or more additional engineered cells as disclosed herein. Some embodiments of the methods and compositions described herein relate to a monocyte that includes a tumor-directed CAR, or a nucleic acid encoding the tumor-directed CAR. Several embodiments of the methods and compositions disclosed herein relate to monocytes engineered to express a CAR that targets a tumor marker, for example, CD19, CD38, CD123, CD70, Her2, mesothelin, Claudin 6, BCMA, EGFR, among any of the others disclosed herein, and a membrane-bound interleukin 15 (mbIL15) co-stimulatory domain. Several embodiments of the methods and compositions disclosed herein relate to monocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### Lymphocytes

Lymphocytes, the other primary sub-type of leukocyte include T cells (cell-mediated, cytotoxic adaptive immunity), natural killer cells (cell-mediated, cytotoxic innate immunity), and B cells (humoral, antibody-driven adaptive immunity). While B cells are engineered according to several embodiments, disclosed herein, several embodiments also relate to engineered T cells or engineered NK cells (mixtures of T cells and NK cells are used in some embodiments, either from the same donor, or different donors). Several embodiments of the methods and compositions disclosed herein relate to lymphocytes engineered to express a CAR that targets a tumor marker, for example, CD19, CD38, CD123, CD70, Her2, mesothelin, Claudin 6, BCMA, EGFR, among any of the others disclosed herein, and a membrane-bound interleukin 15 (mbIL15) co-stimulatory domain. Several embodiments of the methods and compositions disclosed herein relate to lymphocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### T Cells

T cells are distinguishable from other lymphocytes sub-types (e.g., B cells or NK cells) based on the presence of a T-cell receptor on the cell surface. T cells can be divided into various different subtypes, including effector T cells, helper T cells, cytotoxic T cells, memory T cells, regulatory T cells, natural killer T cell, mucosal associated invariant T cells and gamma delta T cells. In some embodiments, a specific subtype of T cell is engineered. In some embodiments, a mixed pool of T cell subtypes is engineered. In some embodiments, there is no specific selection of a type of T cells to be engineered to express the cytotoxic receptor complexes disclosed herein. In several embodiments, specific techniques, such as use of cytokine stimulation are used to enhance expansion/collection of T cells with a specific marker profile. For example, in several embodiments, activation of certain human T cells, e.g. CD4+ T cells, CD8+ T cells is achieved through use of CD3 and/or CD28 as stimulatory molecules. In several embodiments, there is provided a method of treating or preventing cancer or an infectious disease, comprising administering a therapeutically effective amount of T cells expressing the cytotoxic receptor complex and/or a homing moiety as described herein. In several embodiments, the engineered T cells are autologous cells, while in some embodiments, the T cells are allogeneic cells. Several embodiments of the methods and compositions disclosed herein relate to T cells engineered to express a CAR that targets a tumor marker, for example, CD19, CD38, CD123, CD70, Her2, mesothelin, Claudin 6, BCMA, EGFR, among any of the others as disclosed herein, and a membrane-bound interleukin 15 (mbIL15) co-stimulatory domain. Several embodiments of the methods and compositions disclosed herein relate to T cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### NK Cells

In several embodiments, there is provided a method of treating or preventing cancer or an infectious disease, comprising administering a therapeutically effective amount of natural killer (NK) cells expressing the cytotoxic receptor complex and/or a homing moiety as described herein. In several embodiments, the engineered NK cells are autologous cells, while in some embodiments, the NK cells are allogeneic cells. In several embodiments, NK cells are preferred because the natural cytotoxic potential of NK cells is relatively high. In several embodiments, it is unexpectedly beneficial that the engineered cells disclosed herein can further upregulate the cytotoxic activity of NK cells, leading to an even more effective activity against target cells (e.g., tumor or other diseased cells). Some embodiments of the methods and compositions described herein relate to NK cells engineered to express a CAR that targets a tumor marker, for example, CD19, CD38, CD123, CD70, Her2, mesothelin, Claudin 6, BCMA, EGFR, among any of the others disclosed herein, and optionally a membrane-bound interleukin 15 (mbIL15) co-stimulatory domain. Several embodiments of the methods and compositions disclosed herein relate to NK cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain. In some embodiments, the NK cells are derived from cell line NK-92. NK-92 cells are derived from NK cells, but lack major inhibitory receptors displayed by normal NK cells, while retaining the majority of activating receptors. Some embodiments of NK-92 cells described herein related to NK-92 cell engineered to silence certain additional inhibitory receptors, for example, SMAD3, allowing for upregulation of interferon-γ (IFNγ), granzyme B, and/or perforin production. Additional information relating to the NK-92 cell line is disclosed in WO 1998/49268 and U.S. Patent Application Publication No. 2002-0068044 and incorporated in their entireties herein by reference. NK-92 cells are used, in several embodiments, in combination with one or more of the other cell types disclosed herein. For example, in one embodiment, NK-92 cells are used in combination with NK cells as disclosed herein. In an additional embodiment, NK-92 cells are used in combination with T cells as disclosed herein.

### Hematopoietic Stem Cells

In some embodiments, hematopoietic stem cells (HSCs) are used in the methods of immunotherapy disclosed herein. In several embodiments, the cells are engineered to express a homing moiety and/or a cytotoxic receptor complex. HSCs are used, in several embodiments, to leverage their ability to engraft for long-term blood cell production, which could result in a sustained source of targeted anti-cancer effector cells, for example to combat cancer remissions. In several embodiments, this ongoing production helps to offset anergy or exhaustion of other cell types, for example due to the tumor microenvironment. In several embodiments allogeneic HSCs are used, while in some embodiments, autologous HSCs are used. In several embodiments, HSCs are used in combination with one or more additional engineered cell type disclosed herein. Some embodiments of the methods and compositions described herein relate to a stem cell, such as a hematopoietic stem cell engineered to express a CAR that targets a tumor marker, for example, CD19, CD38, CD123, CD70, Her2, mesothelin, Claudin 6, BCMA, EGFR, among any of the others disclosed herein, and optionally a membrane-bound interleukin 15 (mbIL15) co-stimulatory domain. Several embodiments of the methods and compositions disclosed herein relate to hematopoietic stem cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### Induced Pluripotent Stem Cells

In some embodiments, induced pluripotent stem cells (iPSCs) are used in the method of immunotherapy disclosed herein. iPSCs are used, in several embodiments, to leverage their ability to differentiate and derive into non-pluripotent cells, including, but not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells comprising one or several genetic modifications at selected sites through differentiating iPSCs or less differentiated cells comprising the same genetic modifications at the same selected sites. In several embodiments, the iPSCs are used to generate iPSC-derived NK or T cells. In several embodiments, the cells are engineered to express a homing moiety and/or a cytotoxic receptor complex. In several embodiments, iPSCs are used in combination with one or more additional engineered cell type disclosed herein. Some embodiments of the methods and compositions described herein relate to a stem cell, such as a induced pluripotent stem cell engineered to express a CAR that targets a tumor marker, for example, CD19, CD38, CD123, CD70, Her2, mesothelin, Claudin 6, BCMA, EGFR, among any of the others disclosed herein, and optionally a membrane-bound interleukin 15 (mbIL15) co-stimulatory domain. Several embodiments of the methods and compositions disclosed herein relate to induced pluripotent stem cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### Genetic Editing of Cells

As discussed herein, a variety of cell types can be utilized in cellular immunotherapy. Several embodiments disclosed herein relate to the identification of donors whose cells are particularly disposed to efficient expansion in culture or exhibit particularly robust cytotoxicity against target tumor cells (e.g., when engineered to express a CAR). Further, genetic modifications can be made to these cells in order to enhance one or more aspects of their efficacy (e.g., cytotoxicity) and/or persistence (e.g., active life span). As discussed herein, in several embodiments NK cells are used for immunotherapy. In several embodiments provided for herein, gene editing of the NK cell can advantageously impart to the edited NK cell the ability to resist and/or overcome various inhibitory signals that are generated in the tumor microenvironment. It is known that tumors generate a variety of signaling molecules that are intended to reduce the anti-tumor effects of immune cells. As discussed in more detail below, in several embodiments, gene editing of the NK cell limits this tumor microenvironment suppressive effect on the NK cells, T cells, combinations of NK and T cells, or any edited/engineered immune cell provided for herein. As discussed below, in several embodiments, gene editing is employed to reduce or knockout expression of target proteins, for example by disrupting the underlying gene encoding the protein. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, the gene is completely knocked out, such that expression of the target protein is undetectable. In several embodiments, gene editing is used to “knock in” or otherwise enhance expression of a target protein. In several embodiments, expression of a target protein can be enhanced by about 30%, about 40%, about 50%, about 60%, about 70%, about 75%, about 80%, about 85%, about 90%, about 95%, about 97%, about 98%, about 99%, or more (including any amount between those listed). Unless indicated otherwise to the contrary, the sequences provided for guide RNAs that are recited using deoxyribonucleotides refer to the target DNA and shall be considered as also referencing those guides used in practice (e.g., employing ribonucleotides, where the ribonucleotide uracil is used in lieu of deoxyribonucleotide thymine or vice-versa where thymine is used in lieu of uracil, wherein both are complementary base pairs to adenine when reciting either an RNA or DNA sequence). For example, a gRNA with the sequence ATGCTCAATGCGTC shall also refer to the following sequence AUGCUCAAUGCGUC or a gRNA with sequence AUGCUCAAUGCGUC shall also refer to the following sequence ATGCTCAATGCGTC.

By way of non-limiting example, modulators of one or more aspects of NK cell (or T cell) function are modulated through gene editing. A variety of cytokines impart either negative (as with TGF-beta in more detail below) or positive signals to immune cells. By way of non-limiting example, IL15 is a positive regulator of NK cells, which as disclosed herein, can enhance one or more of NK cell homing, NK cell migration, NK cell expansion/proliferation, NK cell cytotoxicity, and/or NK cell persistence. To keep NK cells in check under normal physiological circumstances, a cytokine-inducible SH2-containing protein (CIS, encoded by the CISH gene) acts as a critical negative regulator of IL-15 signaling in NK cells. As discussed herein, because IL15 biology impacts multiple aspects of NK cell functionality, including, but not limited to, proliferation/expansion, activation, cytotoxicity, persistence, homing, migration, among others. Thus, according to several embodiments, editing CISH enhances the functionality of NK cells across multiple functionalities, leading to a more effective and long-lasting NK cell therapeutic. In several embodiments, inhibitors of CIS are used in conjunction with engineered NK cell administration. In several embodiments, the CIS expression is knocked down or knocked out through gene editing of the CISH gene, for example, by use of CRISPR-Cas editing. Small interfering RNA, antisense RNA, TALENs or zinc fingers are used in other embodiments. In some embodiments CIS expression in T cells is knocked down through gene editing. Non-limiting examples of guide RNAs that can target an endonuclease, such as Cas9, to edit a CISH gene are provided in Table 2, below (additional information on CISH editing can be found, for example in International Patent Application No. PCT/US2020/035752, which is incorporated in its entirety by reference herein).

TABLE 2
CISH Guide RNAs

| SEQ ID NO: | Name | Sequence | Target |
|---|---|---|---|
| 29 | CISH-1 | CTCACCAGATTCCCGAAGGT | Exon 2 |
| 30 | CISH-2 | CCGCCTTGTCATCAACCGTC | Exon 3 |
| 31 | CISH-3 | TCTGCGTTCAGGGGTAAGCG | Exon 1 |
| 32 | CISH-4 | GCGCTTACCCCTGAACGCAG | Exon 1 |
| 33 | CISH-5 | CGCAGAGGACCATGTCCCCG | Exon 1 |

In several embodiments, CISH gene editing endows an NK cell with enhanced ability to home to a target site. In several embodiments, CISH gene editing endows an NK cell with enhanced ability to migrate, e.g., within a tissue in response to, for example chemoattractants or away from repellants. In several embodiments, CISH gene editing endows an NK cell with enhanced ability to be activated, and thus exert, for example, anti-tumor effects. In several embodiments, CISH gene editing endows an NK cell with enhanced proliferative ability, which in several embodiments, allows for generation of robust NK cell numbers from a donor blood sample. In addition, in such embodiments, NK cells edited for CISH and engineered to express a CAR are more readily, robustly, and consistently expanded in culture. In several embodiments, CISH gene editing endows an NK cell with enhanced cytotoxicity. In several embodiments, the editing of CISH synergistically enhances the cytotoxic effects of engineered NK cells and/or engineered T cells that express a CAR.

In several embodiments, CISH gene editing activates or inhibits a wide variety of pathways. The CIS protein is a negative regulator of IL15 signaling by way of, for example, inhibiting JAK-STAT signaling pathways. These pathways would typically lead to transcription of IL15-responsive genes (including CISH). In several embodiments, knockdown of CISH disinhibits JAK-STAT (e.g., JAK1-STAT5) signaling and there is enhanced transcription of IL15-responsive genes. In several embodiments, knockout of CISH yields enhanced signaling through mammalian target of rapamycin (mTOR), with corresponding increases in expression of genes related to cell metabolism and respiration. In several embodiments, knockout of CISH yields IL15 induced increased expression of IL-2Ra (CD25), but not IL-15Ra or IL-2/15RB, enhanced NK cell membrane binding of IL15 and/or IL2, increased phosphorylation of STAT-3 and/or STAT-5, and elevated expression of the antiapoptotic proteins, such as Bcl-2. In several embodiments, CISH knockout results in IL15-induced upregulation of selected genes related to mitochondrial functions (e.g., electron transport chain and cellular respiration) and cell cycle. Thus, in several embodiments, knockout of CISH by gene editing enhances the NK cell cytotoxicity and/or persistence, at least in part via metabolic reprogramming. In several embodiments, negative regulators of cellular metabolism, such as TXNIP, are downregulated in response to CISH knockout. In several embodiments, promotors for cell survival and proliferation including BIRC5 (Survivin), TOP2A, CKS2, and RACGAP1 are upregulated after CISH knockout, whereas antiproliferative or proapoptotic proteins such as TGFB1, ATM, and PTCH1 are downregulated. In several embodiments, CISH knockout alters the state (e.g., activates or inactivates) signaling via or through one or more of CXCL-10, IL2, TNF, IFNg, IL13, IL4, Jnk, PRF1, STAT5, PRKCQ, IL2 receptor Beta, SOCS2, MYD88, STAT3, STAT1, TBX21, LCK, JAK3, IL& receptor, ABL1, IL9, STAT5A, STAT5B, Tcf7, PRDM1, and/or EOMES.

In several embodiments, gene editing of the immune cells can also provide unexpected enhancement in the expansion, persistence and/or cytotoxicity of the edited immune cell. As disclosed herein, engineered cells (e.g., those expressing a CAR) may also be edited, the combination of which provides for a robust cell for immunotherapy. In several embodiments, the edits allow for unexpectedly improved NK cell expansion, persistence and/or cytotoxicity. In several embodiments, knockout of CISH expression in NK cells removes a potent negative regulator of IL15-mediated signaling in NK cells, disinhibits the NK cells and allows for one or more of enhanced NK cell homing, NK cell migration, activation of NK cells, expansion, cytotoxicity and/or persistence. Additionally, in several embodiments, the editing can enhance NK and/or T cell function in the otherwise suppressive tumor microenvironment. In several embodiments, CISH gene editing results in enhanced NK cell expansion, persistence and/or cytotoxicity without requiring Notch ligand being provided exogenously.

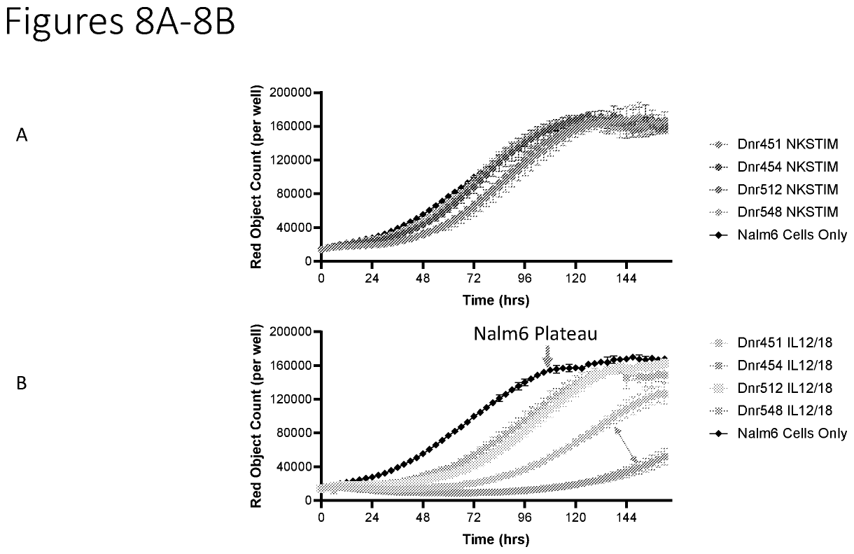

As mentioned above, TGF-beta is one a cytokine released by tumor cells that results in immune suppression within the tumor microenvironment. That immune suppression reduces the ability of immune cells, even engineered CAR-immune cells is some cases, to destroy the tumor cells, thus allowing for tumor progression. In several embodiments, as discussed in detail below, immune checkpoint inhibitors are disrupted through gene editing. In several embodiments, blockers of immune suppressing cytokines in the tumor microenvironment are used, including blockers of their release or competitive inhibitors that reduce the ability of the signaling molecule to bind and inhibit an immune cell. Such signaling molecules include, but are not limited to TGF-beta, IL10, arginase, inducible NOS, reactive-NOS, Arg1, Indoleamine 2,3-dioxygenase (IDO), and PGE2. However, in additional embodiments, there are provided immune cells, such as NK cells, wherein the ability of the NK cell (or other cell) to respond to a given immunosuppressive signaling molecule is disrupted and/or eliminated. For example, in several embodiments, in several embodiments, NK cells or T cells are genetically edited to become have reduced sensitivity to TGF-beta. TGF-beta is an inhibitor of NK cell function on at least the levels of proliferation and cytotoxicity. See, for example, FIG. 8A which schematically shows some of the inhibitory pathways by which TGF-beta reduces NK cell activity and/or proliferation. Thus, according to some embodiments, the expression of the TGF-beta receptor is knocked down or knocked out through gene editing, such that the edited NK is resistant to the immunosuppressive effects of TGF-beta in the tumor microenvironment. In several embodiments, the TGFB2 receptor is knocked down or knocked out through gene editing, for example, by use of CRISPR-Cas editing. Small interfering RNA, antisense RNA, TALENs or zinc fingers are used in other embodiments. Other isoforms of the TGF-beta receptor (e.g., TGF-beta 1 and/or TGF-beta 3) are edited in some embodiments. In some embodiments TGF-beta receptors in T cells are knocked down through gene editing. Non-limiting examples of guide RNAs that can target an endonuclease, such as Cas9, to edit a TGFBR2 gene are provided in Table 3, below (additional information on TGFBR editing can be found, for example in International Patent Application No. PCT/US2020/035752, which is incorporated in its entirety by reference herein).

TABLE 3
TGFb Receptor Type 2 Isoform Guide RNAs

| SEQ ID NO: | Name | Sequence | Target |
|---|---|---|---|
| 34 | TGFBR2-1 | CCCCTACCATGACTTTATTC | Exon 4 |
| 35 | TGFBR2-2 | ATTGCACTCATCAGAGCTAC | Exon 4 |
| 36 | TGFBR2-3 | AGTCATGGTAGGGGAGCTTG | Exon 4 |
| 37 | TGFBR2-4 | TGCTGGCGATACGCGTCCAC | Exon 1 |
| 38 | TGFBR2-5 | GTGAGCAATCCCCCGGGCGA | Exon 4 |
| 39 | TGFBR2-6 | AACGTGCGGTGGGATCGTGC | Exon 1 |

In several embodiments, genetic editing (whether knock out or knock in) of any of the target genes (e.g., CISH, TGFBR2, or any other target gene disclosed in International Patent Application No. PCT/US2020/035752, U.S. Provisional Application No. 63/121,206, or U.S. Provisional Application No. 63/201,159, each of which is incorporated by reference herein in its entirety), is accomplished through targeted introduction of DNA breakage, and subsequent DNA repair mechanism. In several embodiments, double strand breaks of DNA are repaired by non-homologous end joining (NHEJ), wherein enzymes are used to directly join the DNA ends to one another to repair the break. In several embodiments, however, double strand breaks are repaired by homology directed repair (HDR), which is advantageously more accurate, thereby allowing sequence specific breaks and repair. HDR uses a homologous sequence as a template for regeneration of missing DNA sequences at the break point, such as a vector with the desired genetic elements (e.g., an insertion element to disrupt the coding sequence of a TCR) within a sequence that is homologous to the flanking sequences of a double strand break. This will result in the desired change (e.g., insertion) being inserted at the site of the DSB.

In several embodiments, gene editing is accomplished by one or more of a variety of engineered nucleases. In several embodiments, restriction enzymes are used, particularly when double strand breaks are desired at multiple regions. In several embodiments, a bioengineered nuclease is used. Depending on the embodiment, one or more of a Zinc Finger Nuclease (ZFN), transcription-activator like effector nuclease (TALEN), meganuclease and/or clustered regularly interspaced short palindromic repeats (CRISPR/Cas9) system are used to specifically edit the genes encoding one or more of the TCR subunits.

Meganucleases are characterized by their capacity to recognize and cut large DNA sequences (from 14 to 40 base pairs). In several embodiments, a meganuclease from the LAGLIDADG family is used, and is subjected to mutagenesis and screening to generate a meganuclease variant that recognizes a unique sequence(s), such as a specific site in the TCR, or CISH, or any other target gene disclosed herein. Target sites in the TCR can readily be identified. Further information of target sites within a region of the TCR can be found in US Patent Publication No. 2018/0325955, and US Patent Publication No. 2015/0017136, each of which is incorporated by reference herein in its entirety. In several embodiments, two or more meganucleases, or functions fragments thereof, are fused to create a hybrid enzymes that recognize a desired target sequence within the target gene (e.g., CISH).

In contrast to meganucleases, ZFNs and TALEN function based on a non-specific DNA cutting catalytic domain which is linked to specific DNA sequence recognizing peptides such as zinc fingers or transcription activator-like effectors (TALEs). Advantageously, the ZFNs and TALENs thus allow sequence-independent cleavage of DNA, with a high degree of sequence-specificity in target recognition. Zinc finger motifs naturally function in transcription factors to recognize specific DNA sequences for transcription. The C-terminal part of each finger is responsible for the specific recognition of the DNA sequence. While the sequences recognized by ZFNs are relatively short, (e.g., ˜3 base pairs), in several embodiments, combinations of 2, 3, 4, 5, 6, 7, 8, 9, 10 or more zinc fingers whose recognition sites have been characterized are used, thereby allowing targeting of specific sequences, such as a portion of the TCR (or an immune checkpoint inhibitor). The combined ZFNs are then fused with the catalytic domain(s) of an endonuclease, such as Fokl (optionally a Fokl heterodimer), in order to induce a targeted DNA break. Additional information on uses of ZFNs to edit the TCR and/or immune checkpoint inhibitors can be found in U.S. Pat. No. 9,597,357, which is incorporated by reference herein.

Transcription activator-like effector nucleases (TALENs) are specific DNA-binding proteins that feature an array of 33 or 34-amino acid repeats. Like ZFNs, TALENs are a fusion of a DNA cutting domain of a nuclease to TALE domains, which allow for sequence-independent introduction of double stranded DNA breaks with highly precise target site recognition. TALENs can create double strand breaks at the target site that can be repaired by error-prone non-homologous end-joining (NHEJ), resulting in gene disruptions through the introduction of small insertions or deletions. Advantageously, TALENs are used in several embodiments, at least in part due to their higher specificity in DNA binding, reduced off-target effects, and ease in construction of the DNA-binding domain.

CRISPRs (Clustered Regularly Interspaced Short Palindromic Repeats) are genetic elements that bacteria use as protection against viruses. The repeats are short sequences that originate from viral genomes and have been incorporated into the bacterial genome. Cas (CRISPR associated proteins) process these sequences and cut matching viral DNA sequences. By introducing plasmids containing Cas genes and specifically constructed CRISPRs into eukaryotic cells, the eukaryotic genome can be cut at any desired position. Additional information on CRISPR can be found in US Patent Publication No. 2014/0068797, which is incorporated by reference herein. In several embodiments, CRISPR is used to manipulate the gene(s) encoding a target gene to be knocked out or knocked in, for example CISH, TGFBR2, TCR, B2M, CIITA, CD47, HLA-E, etc. In several embodiments, CRISPR is used to edit one or more of the TCRs of a T cell and/or the genes encoding one or more immune checkpoint inhibitors. In several embodiments, the immune checkpoint inhibitor is selected from one or more of CTLA4 and PD1. In several embodiments, CRISPR is used to truncate one or more of TCRa, TCRB, TCRy, and TCRo. In several embodiments, a TCR is truncated without impacting the function of the CD3z signaling domain of the TCR. Depending on the embodiment and which target gene is to be edited, a Class 1 or Class 2 Cas is used. In several embodiments, a Class 1 Cas is used and the Cas type is selected from the following types: I, IA, IB, IC, ID, IE, IF, IU, III, IIIA, IIIB, IIIC, IIID, IV IVA, IVB, and combinations thereof. In several embodiments, the Cas is selected from the group consisting of Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, a Class 2 Cas is used and the Cas type is selected from the following types: II, IIA, IIB, IIC, V, VI, and combinations thereof. In several embodiments, the Cas is selected from the group consisting of Cas9, Csn2, Cas4, Cpf1, C2c1, C2c3, Cas13a (previously known as C2c2), Cas13b, Cas13c, CasX, CasY and combinations thereof. In some embodiments, class 2 CasX is used, wherein CasX is capable of forming a complex with a guide nucleic acid and wherein the complex can bind to a target DNA, and wherein the target DNA comprises a non-target strand and a target strand. In some embodiments, class 2 CasY is used, wherein CasY is capable of binding and modifying a target nucleic acid and/or a polypeptide associated with target nucleic acid.

In several embodiments, as discussed above, editing of CISH advantageously imparts to the edited cells, particularly edited NK cells, enhanced expansion, cytotoxicity and/or persistence. Additionally, in several embodiments, the modification of the TCR comprises a modification to TCRa, but without impacting the signaling through the CD3 complex, allowing for T cell proliferation. In one embodiment, the TCRa is inactivated by expression of pre-Ta in the cells, thus restoring a functional CD3 complex in the absence of a functional alpha/beta TCR. As disclosed herein, the non-alloreactive modified T cells are also engineered to express a CAR to redirect the non-alloreactive T cells specificity towards tumor marker, but independent of MHC. Combinations of editing are used in several embodiments, such as knockout of the TCR and CISH in combination, or knock out of CISH and knock in of CD47, by way of non-limiting examples. In several embodiments, the gene edit to reduce/eliminate expression of, for example, CISH, is performed prior to expanding the cells in culture. For example, in several embodiments, the cells to be expanded are edited at least 12 hours, at least 18 hours, at least 24 hours, at least 36 hours, or at least 48 hours prior to expansion.

### Cells to Facilitate Expansion of Immune Cells

In several embodiments, cell lines are used in a co-culture with a population of immune cells that are to be expanded. Such cell lines are referred to herein as “stimulatory cells,” which can also be referred to as “feeder cells”. In several embodiments, the entire population of immune cells is to be expanded, while in several embodiments, a selected immune cell subpopulation is to be expanded. For example, in several embodiments, NK cells are expanded relative to other immune cell subpopulations (such as T cells). In other embodiments, both NK cells and T cells are expanded. In several embodiments, the feeder cells are themselves genetically modified. In some embodiments, the feeder cells do not express MHC I molecules, which have an inhibitory effect on NK cells. In some embodiments, the feeder cells need not entirely lack MHC I expression, however they may express MHC I molecules at a lower level than a wild type cell. For example, in several embodiments, if a wild type cell expresses an MHC at a level of X, the cell lines used may express MHC at a level less than 95% of X, less than 90% of X, less than 85% of X, less than 80% of X, less than 70% of X, less than 50% of X, less than 25% of X, and any expression level between (and including) those listed. In several embodiments, the stimulatory cells are immortalized, e.g., a cancer cell line. However, in several embodiments, the stimulatory cells are primary cells.

Various cell types can be used as feeder cells, depending on the embodiment. These include, but are not limited to, K562 cells, certain Wilm's Tumor cell lines (for example Wilms tumor cell line HFWT), endometrial tumor cells (for example, HHUA), melanoma cells (e.g., HMV-II), hepatoblastoma cells (e.g., HuH-6), lung small cell carcinoma cells (e.g., Lu-130 and Lu-134-A), neuroblastoma cells (e.g., NB19 and NB69), embryonal carcinoma testis cells (e.g., NEC14), cervical carcinoma cells (TCO-2), neuroblastoma cells (e.g., TNB1), 721.221 EBV transformed B cell line, among others.

In additional embodiments, the feeder cells also have reduced (or lack) MHC II expression, as well as having reduced (or lacking) MHC I expression. In some embodiments, other cell lines that may initially express MHC class I molecules can be used, in conjunction with genetic modification of those cells to reduce or knock out MHC I expression. Genetic modification can be accomplished through the use of gene editing techniques (e.g. a Crispr/Cas system; RNA editing with an Adenosine deaminases acting on RNA (ADAR), zinc fingers, TALENS, etc.), inhibitory RNA (e.g., SIRNA), or other molecular methods to disrupt and/or reduce the expression of MHC I molecules on the surface of the cells.

As discussed in more detail below, in several embodiments, the feeder cells are engineered to express certain stimulatory molecules (e.g. interleukins, CD3, 4-1BBL, etc.) to promote immune cell expansion and activation. Engineered feeder cells are disclosed in, for example, International Patent Application PCT/SG2018/050138, which is incorporated in its entirety by reference herein. In several embodiments, the stimulatory molecules, such as interleukin 12, 18, and/or 21 are separately added to the co-culture media, for example at defined times and in particular amounts, to effect an enhanced expansion of a desired sub-population(s) of immune cells.

### Stimulatory Molecules to Facilitate Expansion of Immune Cells

As discussed briefly above, certain molecules promote the expansion of immune cells, such as NK cells or T cells, including engineered NK or T cells, and also cells that have optionally been genetically edited. Depending on the embodiment, the stimulatory molecule, or molecules, can be expressed on the surface of the feeder cells used to expand the immune population. For example, in several embodiments a K562 feeder cell population is engineered to express 4-1BBL and/or membrane bound interleukin 15 (mbIL15). Additional embodiments relate to further membrane bound interleukins or stimulatory agents. Examples of such additional membrane bound stimulatory molecules can be found in International Patent Application PCT/SG2018/050138 and additional information on stimulating agents can be found in International Patent Application No. PCT/US2020/044033, each of which is incorporated in its entirety by reference herein.

In several embodiments, the methods disclosed herein relate to addition of one or more stimulatory molecules to the culture media in which engineered feeder cells and engineered NK cells are co-cultured. As discussed above, the cells may also be genetically edited. The editing and engineering may be performed in any order, however, in several embodiments, the cells are first edited, then subject to expansion for a period of time, with the engineering (e.g., to yield expression of a CAR) being performed during the expansion. In several embodiments, one or more interleukins is added. For example, in several embodiments, IL2 is added to the media. In several embodiments, IL12 is added to the media. In several embodiments, IL18 is added to the media. In several embodiments, IL21 is added to the media. In several embodiments, combinations of two or more of IL2, IL12, IL18, and/or IL21 is added to the media. In some embodiments, rather than using a feeder cell with mbIL15, soluble IL15 is added to the media (alone or in combination with any of IL2, IL12, IL18, and IL21).

In several embodiments, the media comprises one or more vitamin, inorganic salt and/or amino acids. In several embodiments, the media comprises 1, 2, 3, 4, 5, 6, 7, 8, 9, 10 or all of Glycine, L-Arginine, L-Asparagine, L-Aspartic acid, L-Cystine (e.g., L-Cystine 2HCI), L-Glutamic Acid, L-Glutamine, L-Histidine, L-Hydroxyproline, L-Isoleucine, L-Leucine, L-Lysine hydrochloride, L-Methionine, L-Phenylalanine, L-Proline, L-Serine, L-Threonine L-Tryptophan, L-Tyrosine (e.g., L-Tyrosine disodium salt dehydrate), and L-Valine. In several embodiments, the media comprises 1, 2, 3, 4, or more of Biotin, Choline chloride, D-Calcium pantothenate, Folic Acid, i-Inositol, Niacinamide, Para-Aminobenzoic Acid, Pyridoxine hydrochloride, Riboflavin, Thiamine hydrochloride, and Vitamin B12. In several embodiments, the media comprises 1, 2, 3, 4, or more of Calcium nitrate (Ca(NO3) 2 4H2O), Magnesium Sulfate (MgSO4) (e.g., Magnesium Sulfate (MgSO4) (anhyd.)), Potassium Chloride (KCl), Sodium Bicarbonate (NaHCO₃), Sodium Chloride (NaCl), and Sodium Phosphate dibasic (Na2HPO4) (e.g., Sodium Phosphate dibasic (Na2HPO4) anhydrous).

In several embodiments, the media further comprises D-Glucose and/or glutathione (optionally reduced glutathione). In several embodiments, the media further comprises serum (e.g., fetal bovine serum) in an amount ranging from about 1% to about 20%. In several embodiments, the serum is heat-inactivated. In several embodiments, the media is serum-free. In several embodiments, the media is xenofree.

Depending on the embodiment, IL2 is used to supplement the culture media and enhance expansion, or other characteristics, of NK cells. In several embodiments, the concentration of IL2 used ranges from about 1 IU/mL to about 1000 IU/mL, including for example, about 1 IU/mL to about 5 IU/ml (e.g., 1, 2, 3, 4, and 5, about 5 IU/mL to about 10 IU/ml (e.g., 5, 6, 7, 8, 9, and 10), about 10 IU/mL to about 20 IU/mL (e.g., about 10, 12, 14, 16, 18, and 20), about 20 IU/mL to about 30 IU/mL (e.g., about 20, 22, 24, 26, 28, and 30), about 30 IU/mL to about 40 IU/ml (e.g., 30, 32, 34, 36, 38, and 40), about 40 to about 50 IU/mL (e.g., 40, 42, 44, 46, 48, 50), about 50 IU/mL to about 75 IU/mL (e.g., 50, 55, 60, 65, 70, and 75), about 75 IU/mL to about 100 IU/ml (e.g., 75, 80, 85, 90, 95, and 100), about 100 IU/mL to about 200 IU/mL (e.g., 100, 125, 150, 275, and 200), about 200 IU/mL to about 300 IU/mL (e.g., 200, 225, 250, 275, and 300), about 300 IU/mL to about 400 IU/ml (e.g., 300, 325, 350, 375, and 400), about 400 IU/mL to about 500 IU/ml (e.g., 400, 425, 450, 475, and 500), about 500 IU/mL to about 750 IU/mL (e.g., 500, 550, 600, 650, 700, and 750), or about 750 IU/mL to about 1000 IU/mL (e.g., 750, 800, 850, 900, 950, and 1000), and any concentration therebetween, including endpoints. In several embodiments, IL2 may be added at multiple time points during culture. In some such embodiments the concentration of IL2 used differs between selected time points.

Depending on the embodiment, IL12 (e.g., IL12A and/or IL12B) is used to supplement the culture media and enhance expansion, or other characteristics, of NK cells. In several embodiments, the concentration of IL12 (either IL12A or IL12B) used ranges from about 0.01 ng/ml to about 100 ng/ml, including, for example, about 0.01 ng/ml to about 0.05 ng/ml (e.g., 0.01, 0.02, 0.03, 0.04, and 0.05), about 0.05 ng/ml to about 0.1 ng/ml (e.g., 0.05, 0.06, 0.07, 0.08, 0.09 and 0.1), about 0.1 ng/ml to about 0.5 ng/ml (e.g., 0.1, 0.2, 0.3, 0.4, and 0.5), about 0.5 ng/ml to about 1.0 ng/ml (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/ml to about 5.0 ng/ml (e.g., 2.0, 3.0, 4.0, and 5.0), about 5.0 ng/ml to about 10.0 ng/ml (e.g., 5.0, 6.0, 7.0, 8.0, 9.0 and 10.0), about 10.0 ng/ml to about 15.0 ng/ml (e.g., 10.0, 11.0, 12.0, 13.0, 14.0, and 15.0), about 15.0 ng/ml to about 20.0 ng/mL (e.g., 15.0, 16.0, 17.0, 18.0, 19.0, and 20.0), about 20.0 ng/ml to about 25.0 ng/ml (e.g., 20.0, 21.0, 22.0, 23.0, 24.0, and 25.0), about 25.0 ng/ml to about 30.0 ng/ml (e.g., 25.0, 26.0, 27.0, 28.0, 29.0, and 30.0), about 30.0 ng/ml to about 50.0 ng/ml (e.g., 30.0, 35.0, 40.0, 45.0, and 50.0), about 50.0 ng/mL to about 75.0 ng/ml (e.g., 50.0, 55.0, 60.0, 65.0, 70.0, and 75.0), about 75.0 ng/ml to about 100.0 ng/ml (e.g., 75.0, 80.0, 85.0, 90.0, 95.0, and 100.0), and any concentration therebetween, including endpoints. In several embodiments, the concentration of IL12 is between about 0.01 ng/ml and about 8 ng/ml, including any concentration therebetween, including endpoints. In several embodiments, the concentration of IL12 is between about 0.01 ng/ml and about 1 ng/ml, including any concentration therebetween, including endpoints (and including other units of concentration, such as about 0.01 IU/mL to about 1.0 IU/mL, including about 0.5, about 0.6, about 0.7, about 0.8, about 0.9 IU/mL and values in between those listed).

In some embodiments, a mixture of IL12A and IL12B is used. In several embodiments, a particular ratio of IL12A:IL12B is used, for example, 1:10, 1:50, 1:100, 1:150, 1:200, 1:250:, 1:500, 1:1000, 1:10,000, 10,000:1, 1000:1, 500:1, 250:1, 150:1, 100:1, 10:1 and any ratio there between, including endpoints.

In some embodiments, interleukin 18 (IL18) is used to enhance expansion, or other characteristics, of NK cells. In several embodiments, the concentration of IL18 used ranges from about 0.01 ng/ml to about 100 ng/ml, including, for example, about 0.01 ng/mL to about 0.05 ng/ml (e.g., 0.01, 0.02, 0.03, 0.04, and 0.05), about 0.05 ng/mL to about 0.1 ng/mL (e.g., 0.05, 0.06, 0.07, 0.08, 0.09 and 0.1), about 0.1 ng/ml to about 0.5 ng/ml (e.g., 0.1, 0.2, 0.3, 0.4, and 0.5), about 0.5 ng/ml to about 1.0 ng/mL (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/ml to about 5.0 ng/ml (e.g., 2.0, 3.0, 4.0, and 5.0), about 5.0 ng/ml to about 10.0 ng/ml (e.g., 5.0, 6.0, 7.0, 8.0, 9.0 and 10.0), about 10.0 ng/ml to about 15.0 ng/ml (e.g., 10.0, 11.0, 12.0, 13.0, 14.0, and 15.0), about 15.0 ng/ml to about 20.0 ng/ml (e.g., 15.0, 16.0, 17.0, 18.0, 19.0, and 20.0), about 20.0 ng/ml to about 25.0 ng/ml (e.g., 20.0, 21.0, 22.0, 23.0, 24.0, and 25.0), about 25.0 ng/ml to about 30.0 ng/ml (e.g., 25.0, 26.0, 27.0, 28.0, 29.0, and 30.0), about 30.0 ng/ml to about 50.0 ng/ml (e.g., 30.0, 35.0, 40.0, 45.0, and 50.0), about 50.0 ng/ml to about 75.0 ng/ml (e.g., 50.0, 55.0, 60.0, 65.0, 70.0, and 75.0), about 75.0 ng/ml to about 100.0 ng/ml (e.g., 75.0, 80.0, 85.0, 90.0, 95.0, and 100.0), and any concentration therebetween, including endpoints.

In some embodiments interleukin 21 (IL21) is used to enhance expansion, or other characteristics, of NK cells. In several embodiments, the concentration of IL21 used ranges from about 0.01 ng/ml to about 100 ng/ml, including, for example, about 0.01 ng/ml to about 0.05 ng/ml (e.g., 0.01, 0.02, 0.03, 0.04, and 0.05), about 0.05 ng/ml to about 0.1 ng/ml (e.g., 0.05, 0.06, 0.07, 0.08, 0.09 and 0.1), about 0.1 ng/mL to about 0.5 ng/ml (e.g., 0.1, 0.2, 0.3, 0.4, and 0.5), about 0.5 ng/ml to about 1.0 ng/ml (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/ml to about 5.0 ng/ml (e.g., 2.0, 3.0, 4.0, and 5.0), about 5.0 ng/ml to about 10.0 ng/ml (e.g., 5.0, 6.0, 7.0, 8.0, 9.0 and 10.0), about 10.0 ng/ml to about 15.0 ng/ml (e.g., 10.0, 11.0, 12.0, 13.0, 14.0, and 15.0), about 15.0 ng/ml to about 20.0 ng/ml (e.g., 15.0, 16.0, 17.0, 18.0, 19.0, and 20.0), about 20.0 ng/ml to about 25.0 ng/mL (e.g., 20.0, 21.0, 22.0, 23.0, 24.0, and 25.0), about 25.0 ng/ml to about 30.0 ng/ml (e.g., 25.0, 26.0, 27.0, 28.0, 29.0, and 30.0), about 30.0 ng/ml to about 50.0 ng/ml (e.g., 30.0, 35.0, 40.0, 45.0, and 50.0), about 50.0 ng/ml to about 75.0 ng/ml (e.g., 50.0, 55.0, 60.0, 65.0, 70.0, and 75.0), about 75.0 ng/ml to about 100.0 ng/ml (e.g., 75.0, 80.0, 85.0, 90.0, 95.0, and 100.0), and any concentration therebetween, including endpoints.

In some embodiments interleukin 15 (IL15) is used in a soluble format (either in place of, or in addition to mbIL15 on the feeder cells) to enhance expansion, or other characteristics, of NK cells. In several embodiments, the concentration of IL15 used ranges from about 0.01 ng/ml to about 100 ng/ml, including, for example, about 0.01 ng/ml to about 0.05 ng/ml (e.g., 0.01, 0.02, 0.03, 0.04, and 0.05), about 0.05 ng/ml to about 0.1 ng/ml (e.g., 0.05, 0.06, 0.07, 0.08, 0.09 and 0.1), about 0.1 ng/ml to about 0.5 ng/ml (e.g., 0.1, 0.2, 0.3, 0.4, and 0.5), about 0.5 ng/ml to about 1.0 ng/ml (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/ml to about 5.0 ng/ml (e.g., 2.0, 3.0, 4.0, and 5.0), about 5.0 ng/ml to about 10.0 ng/ml (e.g., 5.0, 6.0, 7.0, 8.0, 9.0 and 10.0), about 10.0 ng/ml to about 15.0 ng/ml (e.g., 10.0, 11.0, 12.0, 13.0, 14.0, and 15.0), about 15.0 ng/ml to about 20.0 ng/ml (e.g., 15.0, 16.0, 17.0, 18.0, 19.0, and 20.0), about 20.0 ng/ml to about 25.0 ng/ml (e.g., 20.0, 21.0, 22.0, 23.0, 24.0, and 25.0), about 25.0 ng/ml to about 30.0 ng/ml (e.g., 25.0, 26.0, 27.0, 28.0, 29.0, and 30.0), about 30.0 ng/ml to about 50.0 ng/ml (e.g., 30.0, 35.0, 40.0, 45.0, and 50.0), about 50.0 ng/ml to about 75.0 ng/ml (e.g., 50.0, 55.0, 60.0, 65.0, 70.0, and 75.0), about 75.0 ng/ml to about 100.0 ng/ml (e.g., 75.0, 80.0, 85.0, 90.0, 95.0, and 100.0), and any concentration therebetween, including endpoints.

In some embodiments interleukin 22 (IL22) is used to facilitate expansion of NK cells. In several embodiments, the concentration of IL22 used ranges from about 0.01 ng/ml to about 100 ng/ml, including, for example, about 0.01 ng/ml to about 0.05 ng/ml (e.g., 0.01, 0.02, 0.03, 0.04, and 0.05), about 0.05 ng/ml to about 0.1 ng/ml (e.g., 0.05, 0.06, 0.07, 0.08, 0.09 and 0.1), about 0.1 ng/ml to about 0.5 ng/ml (e.g., 0.1, 0.2, 0.3, 0.4, and 0.5), about 0.5 ng/ml to about 1.0 ng/ml (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/ml to about 5.0 ng/ml (e.g., 2.0, 3.0, 4.0, and 5.0), about 5.0 ng/ml to about 10.0 ng/ml (e.g., 5.0, 6.0, 7.0, 8.0, 9.0 and 10.0), about 10.0 ng/ml to about 15.0 ng/ml (e.g., 10.0, 11.0, 12.0, 13.0, 14.0, and 15.0), about 15.0 ng/mL to about 20.0 ng/ml (e.g., 15.0, 16.0, 17.0, 18.0, 19.0, and 20.0), about 20.0 ng/ml to about 25.0 ng/ml (e.g., 20.0, 21.0, 22.0, 23.0, 24.0, and 25.0), about 25.0 ng/ml to about 30.0 ng/ml (e.g., 25.0, 26.0, 27.0, 28.0, 29.0, and 30.0), about 30.0 ng/ml to about 50.0 ng/mL (e.g., 30.0, 35.0, 40.0, 45.0, and 50.0), about 50.0 ng/ml to about 75.0 ng/ml (e.g., 50.0, 55.0, 60.0, 65.0, 70.0, and 75.0), about 75.0 ng/ml to about 100.0 ng/mL (e.g., 75.0, 80.0, 85.0, 90.0, 95.0, and 100.0), and any concentration therebetween, including endpoints.

If two stimulatory agents are used, the relative ratio between the two can range from a ratio of 1:10, 1:20, 1:50, 1:100, 1:150, 1:200, 1:250, 1:500, 1:750, 1:1,000, 1:10,000, 1:50,000, 1:100,000, 100,000:1, 50,000:1, 10,000:1, 1,000:1, 750:1, 500:1, 250:1, 200:1, 150:1, 100:1, 50:1, 20:1, 10:1, and any ratio in between those listed, including endpoints. Likewise, if three, or more, agents are used, the ratio between those additional agents and the other agents can employ any of the aforementioned ratios.

As discussed in more detail below, depending on the embodiment, the stimulatory molecules may be added at a specific point (or points) during the expansion process, or can be added such that they are present as a component of the culture medium through the co-culture process.

Methods of Co-culture and Immune Cell Expansion

In some embodiments, NK cells isolated from a peripheral blood donor sample are co-cultured with K562 cells modified to express 4-1BBL and mbIL15. While other approaches involve the expression of other membrane-bound cytokines, the generation of a feeder cell with multiple stimulatory molecules can be difficult to generate (e.g., to achieve desired levels of expression of the various stimulatory molecule, expression at the right time during expansion, etc.). Thus, several embodiments disclosed herein relate to the supplementation of the culture media with particular concentrations of various stimulatory agents at particular times. In several embodiments, feeder cells are seeded into culture vessels and allowed to reach near confluence. Immune cells can then be added to the culture at a desired concentration, ranging, in several embodiments from about 0.5×10⁶ cells/cm2 to about 5×10⁶ cells/cm2, including any density between those listed, including endpoints.

In several embodiments, immune cells are separated from a peripheral blood sample. Thereafter, in several embodiments, the immune cells can be expanded together, or an isolated subpopulation of cells, such as NK cells, is used.

Thereafter, the NK cells are seeded with the feeder cells, and optionally one or more cytokines (either in the culture media or as an exogenous supplement) and cultured for a first period of time, for example about 6 hours, about 12 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, or for any time between those listed, including endpoints. Each exposure (e.g., co-culture) to fresh feeder cells is referred to herein as a pulse.

In several embodiments, during the expansion process, for example, after the first period of expansion, the expanded cells (e.g., NK cells) are transduced with an engineered construct, such as a chimeric antigen receptor. Any variety of chimeric antigen receptor can be expressed in the engineered cells, such as NK cells, including those described in International PCT Application PCT/US2018/024650, PCT/IB2019/000141, PCT/IB2019/000181, and/or PCT/US2020/020824, PCT/US2020035752, PCT/US2021/036879, or U.S. Provisional Application No. 63/220,842, each of which is incorporated in its entirety by reference herein.

In several embodiments, the expanding cells are pulsed again with fresh feeder cells and cultured for about 6 hours, about 12 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, or for any time between those listed, including endpoints. The cells can then optionally be separated into multiple aliquots and stored (e.g., cryopreserved as a master cell bank) from which future expansions can be performed. In several embodiments, generation of a master cell bank involves 1 to 3 or 1 to 4 pulses with feeder cells and co-culturing for a total time ranging from about 14 days to about 36 days.

In several embodiments, cells that have been expanded and engineered (and optionally gene edited) are pulsed at least one additional time and are cultured for a period of time, for example about 6 hours, about 12 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, or for any time between those listed, including endpoints. It shall be noted that certain data presented herein relates to viral expression of a chimeric receptor complex expressing an NKG2D ligand binding domain (e.g., NKX101) or CD19 (e.g., NK19-1 or NKX019). However, any suitable chimeric receptor or chimeric antigen receptor can be used. Cells may optionally be separated into additional aliquots and cryopreserved (e.g., as a working cell bank) from which further expansion can be performed. In several embodiments, generation of a working cell bank involves 1 to 3 or 1 to 4 pulses with feeder cells and co-culturing for a total time ranging from about 14 days to about 36 days.

In several embodiments, cells that have been expanded to the working cell bank are subjected to at least one additional pulse of feeder cells and are cultured for a period of time, for example about 6 hours, about 12 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 18 days or about 21 days, or for any time between those listed, including endpoints. In several embodiments, at the termination of this co-culture, the cells have been sufficiently expanded and are aliquoted into individual patient doses and stored (e.g., cryopreserved) until administration.

Supplementation of the media with one or more stimulatory agents, such as IL12 and/or IL18 can occur at any time during the culturing process. For example, one or more stimulatory agents can be added at the inception of culturing, for example at time point zero (e.g., inception of culture). The agent, or agents, can be added a second, third, fourth, fifth, or more times. Subsequent additions may, or may not, be at the same concentration as a prior addition. The interval between multiple additions can vary, for example a time interval of about 12 hours, about 24 hours, about 36 hours, about 48 hours, about 72 hours, or longer, and any time therebetween, including endpoints.

If multiple additions of a stimulatory agent are used, the concentrations of a first supplemental addition can be at the same or a different concentration than the second (and/or any supplemental addition). For example, in several embodiments, the addition of a stimulatory agent over multiple time points can ramp up, ramp down, stay constant, or vary across multiple, non-equivalent concentrations.

In several embodiments, certain ratios of feeder cells to cells to be expanded are used. For example, in several embodiments a feeder cell: “target” cell ratio of about 10:1 to about 2:1 is used, including, for example 9:1, 8:1, 7:1, 6:1, 5:1, 4:1, 3:1 and any ratio therebetween, including endpoints. In several embodiments, 1:1 ratios are used, while in additional embodiments, can range from about: 1:10, 1:20, 1:50, 1:100, 1:1,000, 1:10,000, 1:50,000, 1:100,000, 100,000:1, 50,000:1, 10,000:1, 1,000:1, 100:1, 50:1, 20:1, 10:1, and any ratio in between those listed, including endpoints. In several embodiments, different feeder:target ratios are used at different pulses. In several embodiments, the degree of expansion is such that the resulting population is expanded by at least about 1000-fold, about 5000-fold, about 10,000-fold, about 50,000-fold, about 100,000-fold, about 500,000-fold, about 1 million-fold, about 2 million-fold, about 5 million-fold, about 20 million-fold, about 50 million-fold, about 100 million-fold, about 200 million-fold, about 500 million fold, about 800 million-fold, about 1 billion-fold, about 2 billion-fold or more (or any amount between those listed).

### EXAMPLES

The materials and methods disclosed in the Examples are non-limiting examples of materials and methods (including reagents and conditions) applicable to various embodiments provided in the present application.

### Example 1—Donor Expansion and Cytotoxicity Ranking

As discussed above, in several embodiments, a candidate donor is screened for cells that exhibit qualities that render the donor a preferred donor, whether that be potential for expansion or potentially enhanced cytotoxicity. In this non-limiting example, twelve donors where screened for their KIR profiles, as discussed above, and their expansion capacity and cytotoxicity after being expanded according to the expansion methods disclosed herein. As a non-limiting example, these donor NK cells were engineered to express an anti-CD19 CAR construct, for which additional information can be found in International Patent Application No. PCT/US2020/020824, the entire contents of which is incorporated by reference herein.

FIG. 1B shows data related to the expansion profile of NK cells from twelve donors after engineering to express an anti-CD19 CAR (or untransduced control) and expanded using the IL12/IL18 multiple pulse expansion methods disclosed herein, or without IL12/18. FIG. 1C shows corresponding cytotoxicity data. As can be seen in FIG. 1B, the presence or absence of IL12/IL18 in the culture process did not significantly impact expansion of the NK cells (whether expressing a CAR or not). Firstly, as shown in FIG. 1C, the transduction of the NK cells with the anti-CD19 CAR enhances the cytotoxicity profile of the NK cells, and notably, the use of IL12/IL18 in the expansion process further, and significantly, increased the cytotoxicity of the NK cells (against NALM6 tumor cells at a 1:4 E:T ratio at 96 hrs post-transduction. This data suggests that donor cells can obtain enhanced characteristics through the culturing process and based on their ability to respond to stimulatory cytokines in the culture process.

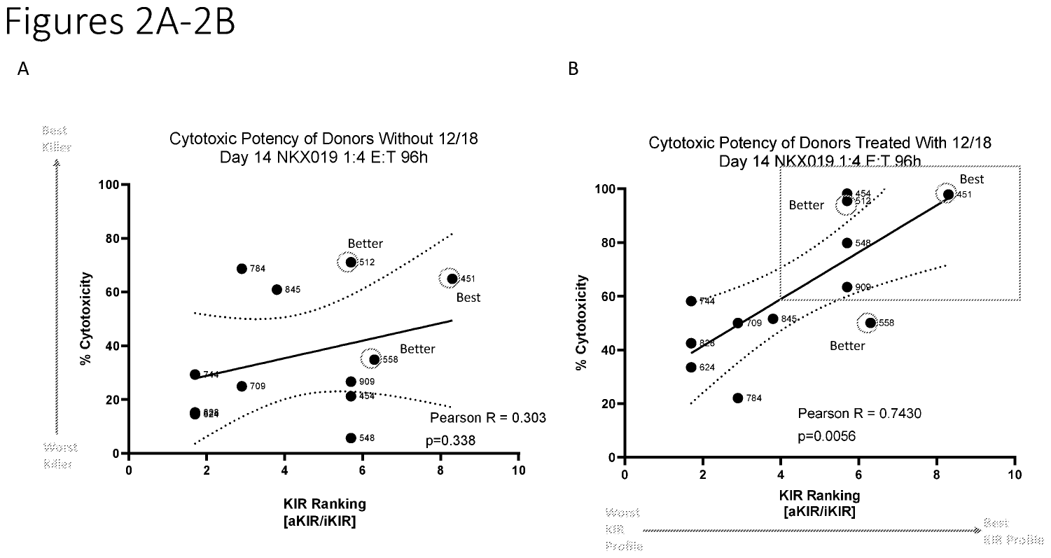

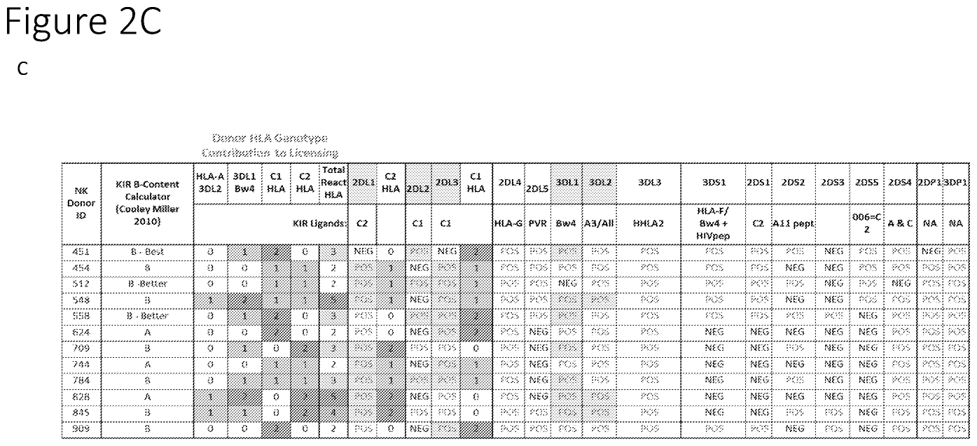

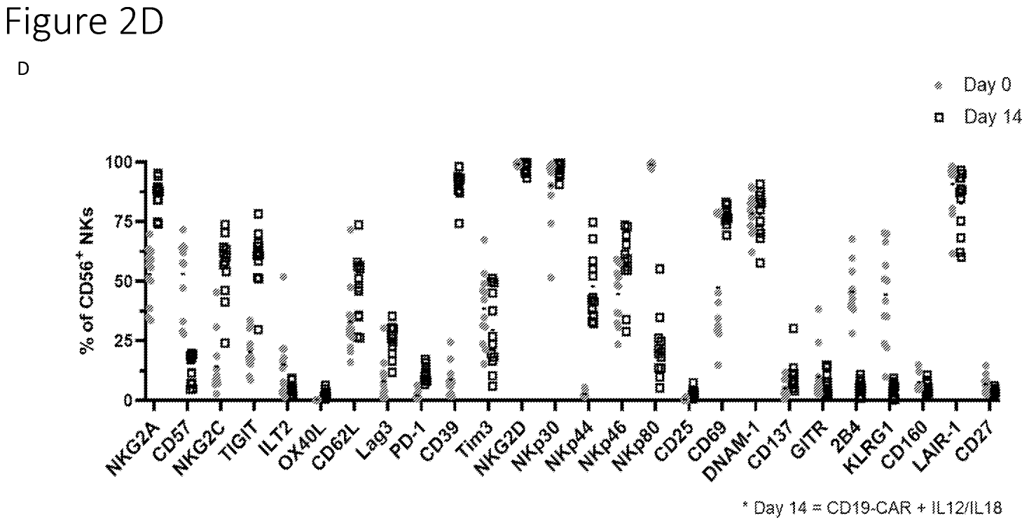

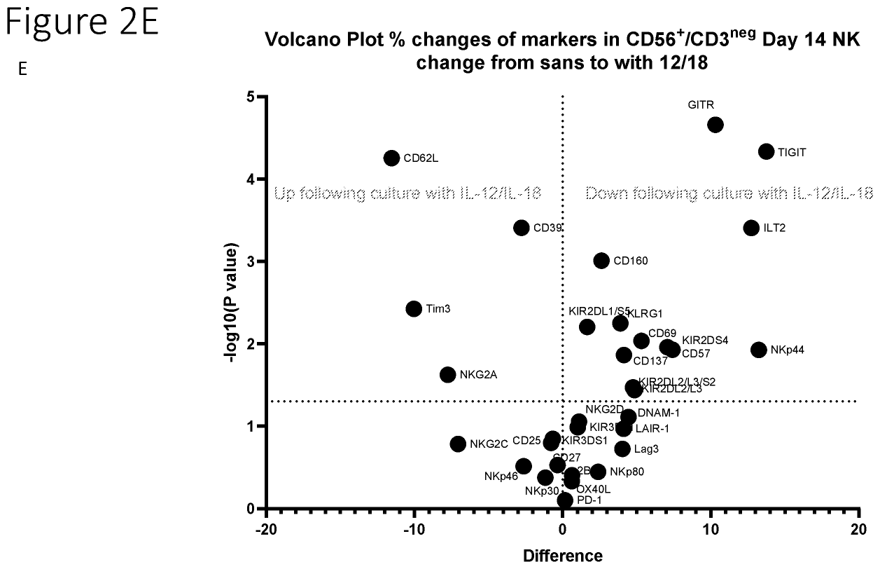

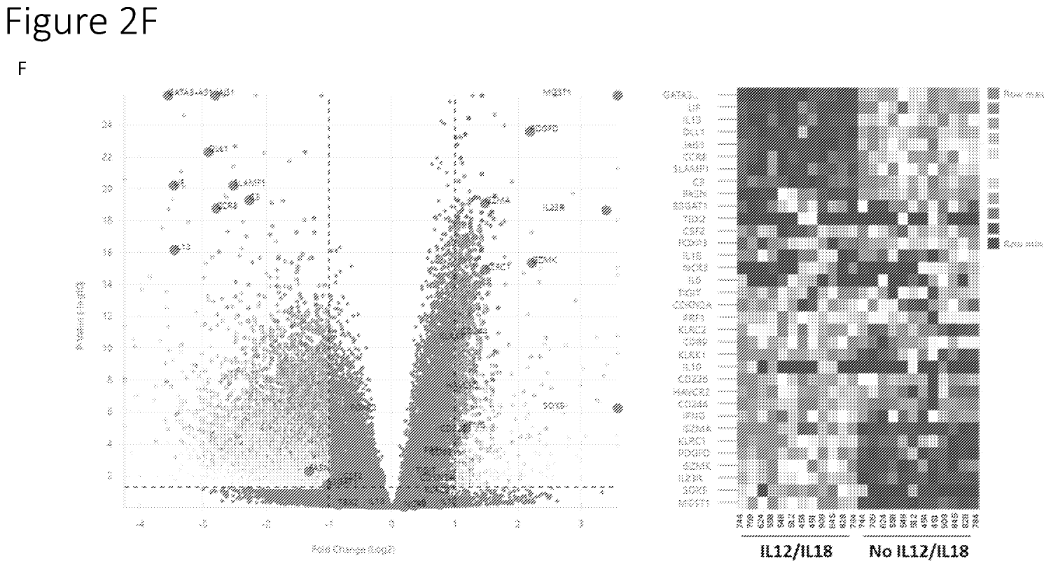

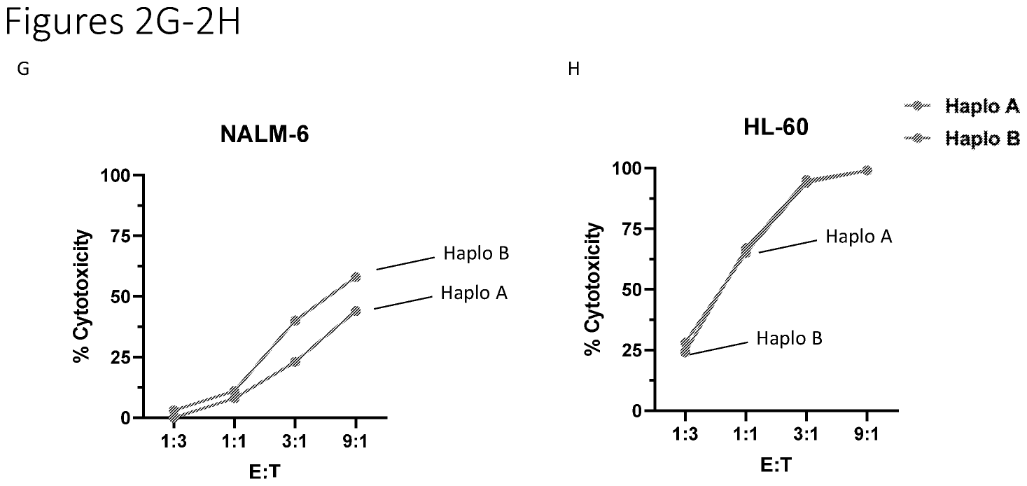

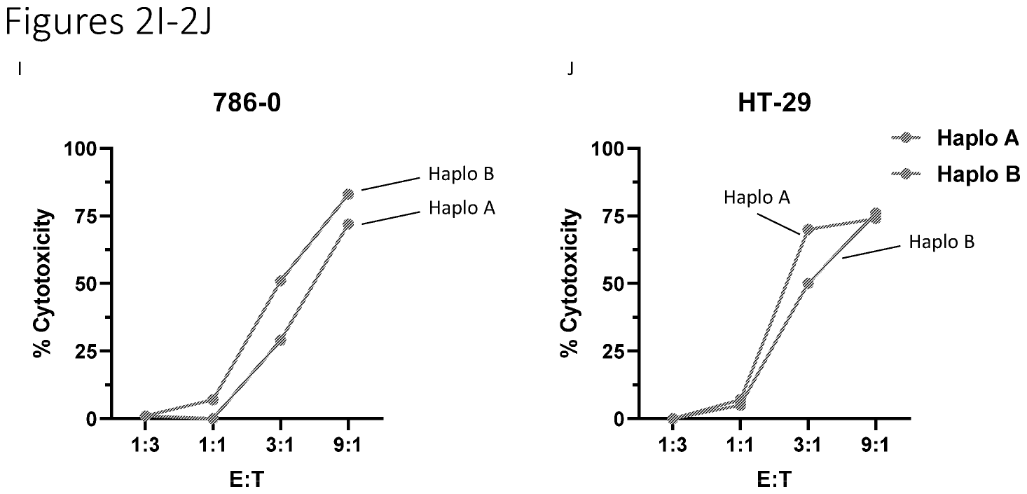

In view of the desire to screen candidate donors to identify preferred donors, the KIR profile of the donor cells was evaluated, according to methods disclosed herein. The data in FIG. 2A shows a correlation between the percent cytotoxicity exhibited and the total KIR haplotype of each donor (e.g., aKIR:iKIR ratio) when expanded without the use of IL12/IL18. FIG. 2B shows corresponding data when cells were expanded with IL12/IL18. Certain donors were identified in the data in FIG. 2A based on their performance in terms of cytotoxicity and “best” KIR profile. It is notable that these three donors maintained these categorizations when cultured with IL12/IL18, while also showing greatly increased cytotoxicity. The data in FIG. 2B demonstrate a highly significant correlation between the KIR profile and the cytotoxicity exhibited, which in several embodiments, allows for a candidate donor to be classified as a preferred donor based on their total KIR profile. To further characterize donor cells, a DNA-based high-resolution genotypic analysis of 12 donor cells (those from 2A and 2B) was undertaken. The analysis focused on the assessment of HLA & KIR genotype and also the KIR B content group was determined using an existing KIR Immuno-Polymorphism database (IPD-KIR). The analysis is summarized in FIG. 2C.

Further investigating the impact of expansion in the presence or absence of IL12 and IL18, NK cells from the twelve donors were genetically modified to express an anti-CD19-CAR-mbIL15 construct by retroviral transduction and expanded on K562 cells modified to express mbIL15 and 4-1BBL with or without soluble IL12/IL18 cytokines. Cells were characterized by flow cytometry on Day 0 & 14. These data show that the genetically modified NKs exhibit increased expression levels of activation markers, including activating NK receptors, for example TIGIT, Lag3, CD69, NKp30, NKp44, NKp46. Moreover, this assessment indicated a notable increase in NK memory associated markers, such as NKG2C, NKG2A, CD69. Additionally, there was a decrease in expression of the terminal differentiation marker CD57 by these expanded NKs. FIG. 2E shows a volcano plot of the changes detected in various NK cell markers at 14 days of expansion with, or without, IL12 and IL18. The upper left quadrant shows those markers that were increased after culture with IL12 and IL18, while the upper right quadrant shows those markers that were downregulated after culture with IL12 and IL18. FIG. 2F furthers this investigation into the impact of IL12 and IL18 on NK cells by evaluating expression of the genes encoding various markers of NK cell function by RNA sequencing (RNAseq). The additional of IL12 and IL18 to NK expansion cultures drives upregulation of genes associated with activation of NK cells. These data provide further evidence supporting the ability of culture conditions, including various cytokines, timings, feeder cells, and other variables disclosed herein, to impact the resultant expanded cells, not only in terms of raw cell numbers, post-expansion, but activation of the cells, and, as discussed herein, cytotoxic function and/or persistence of the expanded cells.

FIGS. 2G-2J continue the analysis of the interplay between donor identification leading to preferred, or even ideal, donor cells and the further enhancements that expansion conditions as provided for herein, which lead to further enhancements to NK cell cytotoxicity and persistence. There are two KIR primary haplotypes that can be found in humans: the group A haplotype and the group B haplotype. The group A haplotype has a fixed number of genes encoding inhibitory KIRs (with the exception of the activating receptor KIR2DS4). The group B haplotype has variable gene content, is generally more enriched in genes encoding activating receptors, and contains 1 or more of the following B-specific genes: KIR2DS1, KIR2DS2, KIR2DS3, KIR2DS5, KIR2DL2, and KIR2DL5. Certain studies in which pediatric acute lymphoblastic leukemia patients received human leukocyte antigen-haploidentical transplantation of ex vivo T-cell-depleted peripheral blood stem cells initially concluded that patients receiving transplanted cells from KIR haplotype B donors had an increased event-free survival. Unmodified KIR haplotype B donor NKs were treated with IL12/IL18 during expansion. These cells, even without genetic engineering to express a CAR, were able to effectively kill tumor cells across different tumor types, including NALM-6-B cell leukemia (2G), HL-60—myeloid cell leukemia (2H), 786-0—renal cancer (21), and HT-29—colorectal adenocarcinoma (2J). 6 donors were tested: 2 haplotype A and 4 haplotype B, group averages are shown. These data show that, despite the possibility that a starting KIR B haplotype is preferred in some instances, whether the donor cells are KIR haplotype A or B, the use of IL12 and IL18 during expansion imparts to both KIR haplotypes an ability to exhibit cytotoxic effects against a variety of target cells. In some instances where the exhibited cytotoxicity is lower than desired, cells can be further manipulated (e.g., engineered to express a CAR or other modification to synergistically increase the cytotoxicity and/or persistence of the resultant NK cells.

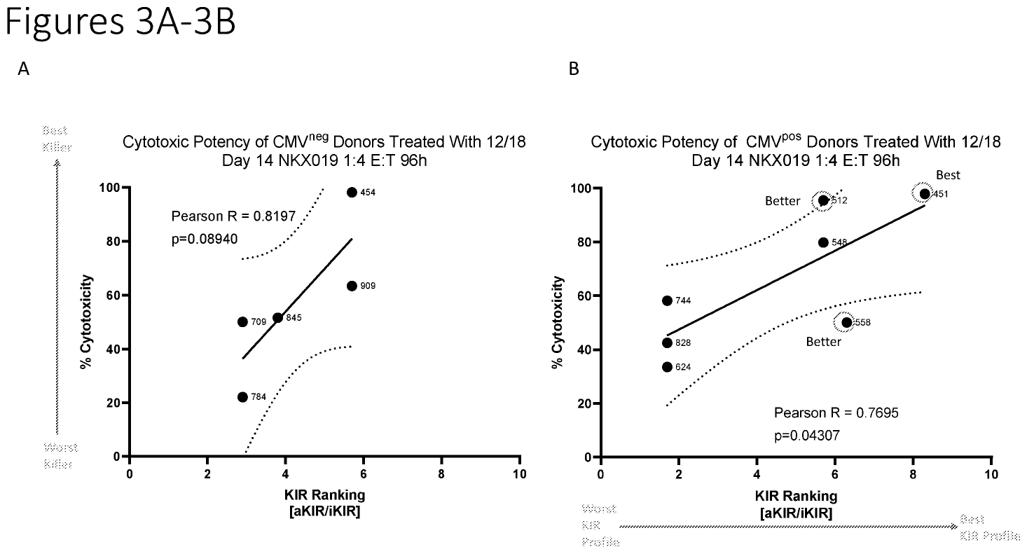

Despite the potential impact of a prior cytomegalovirus infection to impart to immune cells, such as NK cells, an enhanced cytotoxicity due to induction of a memory or memory-like phenotype, FIGS. 3A and 3B demonstrate that the correlation of donor potency and KIR profile is not impacted by CMV status. FIG. 3A shows the cytotoxicity: KIR profile correlation for CMV-negative donors and FIG. 3B shows the same data for CMV-positive donors. While the indicated donor cells who were, at least in this non-limiting example experiment, the highest performers and were CMV-positive, FIG. 3B shows lower performers in the CMV-positive group, while FIG. 3A shows high cytotoxicity-exhibiting cells in the CMV-negative group.

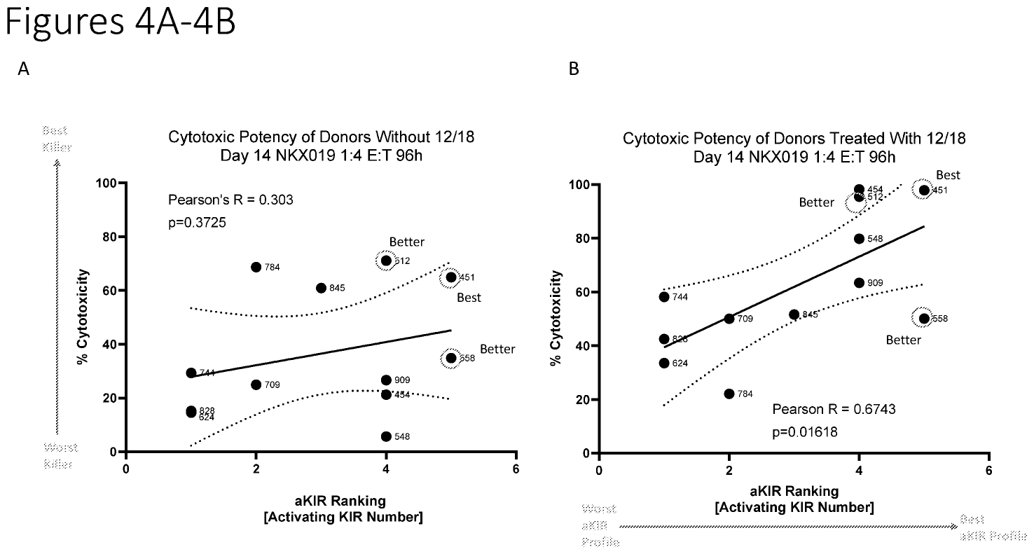

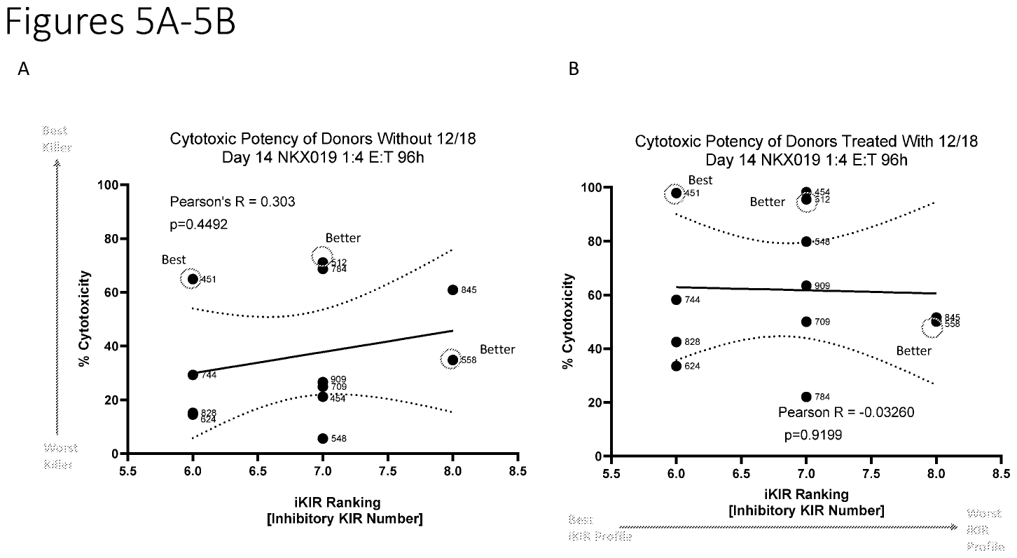

In order to more fully elucidate the assessment of the KIR profile of a donor on the predicted cytotoxicity of that donor's cells, correlations were performed that involve the cytotoxicity versus the activating KIR profile (as opposed to the total profile). These data are shown in FIGS. 4A-4B. FIG. 4A shows the cytotoxicity-activating KIR correlation for donors whose cells were expanded without IL12/IL18, while FIG. 4B shows the data related to expansion with IL12/IL18. As can be seen, there is still a significant correlation between the cytotoxicity of the cells expanded with IL12/IL18 and the activating KIR profile, but it is not as significant as when the total KIR profile is evaluated. However, according to some embodiments, assessment of the activating KIR profile is sufficient to categorize a donor as a preferred donor and move their cells into the expansion processes disclosed herein employing IL12/IL18. In contrast, FIGS. 5A-5B show cytotoxicity data as a function of iKIR ranking without (5A) or with (5B) IL12/IL18 used in the expansion process. As can be seen from the data, whether or not IL12/IL18 were used in the expansion process, the assessment of only the iKIRs present on the NK cells does not correlate with the ultimate cytotoxicity (despite the pro-cytotoxicity impact IL12/IL18 provide). Thus, in several embodiments, assessment of candidate donors relies on at least the evaluation of the aKIR profile, and in some embodiments, both the aKIR and iKIR profiles are determined.

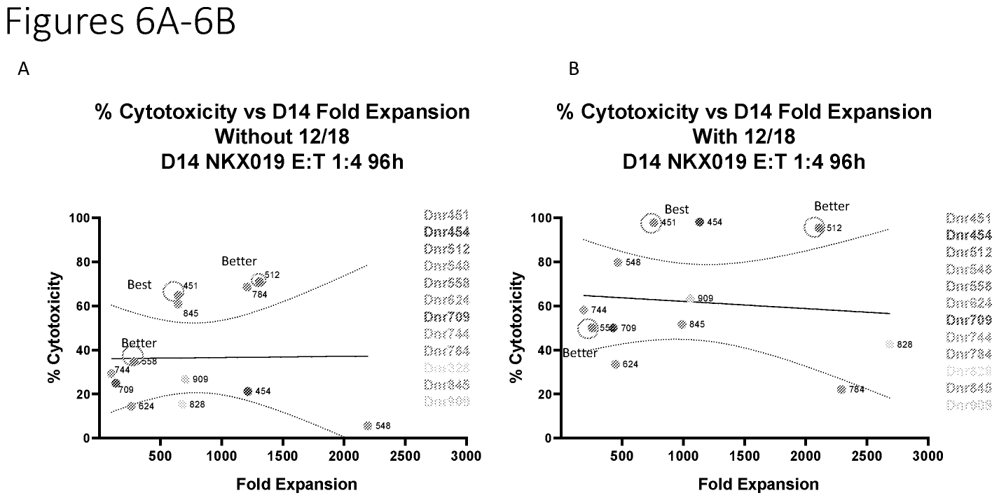

Further evaluation into the relationship between potential for expansion and cytotoxicity was undertaken. FIGS. 6A-6B show the relationship between cytotoxicity and fold expansion of cells without (6A) and with (6B) IL12/IL18 used in the expansion process. These data indicate that, for a given donor, whether IL12/IL18 are used in the expansion process or not, the ability of a given donor's cells to expand in culture is independent of the cytotoxicity towards tumor cells of those expanded donor cells. For example, cells from donor 828 exhibited about 600-700 fold expansion in the absence of IL12/IL18 and less than 20% cytotoxicity. When cultured with IL12/IL18, cells from donor 828 were expanded over 2500 fold, but increased in cytotoxicity to about only 40%. Cells from donor 451 exhibited about 600-700 fold expansion under either condition, but with the use of IL12/IL18 in the expansion, increased from just over 60% cytotoxicity to nearly 100% cytotoxicity. As another example, cell from donor 512 exhibited enhanced expansion and cytotoxicity in the presence of IL12/IL18. Thus, according to some embodiments, the ability of a donor cell to be expanded robustly in culture does not necessarily mean that those cells will be effective at eliminating tumor cells. However, as discussed above, evaluation of aKIR/iKIR does, in several embodiments, allow a prediction of future cytotoxicity. In several embodiments, the expansion process allows several hundred-fold expansion (e.g., at least about 100-fold, about 200-fold, about 300-fold, about-400 fold, about 500-fold, about 600-fold, about 700-fold, about 800-fold, about 900-fold, about 1000-fold, about 1500-fold, about 200-fold, or more (including amounts between those listed).

### Example 2—Assessments of Donor Cell Cytotoxicity Performance Separation

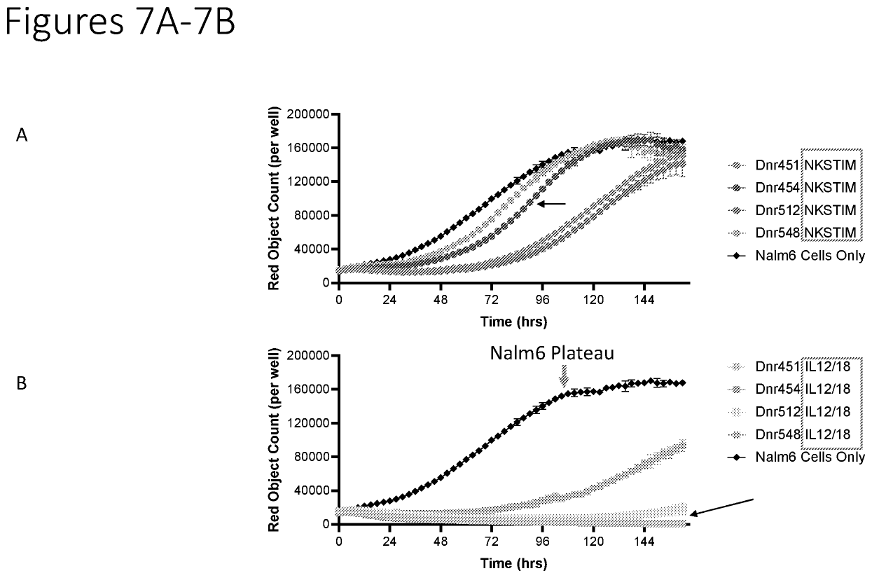

To assist in identification of donor cells exhibiting particularly desired levels of cytotoxicity studies were undertaken to determine how to separate the donors based on performance (e.g., cytotoxicity and/or expansion). These studies were also intended to help elucidate the effects of use of IL12/IL18 in the expansion process on the expanded cells. Cells were also cultured under conditions employing stimulatory molecules in the media, but utilizing a different overall expansion process (referred to in the Figures as NKSTIM, whereas the methods as provided for herein are labeled in the Figures as IL12/18). Cells were from selected donors were transduced with a non-limiting example of an anti-CD19 CAR and tested for their cytotoxicity against Nalm6 tumor cells at 14 days after completion of expansion of the cells. In FIGS. 7A-7B a 1:4 effector:target ratio was used. FIG. 7A shows the cytotoxicity profile of the cells when expanded using an alternative expansion approach and FIG. 7B shows the cytotoxicity profile of the cells when expanded using the methods disclosed herein. Overall, it is notable that the growth curves in FIG. 7B (representing tumor cell growth) are substantially muted as compared to those in 7A, indicating that the IL12/18 expansion conditions have resulted in cells that exhibit greater cytotoxicity against target cells. Nearly all donor cells using the IL12/18 expansion conditions disclosed herein nearly completely controlled tumor growth throughout the experiment and beyond the Nalm6 point of plateau. In particular, the cells from donor 454 (shown labeled with a light arrow in 7A and 7B) moved from completely controlling tumor growth in 7B to allowing almost as much tumor growth as control when NKSTIM expansion was used (7A). To further elucidate the most effective performing donor cells (in terms of cytotoxicity) another experiment was performed, but using an effector:target ratio of 1:8 (so twice as many tumor cells per engineered cell). These data are presented in FIG. 8A (NKSTIM) and 8B (IL12/18 per embodiments disclosed herein). While the greater number of tumor cells caused there to be more tumor growth in the IL12/18 expanded group, those donors still outperformed those expanded using NKSTIM. Moreover, this lower E:T ratio allowed for separation of the most effective donor cells (donor 454 and 451; see double headed arrow).

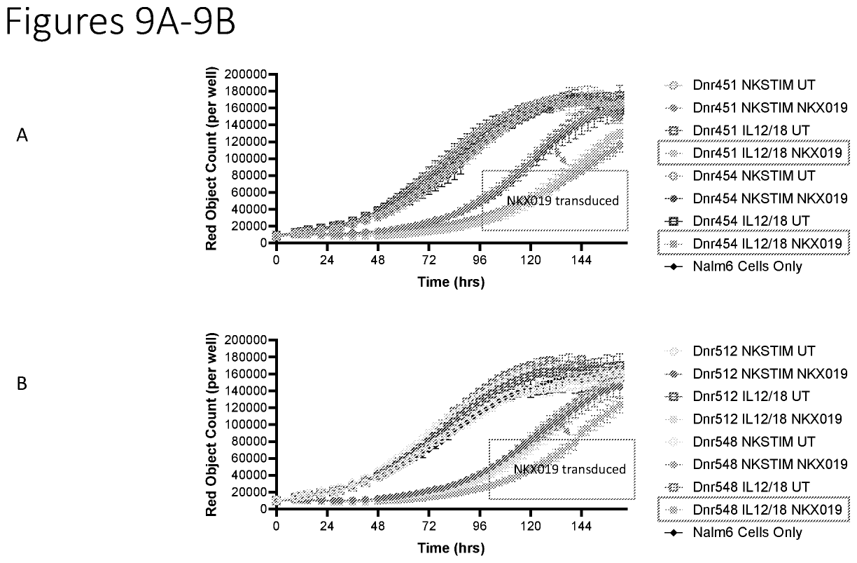

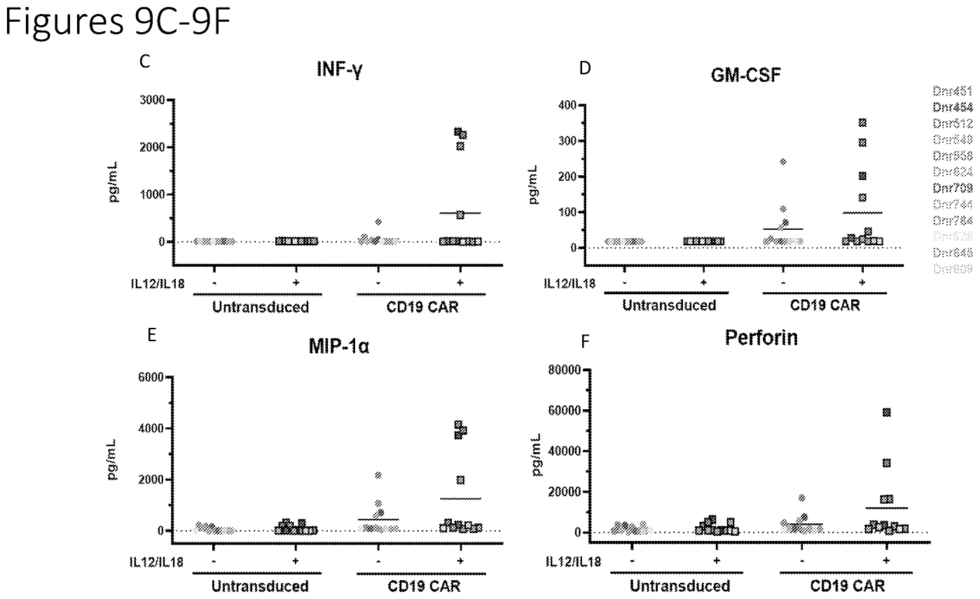

Expanded cells were evaluated for their cytotoxic persistence at longer time frames as well. FIGS. 9A and 9B show data for Nalm6 tumor challenge 21 days after completion of expansion. FIG. 9A shows data for donor 451 and 454 using either NKSTIM or IL12/18 expansion and with (NKX019) or without (“UT”-untransduced) CAR expression. Here the E:T was again 1:4. While anti-tumor activity is reduced across all donors, even at 21 days post-expansion, the use of the IL12/18 expansion conditions still drove enhanced cytotoxicity in three of the four donors tested (see boxed legend and arrows).

To further evaluate the cytotoxic effects of the engineered and expanded NK cells, supernatants were collected from a prior experiment in which engineered NK cell were tested for their cytotoxicity against NALM-6 cells, as a non-limiting embodiment of a target cell (i.e., cells as tested in NALM-6 tumor cytotoxicity assays (FIGS. 1B-1C). The supernatants were evaluated on a multi-plex panel consisting of 11 analytes. As shown in FIGS. 9C-9F, increased levels of IFN-g (9C), GM-CSF (9D), MIP-1a (9E), and Perforin (9F) were detected in IL12/IL18 expanded CD19-CAR transduced NKs. These data appeared to be consistent with the cytotoxic potency ranking of individual NK donor, see, e.g., FIGS. 2A-2C. Taken together, these data support the process of identifying a candidate donor having a desired KIR expression profile as a starting point for collecting donor cells, to be modified/expanded according to embodiments disclosed herein, in order to achieve enhanced anti-tumor effects.

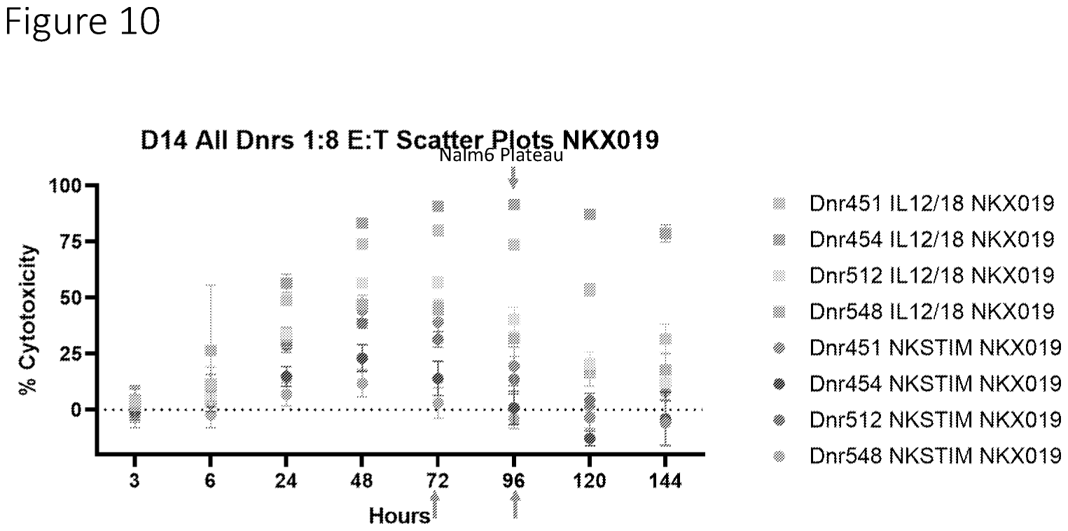

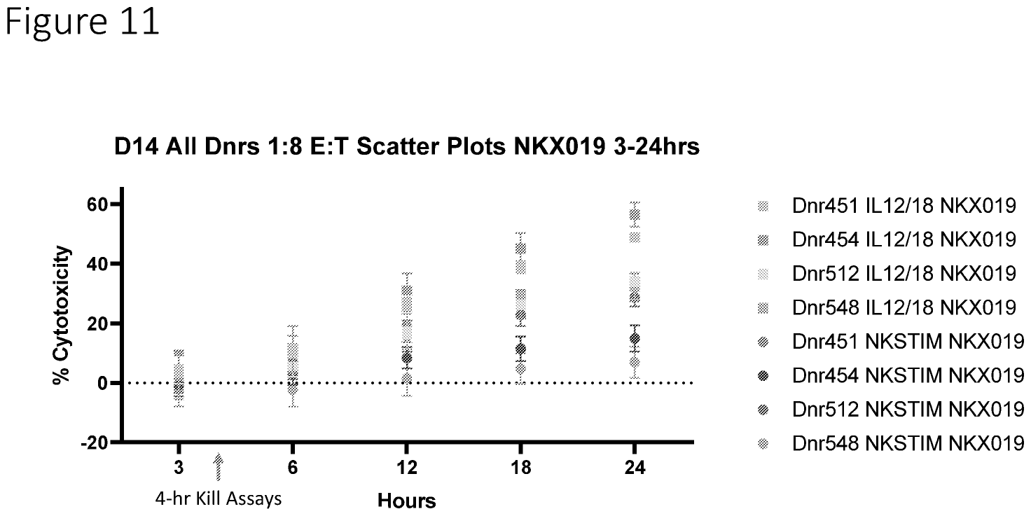

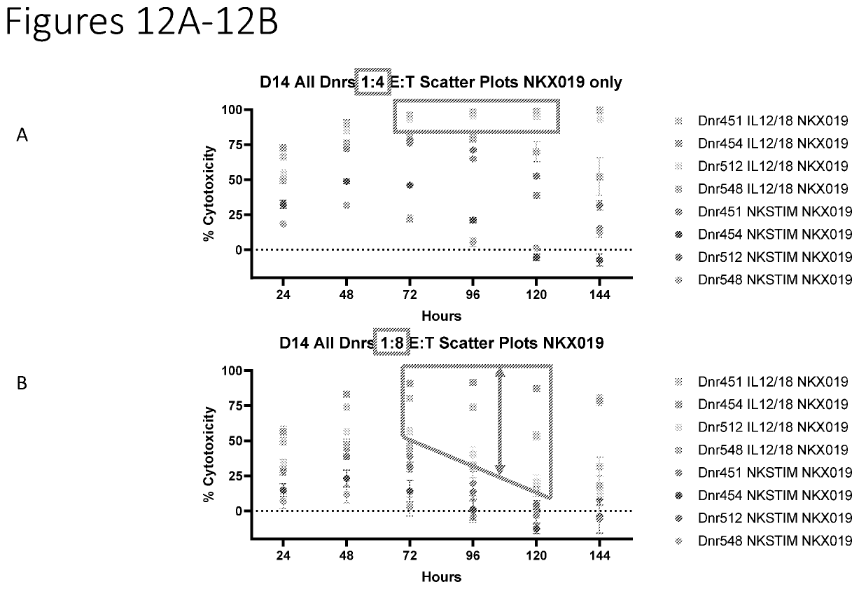

FIG. 10 shows scatter plot data of all donors tested after expansion using the IL12/18 or NKSTIM conditions and the 1:8 E:T ratio in order to help elucidate the more potent cells. FIG. 11 shows these data re-binned on earlier time points, which can help identify the more potent cells at earlier times. In several embodiments, the donor biology (e.g., KIR profile) correlates, as discussed above, with the potency as later assayed, reflecting the consideration of both donor profile and ability for expansion and responsiveness to stimulating molecules, such as IL12 and IL18, as a driver of obtaining unexpectedly effective and persistent cells for therapies. FIGS. 12A-12B show how various experimental conditions can facilitate separation of donors based on measured cytotoxicity. FIG. 12A shows cytotoxicity assessment when a 1:4 E:T ratio is used. The data curves, even when shown as scatterplots, make it challenging to separate the donors with the highest degree of cytotoxicity, due to data compression. Thus, FIG. 12B shows the same experimental setup with the exception of a 1:8 E:T ratio. As can be seen in the scatterplot, the lower E:T allows for the top performing donor cells to be separated along the Y axis (denoting % cytotoxicity), while compressing the lower performing donor cells. These data indicate that, according to embodiments disclosed herein, expansion of donor cells using multiple pulses of feeder cells and supplementation with IL12/IL18 results in enhanced cytotoxicity of the resultant expanded cells.

### Example 3—Evaluation of Expansion, Cryopreservation and Cytotoxicity

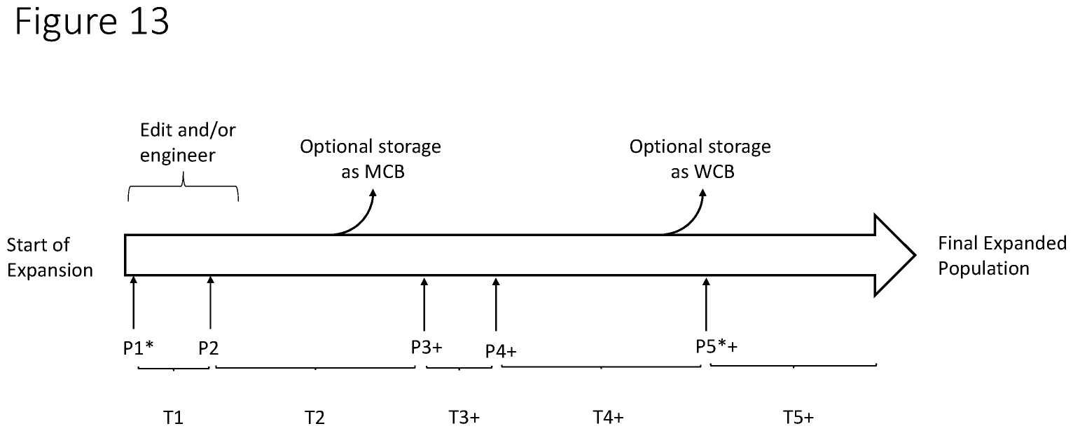

As disclosed herein, various methods for substantially expanding immune cells, such as NK cells, are provided. As discussed above, in several embodiments, these methods impart particularly effective characteristics to the resulting cells, such as cytotoxicity. Experiments were performed to examine embodiments of the expansion process and other impacts on the resulting cells. FIG. 13 shows a schematic depiction of a non-limiting embodiment of an expansion process provided for herein. As shown, the process moves from the start of expansion (using either freshly donated cells, or cells that were previously cryopreserved) to generation of a final expanded product (e.g., cells ready to be stored or administered to patients). As shown, aliquots of cells can be removed and stored as either a master cell bank (MCB) or working cell bank (WCB) for future use, for example after cryopreservation. Alternatively, the cells can be run through the process without generation of cell banks. As discussed above the cells to be expanded are co-cultured with feeder cells, as disclosed herein, with each fresh batch of feeder cells being a “pulse” or “P”. As shown in the non-limiting schematic of FIG. 13, five pulses are used in this experiment, though additional pulses could be used (as indicated by the “+” on each of the P3-P5). The time (“T”) is also indicated and can vary between the pulses, or can be consistent between one or more pulses (e.g., T1 and T3+ may optionally be the same duration). As indicated by the “*” in FIG. 13, one or more of the pulses include supplementation of the media with at least IL12 and IL18, as disclosed herein. In several embodiments, IL2 is also included. Media changes (using either IL2-free or IL2-supplemented media) are not shown and can be performed based on the visual health of the cells being expanded, the relative cell density, or other measures within ordinary skill. As shown in this non-limiting embodiment, the cells to be expanded are gene edited and/or genetically engineered early in the expansion process. In several embodiments, the gene edit occurs prior to the expansion process beginning (e.g., day-1 in the process). In several embodiments, as discussed above, donors are selected based on assessment of at least the aKIR profile of their cells. The donors used in this set of experiments were selected based on two of them (451 and 454) having aKIR/iKIR ratios that exceeded the threshold of 3. Donor 744 did not exceed that threshold.

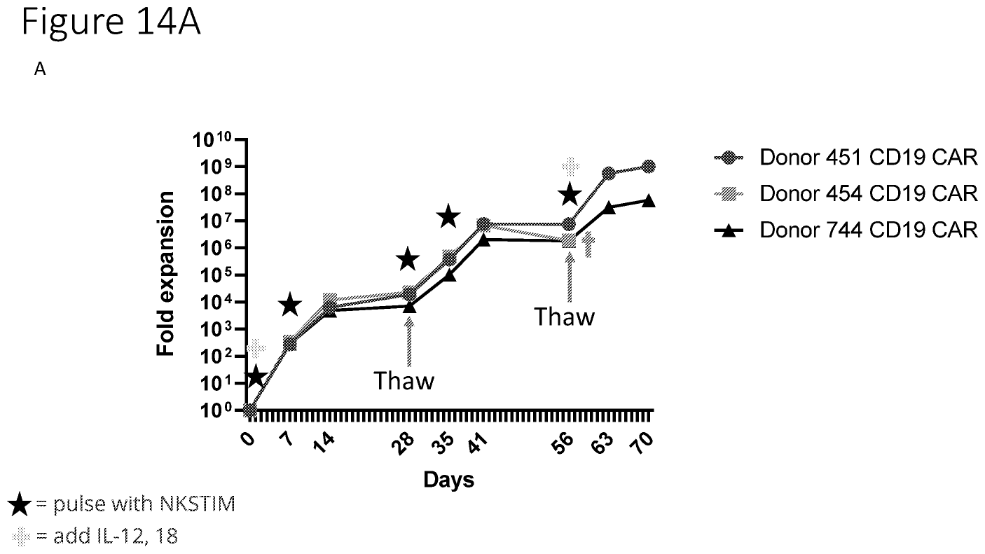

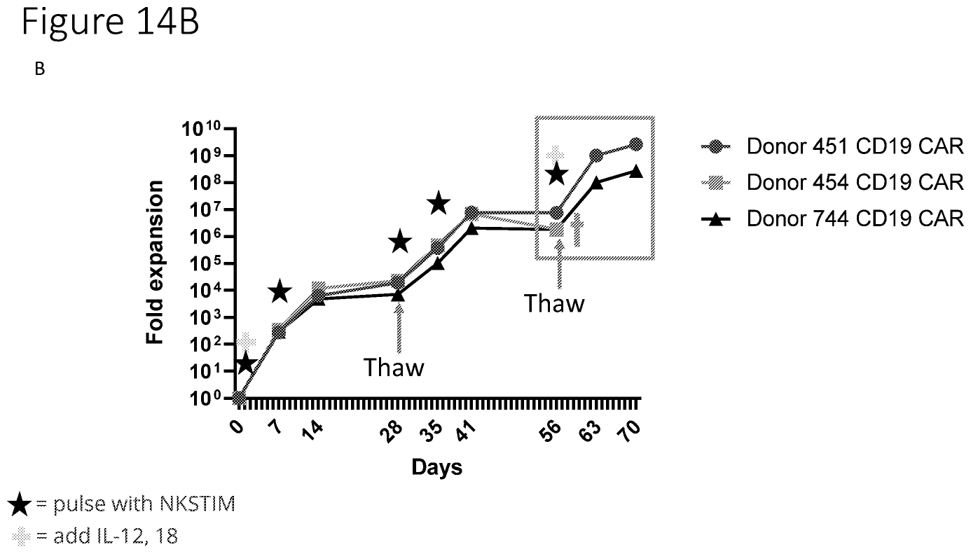

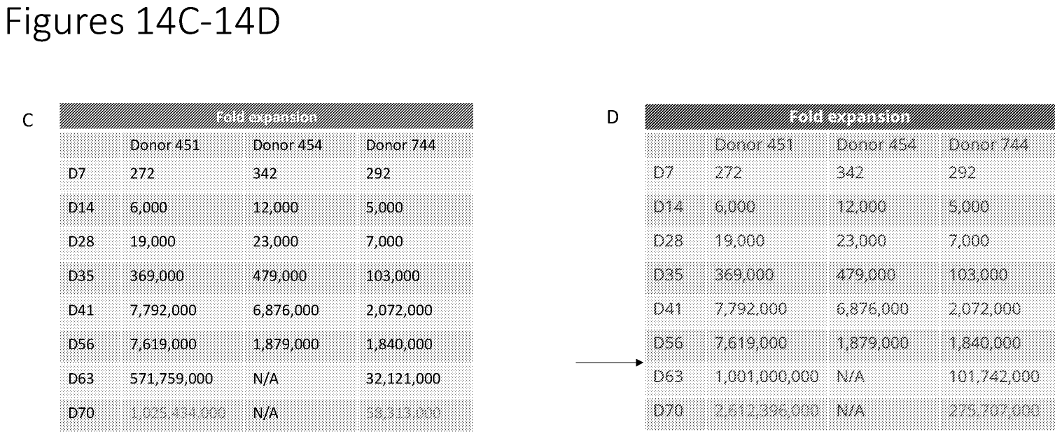

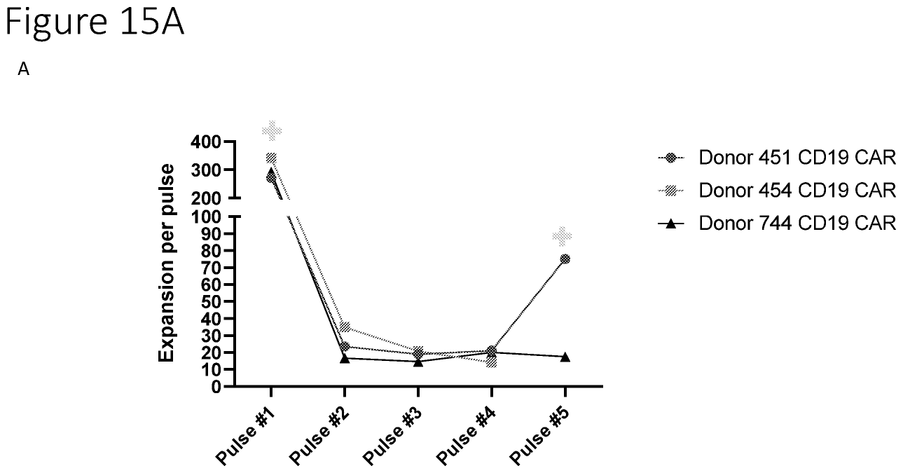

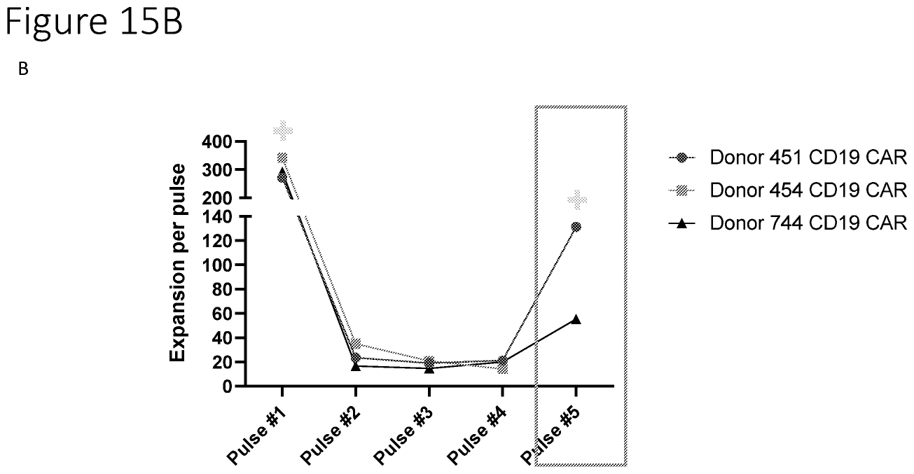

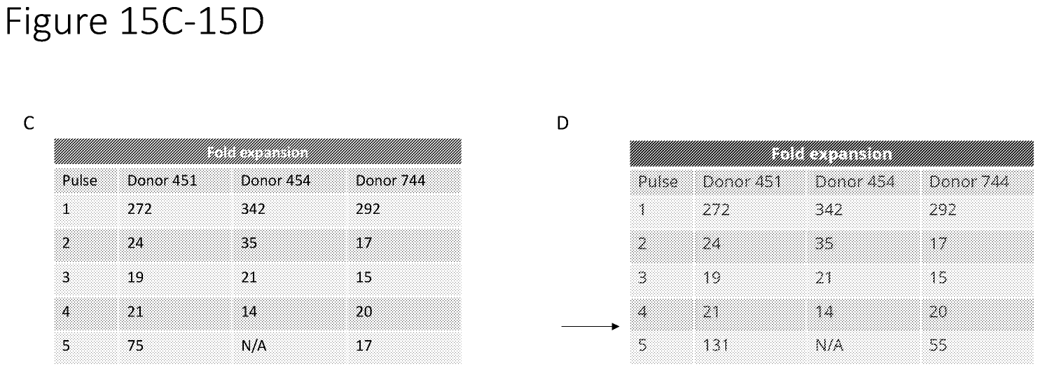

FIG. 14A shows a line graph depicting the fold expansion of cells from the three donors using a five-pulse process, as indicated. Cells were cryopreserved after pulse 2 and 4 and then thawed prior to pulse 3 and 5, respectively. IL12 and IL18 were used at pulse 1 and 5. FIG. 14B shows data from an additional experiment, where the first 4 pulses are the same data as FIG. 14A, but a new batch of cells was thawed and subjected to pulse 5 (new data is in the box). As shown, this pulsing or “multistim” process yielded unexpectedly robust expansion, with the data from FIGS. 14A and 14B being tabulated in FIGS. 14C and 14D, respectively. As shown in those tables, the least expanded cells at day 56 of the process were those from donor 454, yet even those cells had achieved nearly 2 million fold expansion (see 14C). The cells from the other two donors achieved over 1 billion-fold expansion (451) and nearly 60 million-fold expansion (744). With a repeat of the final pulse (new data is below the arrow in 14D) expansion was achieved at over 2.5 billion-fold expansion (451) and over 275 million-fold expansion (744). FIGS. 15A-15D show data as to the degree of expansion for each pulse of the process. As with FIG. 14, FIG. 15B shows the same data as for 15A for the first four pulses, with a new replicate of the experiment performed at pulse 5 (likewise for 15C and D). These data demonstrate that the process results in robust early expansion and the consistent expansion through pulses 2-4, with a possible secondary period of elevated expansion after pulse 5. These data are tabulated in FIGS. 15C and 15D, which show expansion exceeding 270-fold at pulse 1, ranging from about 15 to 30 fold at pulses 2-4, and returning to elevated levels at pulse 5. These data show that, as according to several embodiments, the use of IL12 and IL18 at pulse 5 causes the cells to enter an additional period of expansion, which results in a substantial number of cells that are available for clinical use (optionally preceded by cryopreservation).

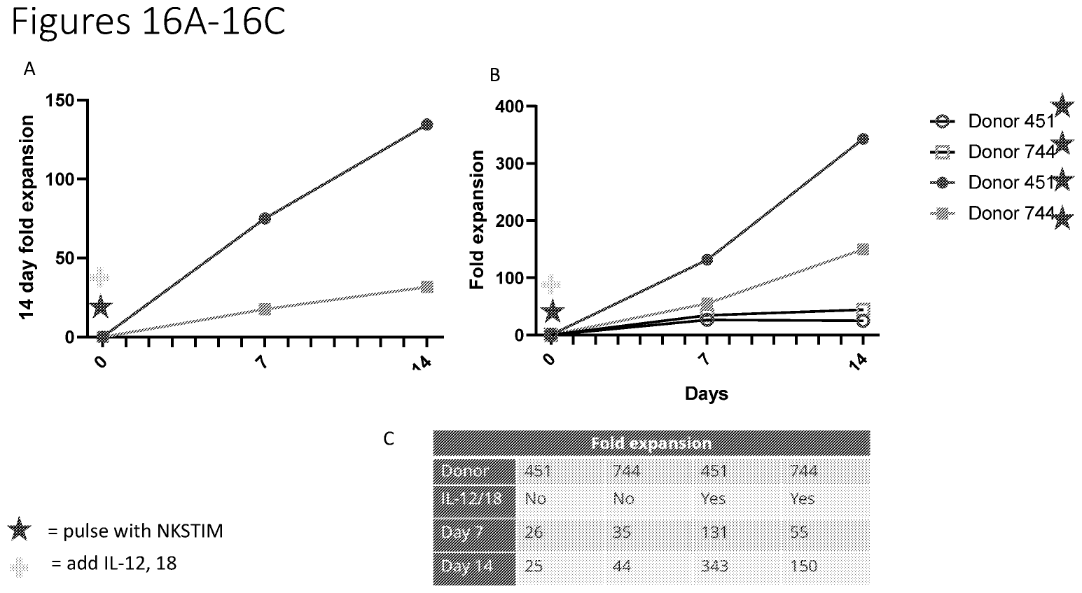

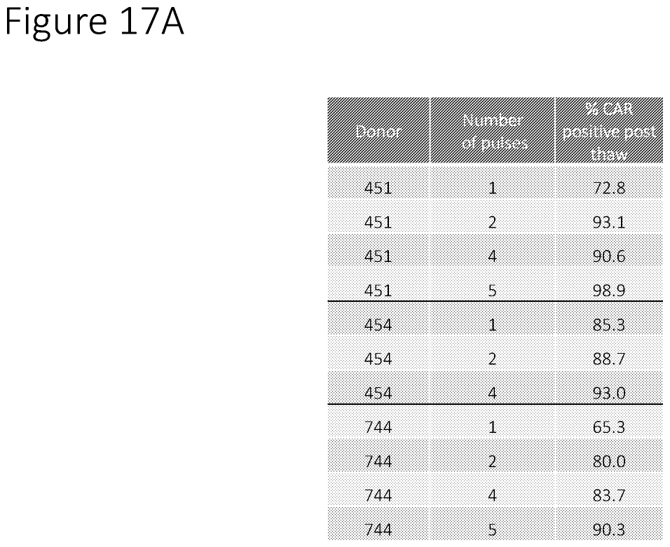

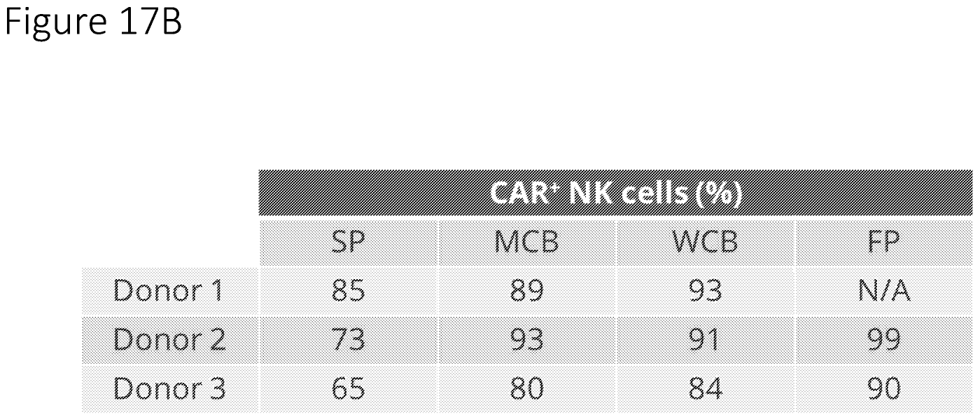

Further experiments were conducted to elucidate the impact of the IL12 and IL18 added at pulse 5. FIG. 16A shows expansion data for the final 14 days of culture (from pulse 5 to final product). FIG. 16B shows similar data from another replicate in which IL12 and IL18 were either present (solid) or absent (open). FIG. 16C tabulates this data which shows that the presence of IL12 and IL18 marked enhances the expansion of the cells at the final pulse. As the expanded cells are transduced to express a CAR early in the expansion process, FIG. 17A shows that, importantly, over 90% of the cells still express the CAR at the close of expansion. It should be noted that these cells were also genetically edited to reduce expression of CISH, according to embodiments disclosed herein. This advantageously results in a highly expanded cell population that is enriched with respect to CAR positivity. FIG. 17B breaks down similar expansion data based on the stage of production according to methods disclosed herein. SP refers to expansion of NK cells using K562 cells modified to express mbIL15 and 4-1BBL as feeder cells and including soluble IL12 and IL18 in the culture media (termed “NKSTIM”; see, for example International Patent Application No. PCT/US2020/044033, filed Jul. 29, 2020, the entire contents of which is incorporated by reference herein). MCB refers to Master Cell Bank, WCB refers to Working Cell Bank, and FP refers to Final Product (see e.g., FIG. 13). These data show that at each stage of the expansion process (other than Donor 1 at FP), significant percentages of the cells retain expression of the transduced CAR, meaning that even after significant expansion, the expression of the CAR is persistent, and a substantial portion of the post-expansion cells remain therapeutically relevant.

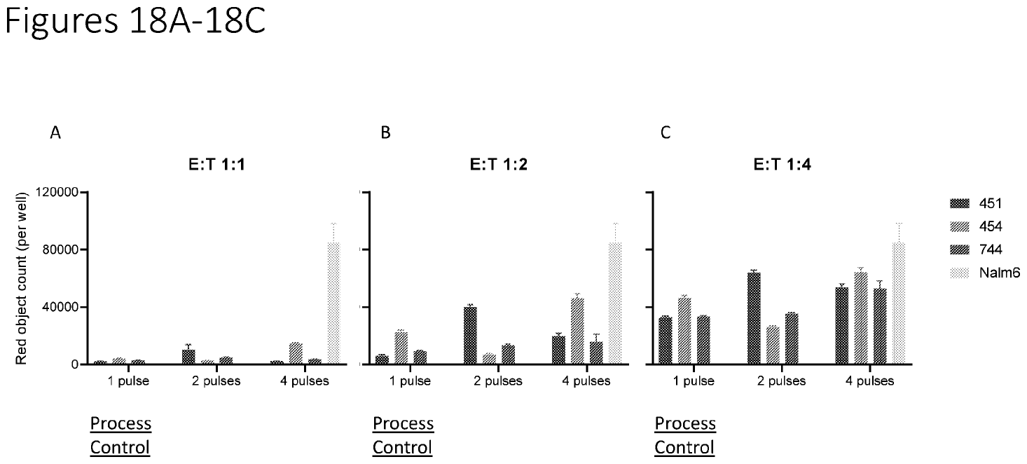

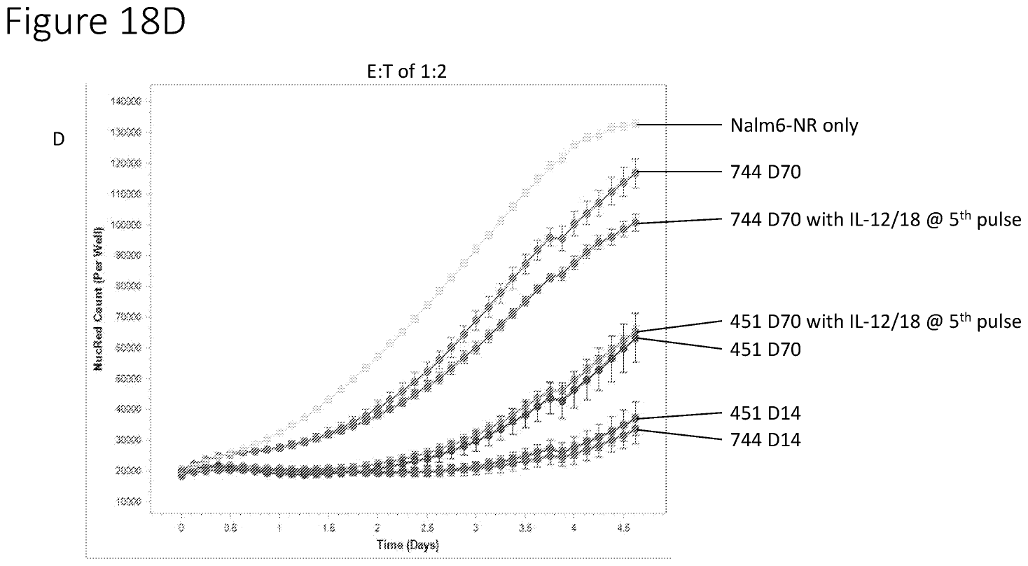

Moreover, the cell population post-expansion has been demonstrated to be functional (e.g., cytotoxic) during, and after, expansion. FIGS. 18A-18C show summary data related to the cytotoxicity of the cells at either 1:1 (18A), 1:2 (18B) or 1:4 (18C) E:T ratios with assays being performed at 1, 2, and 4 pulses (pulse 1 being a control expansion process using feeder cells and IL12/IL18. Red object count (tumor cell) is substantially lower than control (Nalm6 alone) at all pulses across all E:T, indicative of potent cells. FIG. 18D shows tumor growth curves when cells at the completion of the expansion process were co-cultured with the Nalm6 tumor cells when IL12/18 were included, or not, at the final expansion pulse. These data show that, while Day 14 process control cells effectively control tumor growth, the cells expanded for the full process also demonstrate a favorable profile, particularly for donor 451 (aKIR/iKIR ratio above threshold). Donor 744 (aKIR/iKIR below threshold) allowed Nalm6 growth towards the end of the experiment, but also shows the positive impact of IL12/IL18 on cytotoxicity (the lower curve for donor 744). Thus, according to embodiments disclosed herein, immune cells, such as NK cells, that are expanded according to embodiment disclosed herein retain a high degree of cytotoxicity. Coupled with the much larger number of cells, this allows for multiple patient doses to be generated and stored, thereby facilitating effective allogeneic cell therapy.

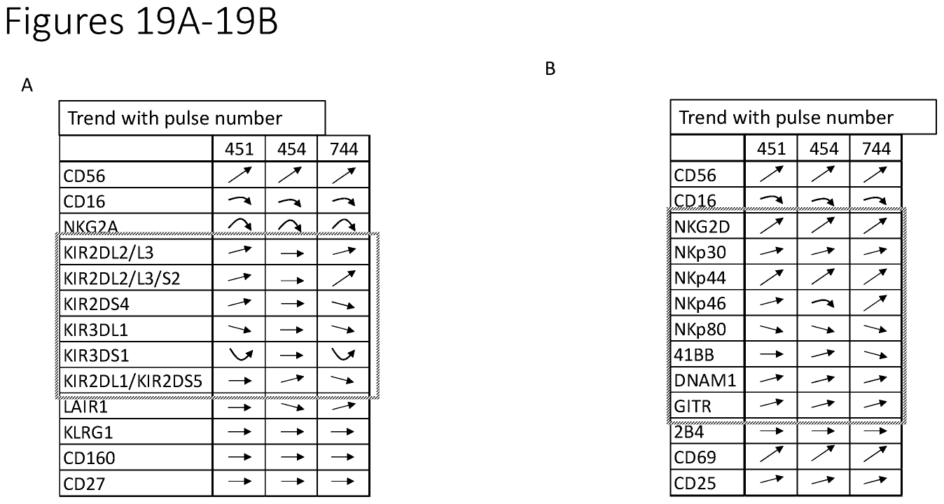

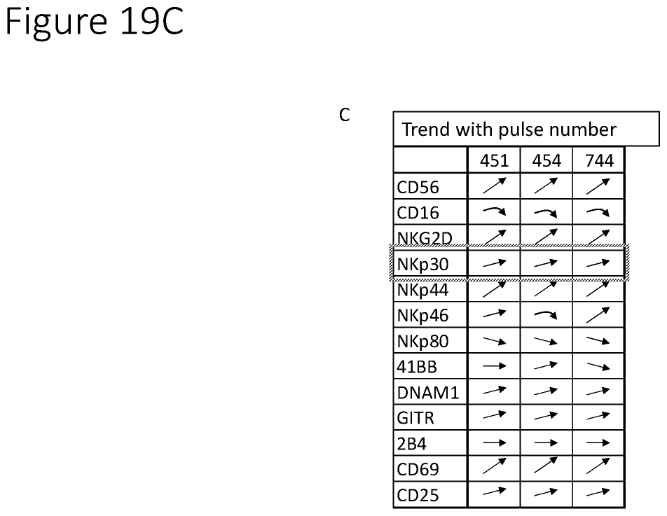

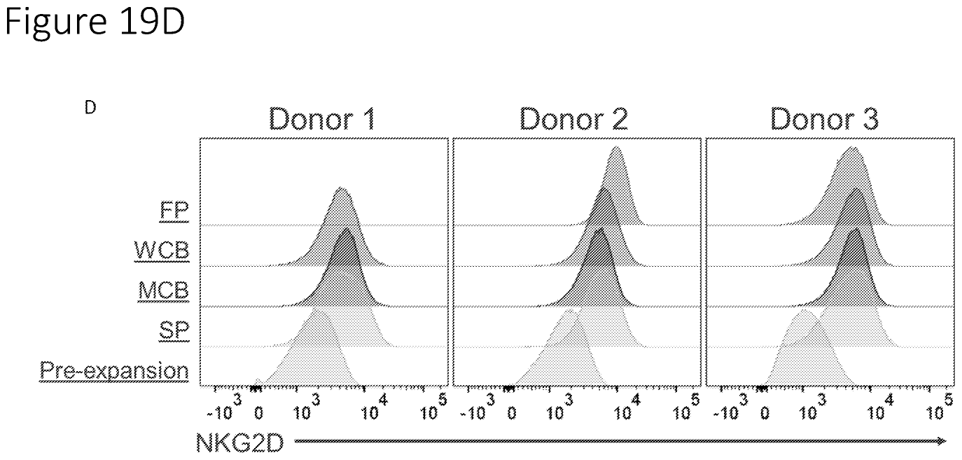

Looking further into the potential mechanism of action underlying the cytotoxicity imparted to the expanded cells, various phenotypic assays were performed on the cells during the expansion process. FIG. 19A shows the general trend of increased KIR expression with increasing pulse number. Both aKIR and iKIR expression seemed to trend upwards. FIG. 19B shows data that indicates that several activating receptors increase in expression with pulsing during expansion. Notable among these is the expression of NKp30 (FIG. 19C) which shows the trend for increased expression over the initial four pulses of the expansion. NKp30 is one of the natural cytotoxicity receptors, a family of immunoglobulin (lg)-like NK cell activation receptors, that has been shown on human NK cells to be key receptors in tumor immunity. These data indicate that many activating receptors undergo upregulated expression during expansion. FIG. 19D summarizes, for three donors, the expression of the NKG2D surface expression on NK cells. As can be seen for each donor, as compared to a pre-expansion control (bottom row), expansion of NK cells with the Standard Procedure (“NKSTIM” as discussed above, see for example International Patent Application No. PCT/US2020/044033, filed Jul. 29, 2020, the entire contents of which is incorporated by reference herein) results in greater expression of NKG2D (right shift of the curve). Similar trends were observed for NKp30, NKp44, NKG2C, DNAM-1, KIR2DS4, and KIR3DS1 in most donor NK cells. During the progression through the multi-phase expansion processes embodied herein (Master Cell Bank to Working Cell Bank to Final product), the expression of NKG2D generally continues to increase (right shift of curve moving up from MCB to WCB to FP). As discussed in more detail below, the continued increase in activating receptors is believed to engender the cells with long lasting cytotoxic potency against target tumor cells, even after significant (and for some donors pre-terminal expansion limits) expansion. As a result, in several embodiments, the expansion methods disclosed herein result in a sizeable, cytotoxically potent, and persistence population of cells for cancer immunotherapy.

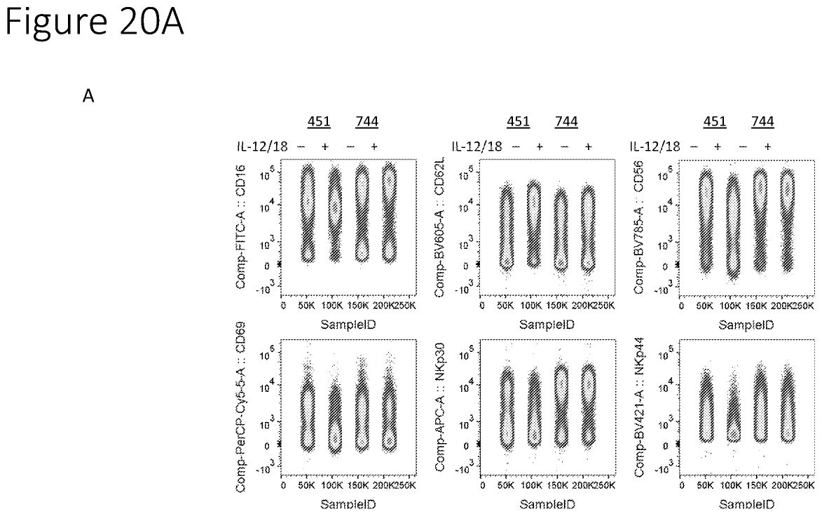

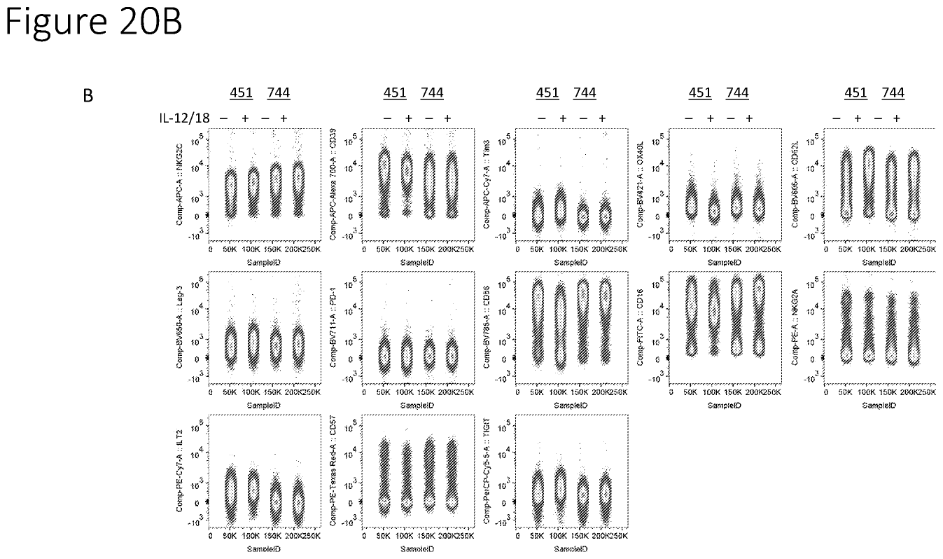

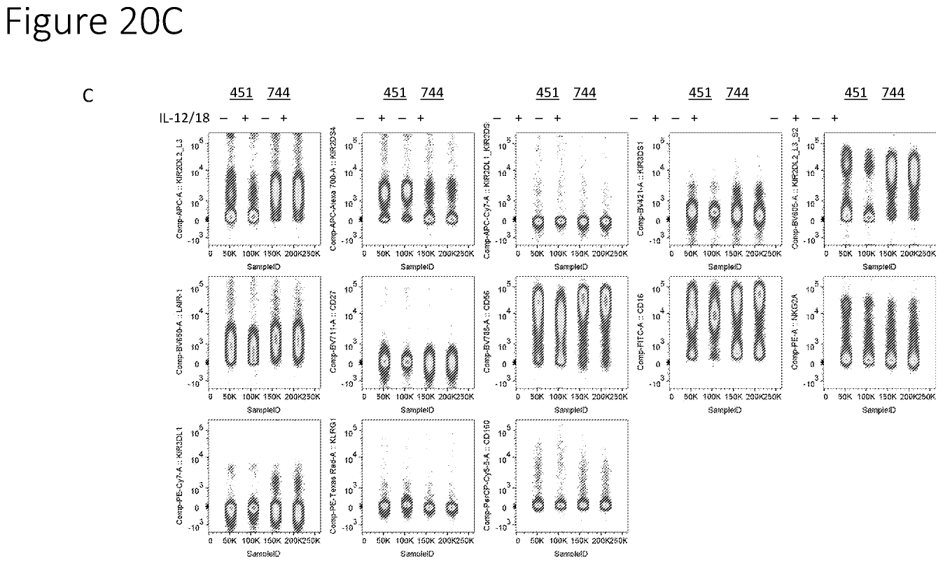

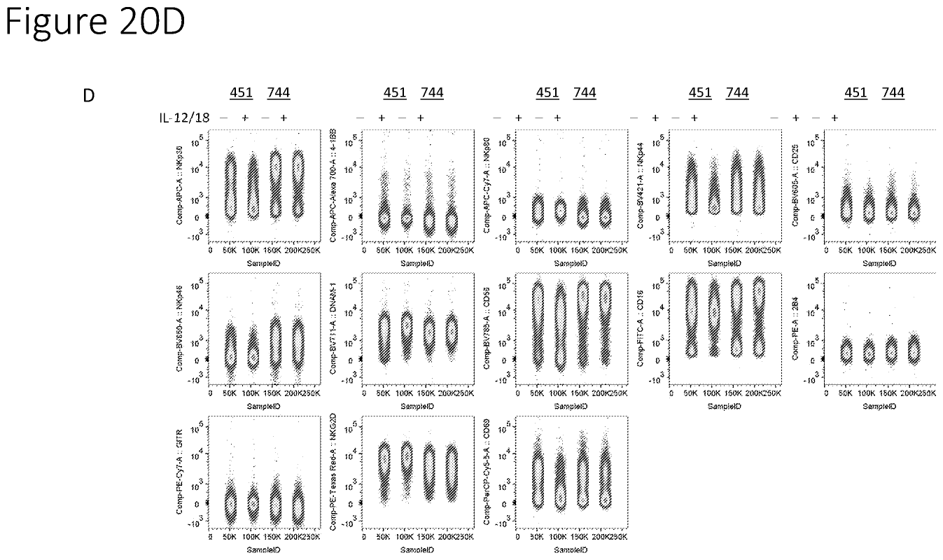

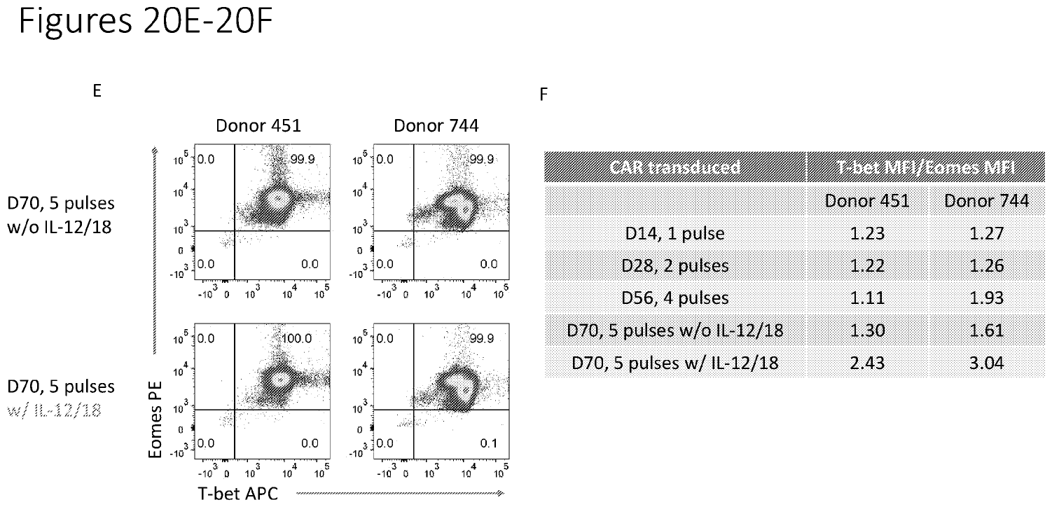

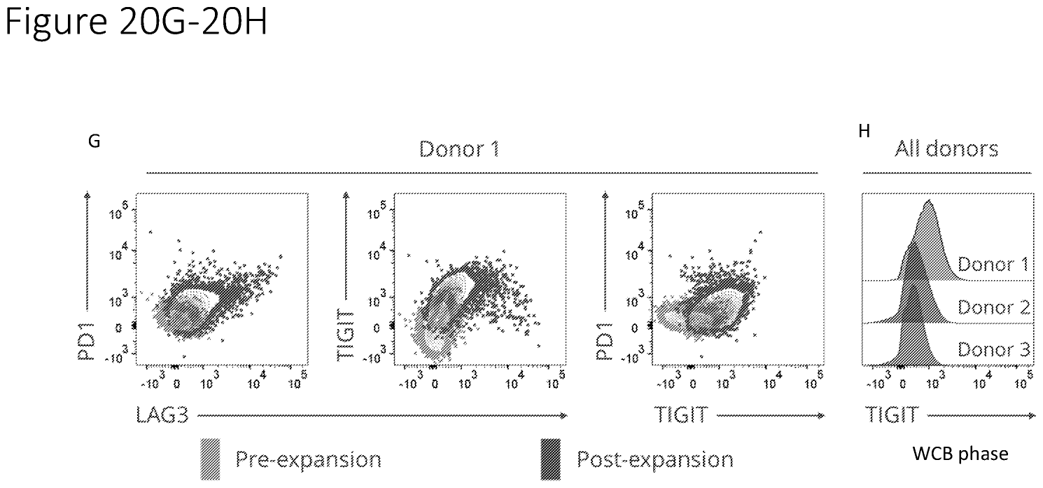

FIGS. 20A-20F show expression of various markers on the NK cells of two donors when expanded with or without IL12/IL18 at the final pulse. There are several notable differences (see FIG. 20A, which is a selection of data from FIGS. 20B-20C) in expression when IL12/IL18 is present, such as the increased expression of CD62 ligand (CD62L), which, at least on T cells, functions as an activation marker, as opposed to a memory marker. Memory T cells are known to be less responsive to tumor cells as compared to naive T cells, thus the IL12/IL18 induced increase in CD62L may be resulting in a population of more active NK cells. FIGS. 20E and 20F show the impact of Il12/IL18 on expression of T-bet and Eomes, two T-box transcription factors that regulate NK cell development and activity. The elevated expression of these two transcription factors may be involved in the enhanced cytotoxicity exhibited by cell expanded using Il12/IL18 at the final pulse. FIGS. 20G-20H show data related to the expression of markers of exhaustion on NK cells. FIG. 20G shows plots of various markers for Donor 1, who was the donor whose cells did not expand to the FP phase (as compared to Donors 2 and 3, which expanded over 2 billion and 200-million fold respectively), though they did expand ˜7 million-fold before undergoing contraction. PD-1, LAG3 and TIGIT are established markers of exhaustion in T cells, and the experiment discussed here was to determine how the expression of these markers changed on NK cells during expansion, in particular for Donor 1, whose expansion lagged behind that of Donor 2 and Donor 3. In the left panel of FIG. 20G, there is an increase in both PD-1 and LAG3 (comparing pre-expansion to post-expansion), with a slightly greater LAG3 increase (evidenced by the shift of the plot up but also slightly more to the right). The central panel of FIG. 20G shows a fairly dramatic increase in TIGIT expression as compared to LAG3 (vertical shift of plot) and likewise, the right panel of 20G show a significant right shift (TIGIT) as compared to vertical upshift of PD-1. These data suggest that TIGIT expression increasing based on NK cell expansion may drive an exhaustion-like phenotype that could be responsible for the limited expansion of Donor 1. FIG. 20G shows TIGIT expression at the WCB phase for all three donors, and Donor 1 notably exhibits a greater expression of TIGIT at that stage of expansion. Thus, in several embodiments, cells may be optionally evaluated during expansion (e.g., at the WCB phase, or some other time prior to FP generation) for TIGIT expression levels. In several embodiments, an elevated TIGIT expression level can result in termination of the expansion of those cells, on the premise that the overall expansion of those cells will not reach the full potential of the methods disclosed herein (e.g., for a donor expressing lower TIGIT levels).

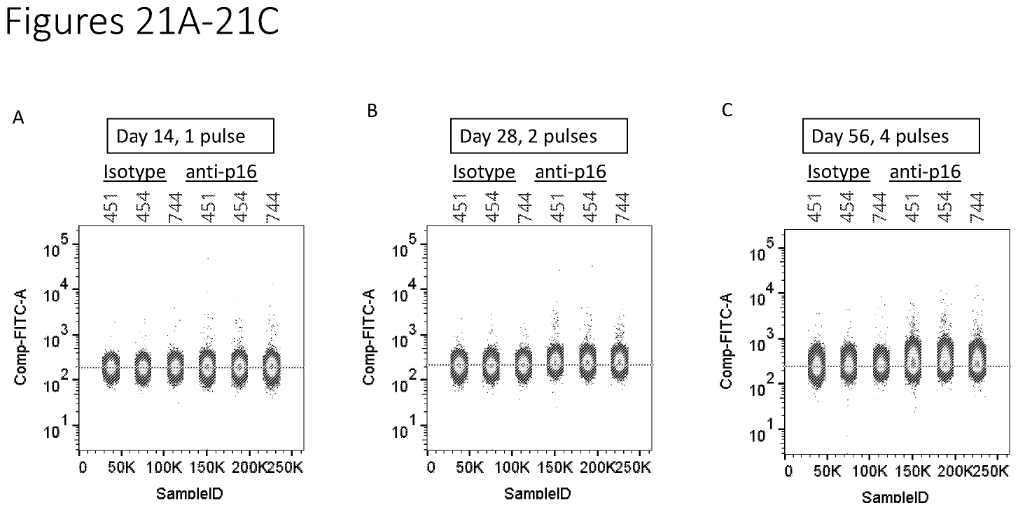

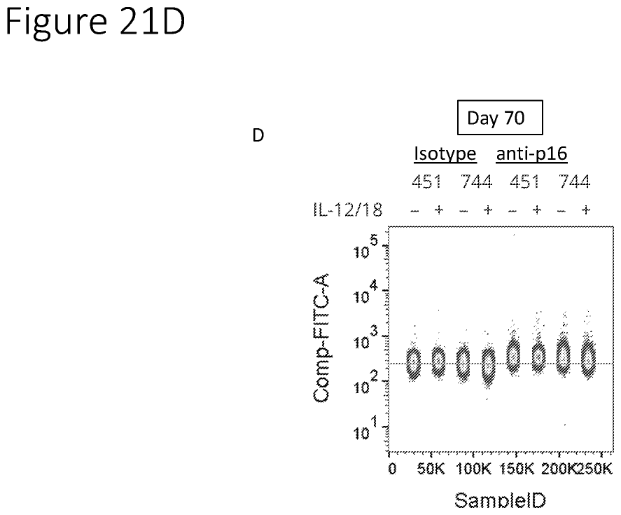

FIGS. 21A-21D trace expression p16INK4a (“p16”), which is known as a marker of aging in certain immune cells, particularly T cells and NK cells. Expression of p16 was relatively constant in the expanded NK cells at day 14, 28 and 56 of expansion, whether or not IL12/IL18 were present in the media. A subtle decrease in p16 expression was detected at day 70 of expansion with the inclusion of IL12/IL18 in the media at the pulse prior to this timepoint. This indicates that, according to several embodiments, the cells pulsed with IL12/IL18 at the final pulse of expansion do not appear to have reached a terminal expansion limit (such a limit would be associated with high p16 expression) as is seen when expanding cells that have reached senescence.

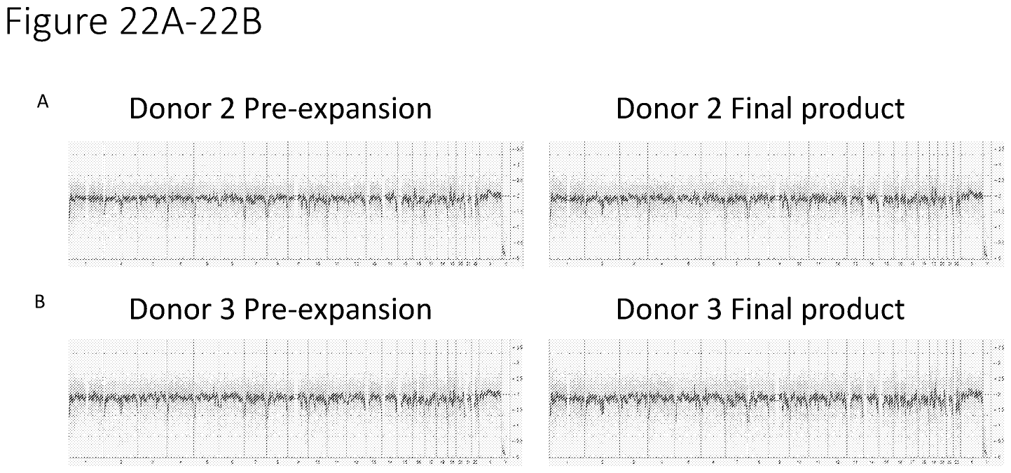

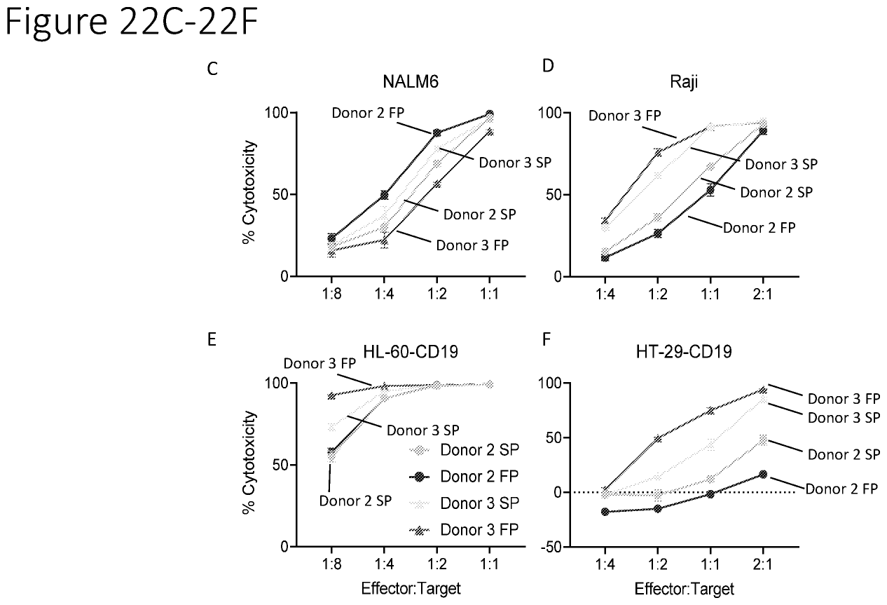

FIGS. 22A-22F show further data around the cells expanded from Donor 2 and 3. With such significant levels of expansion, there is concern that genetic abnormalities could be generated (e.g., aging cells have reduced telomeric length and therefore are subject to potential mutation and/or less effective DNA repair mechanisms (not because they are less effective on a division to division basis, but because there are so many divisions with this degree of expansion)). FIGS. 22A and 22B show chromosomal analysis across 150 single nucleotide polymorphisms from Donor 2 pre- and post-expansion (22A) and Donor 3 pre- and post-expansion (22B). The X-axis is the chromosome number and Y-axis indicates copy number. As can be seen, there is relatively consistent copy number for both donors at each of the SNPs evaluated, indicating that the cells from these Donors are genetically stable over time, despite the significant expansion. Thus, in several embodiments, donor cells exhibiting genetic stability reduced the risk of, for example, the expanding/expanded cells becoming cancerous themselves.

FIGS. 22C-22F show data related to the maintained cytotoxicity of extensively expanded cells from Donors 2 and 3 against multiple tumor cell types. Standard Process (SP) and Final Product (FP, according to methods disclosed herein) NK cells from Donor 2 and Donor 3 (and engineered to express an anti-CD19 CAR and mbIL15) were used in a cytotoxicity assay against B cell tumor cell lines that naturally express CD19 (NALM6 and Raji) and non-B cell tumor cell lines that ectopically express CD19 (HL-60-CD19 and HT-29-CD19). Percent cytotoxicity was calculated based on Incucyte images collected at 72-hours after co-culture. % cytotoxicity= [(control-experiment control]×100. As can be seen in each panel, depending on E:T ratio, extensively expanded NK cells exhibit cytotoxic effects against both tumor cells naturally expressing CD19 and those expressing CD19 ectopically. A notable trend in the data is a fairly close correlation between the cytotoxicity of the SP and FP cells within a given donor, indicating that while the FP cells have been far more extensively expanded, this greater cell number does not come at the expense of cytotoxicity. Thus, according to some embodiments, the methods disclosed herein generate NK cells that are about, if not more, potent than SP-expanded NK cells, and those cells are generated in a significantly greater quantity, shifting the manufacture of NK cells in an off-the-shelf allogeneic format from a future desirable goal, to an accomplished manufacturing process.

### Example 4—Evaluation of Expansion and Cytotoxicity of NK Cells from Cord Blood and Peripheral Blood

In order to further investigate the capacity for expansion of immune cells, such as NK cells, further experiments were performed to compare immune cells derived from cord blood (CB) samples or peripheral blood (PB) samples. Cells were expanded according to the methods disclosed herein, which are briefly summarized below (see also, FIG. 13). A first period of expansion is performed, which comprises between 25-35 days of expansion (e.g., ˜28 days) and comprises two co-culturings (e.g., “pulses”) of cells being expanded with feeder cells. As discussed herein, the feeder cells, according to several embodiments, comprise cells that express mbIL15, 41BBL and are optionally low-expressing or devoid of MHCI (such as K562 cells). Soluble IL12 and/or soluble IL18 are used to supplement the culture media for at least one of the co-culturings in this first period. Cells are optionally frozen after this first period. Additionally, as performed here, cells can be characterized (e.g., phenotype or cytotoxicity evaluated, among other features), which can optionally serve as a gating event for the remaining expansions (e.g., if cells do not demonstrate desired characteristics after this phase, they need not be further expanded). A second expansion phase comprises between 25-35 days of expansion (e.g., ˜28 days) and comprises two co-culturings (e.g., “pulses”) of cells being expanded with feeder cells. As after the first expansion, cells can optionally be evaluated at this stage as well. A third expansion phase is performed, which comprises 12-15 days (e.g., ˜14 days) and comprises a single “pulse” with the feeder cells as well as media supplementation with soluble IL12 and/or IL18. In total, the expansion process comprises, in several embodiments, three phases that span ˜70 days. IL2 is optionally used at one or more of the co-culturings, with concentrations ranging from about 40 to about 500 U/mL. As disclosed herein, cells may also be genetically edited (e.g., to reduce or knockout expression of a target gene/protein) and/or engineered to express, for example a CAR targeting a tumor marker of interest.

NK cells were obtained from peripheral blood of a donor and from three individual cord blood samples. Each set of cells was expanded according to embodiments disclosed herein. The PB NK and CB NK cells were co-cultured (at Day 0) with K562-mbIL15-41BBL feeder cells (at a 1:3 NK:feeder cell ratio) in growth media supplemented with IL12, IL18, and IL2. In several embodiments, the IL18 is present in the supplemented media between about 10 ng/ml and about 30 ng/ml and the IL12 is present in the supplemented media between about 0.01 ng/ml and about 10 ng/mL. In several embodiments, the media is supplemented with between about 25 and about 50 U/mL of IL2. After ˜4 days, the media was supplemented again with IL2 at an elevated concentration. In several embodiments, the elevated concentration ranges from about 300 to about 500 U/mL. At Day 5, both PB NK and CB NK cells were transduced with a non-limiting embodiment of a CD19-directed CAR and mbIL15 (or mock transduced). Transduction was at a multiplicity of infection of 1.5, as a non-limiting embodiment. The media was again supplemented with an elevated concentration of IL2. The expanding PB NK or CB NK cells were pulsed again fresh feeder cells (at Day 7) and using media supplemented with IL2 at the lower concentration. The PB NK and CB NK cells were co-cultured for another 14 days before being assayed and/or cryopreserved. However, in several embodiments, the cells need not be cryopreserved, but can proceed directly to the next phase of expansion.

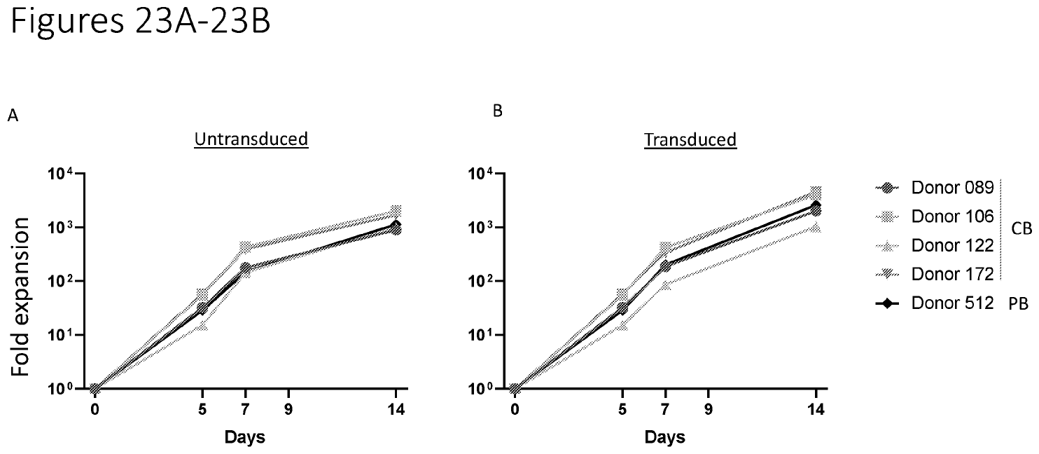

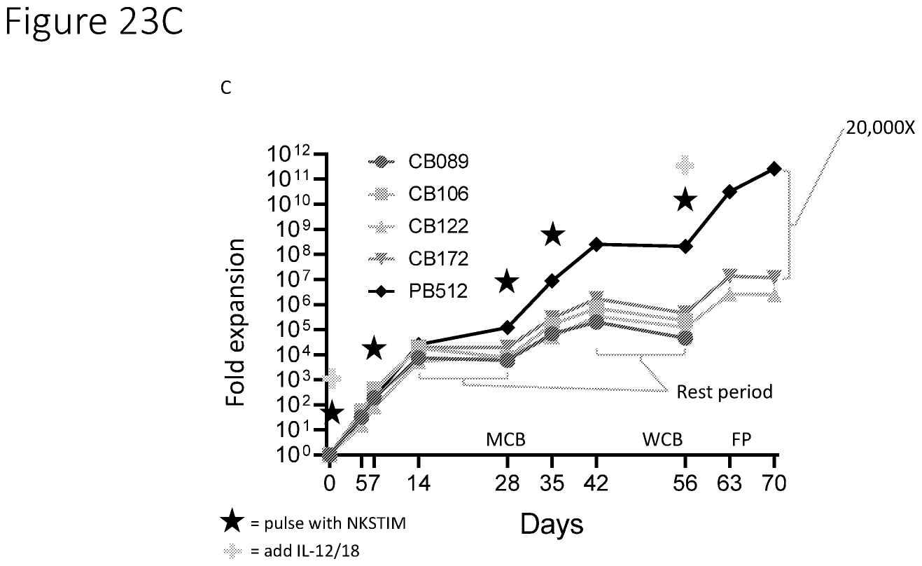

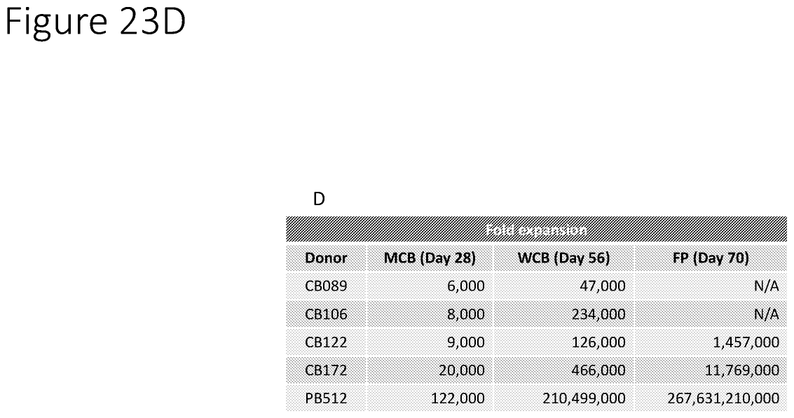

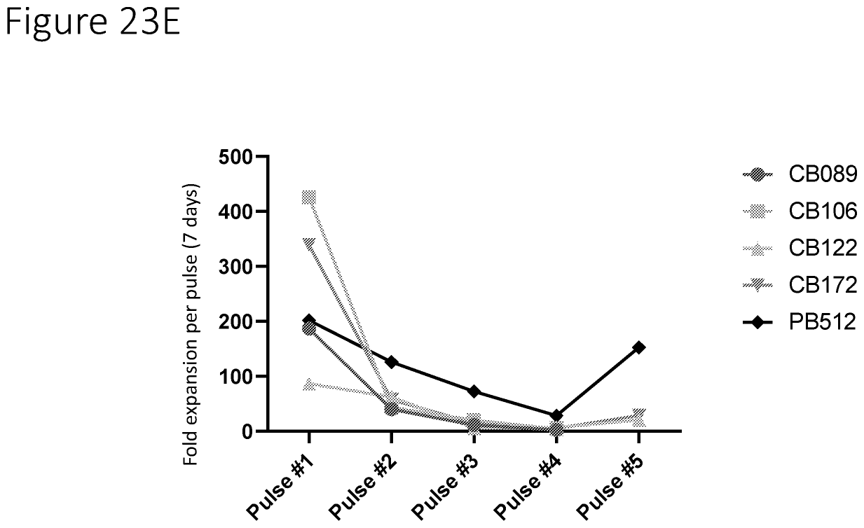

FIGS. 23A and 23B show data collected after the first expansion. FIG. 23A shows the fold expansion of mock transduced PB NK or CB NK cells. These data show that NK cells from either source are responsive to the expansion process over the first 14 days, with neither cell type showing a clearly enhanced expansion potential. FIG. 23B shows corresponding data from those cells transduced with the CD19-directed CAR. These data show, in accordance with several embodiments, that the expression of a tumor directed CAR does not appear to significantly dampen the expansion potential of NK cells either from cord blood or from peripheral blood.

Moving to expansion phase 2, frozen cells were thawed and subjected to an additional co-culture (third pulse, now at Day 28 of the process or Day 0 of phase 2). At this stage the expanded PB NK or CB NK cells were cultured with fresh feeder cells in media supplemented with low concentration of IL2. Seven days later (Day 35 overall, Day 7 of phase 2) the PB NK and CB NK cells were pulsed again with fresh feeder cells (pulse number 4). The PB NK and CB NK cells were cultured for approximately 21 days (through Day 56 overall, Day 28 of phase 2). The cells were cryopreserved at the end of that co-culturing (with a set of cells separated for phenotyping). However, in several embodiments, the cells need not be cryopreserved, but can proceed directly to the next phase of expansion.

The final expansion phase (phase 3) involves thawing the cells from the prior phase (or directly proceeding with cells that were not cryopreserved). The PB NK or CB NK cells were co-cultured with the feeder cells (pulse #5) using IL12/IL18 supplemented culture media, which was also supplemented with the lower concentration of IL2. The NK cells were co-cultured for approximately 14 days (totaling Day 70 in the overall process, Day 14 of phase 3). A portion of the cells was separated for phenotyping, while the remainder were cryopreserved. According to some embodiments, the cryopreserved cells are frozen in suitable for thawing and administration to a patient. In some embodiments, a subset of cells may be administered to a patient without being cryopreserved.

FIG. 23C shows data tracing the expansion of PB NK and CB NK cells across the three phases of expansion (5 pulses of feeder cells). Each pulse is indicated with a “star” symbol and each supplementation of the media with IL12/IL18 is indicated with a “plus” symbol. The phases are indicated above the X axis (MCB=Master Cell Bank, or phase 1; WCB=Working Cell Bank, or phase 2; FP=Final Product, or phase 3). The expansion curves for each of the sets of cells (four CB samples, and one PB sample) show that robust expansion of all cell samples occurred during phase 1. While all CB NK cells experienced modest expansion across phase 2, reaching between about 105 and about 10⁶-fold expansion, two of the CB NK cell samples continued to expand through phase 3 reaching a final expansion in this experiment of about 10⁶-10⁷-fold. PB NK cells showed a similar expansion as CB NK cells through the initial portion of phase 1, but by the end of phase 1, PB NK cells appeared to expand to a greater degree, reaching about 10⁵-fold expansion. Thereafter, the PB NK cells showed greater expansion throughout the remainder of the expansion process, with the final expanded cell population being approximately 20,000 times that of the CB NK cells. FIG. 23D tabulates the expansion data of FIG. 23C. FIG. 23E shows a breakdown of the expansion on a “per-pulse” basis. These data reflect that the initial pulse provides significant expansion for all cells (regardless of cell type), but the later phases appear to induce lesser degrees of expansion in CB NK cells, as compared to PB NK cells, which had an additional significant expansion at the fifth pulse, reaching nearly the same degree of expansion as pulse 1. Taken together, these data show that cell expansion methods as provided for herein result in substantial expansion of cell populations. In several embodiments, this allows for production of immune cell populations in quantities that are clinically relevant for a plurality of patients. These data also show that cells engineered to express a CAR, and optionally that are gene edited to reduce expression of one or more target genes/proteins, are amenable to expansion. These data also suggest that, in several embodiments, use of a peripheral blood sample is preferred as starting materials for NK cell expansion, though, as shown, cord blood samples still yielded significant expansion.

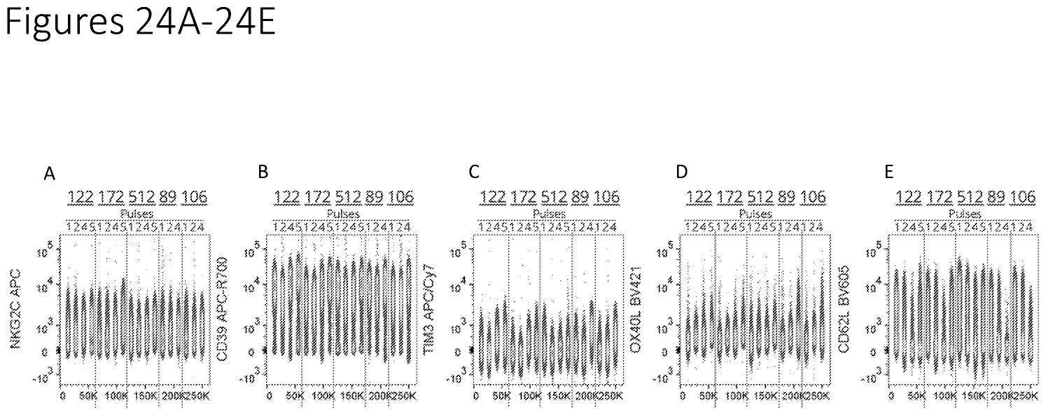

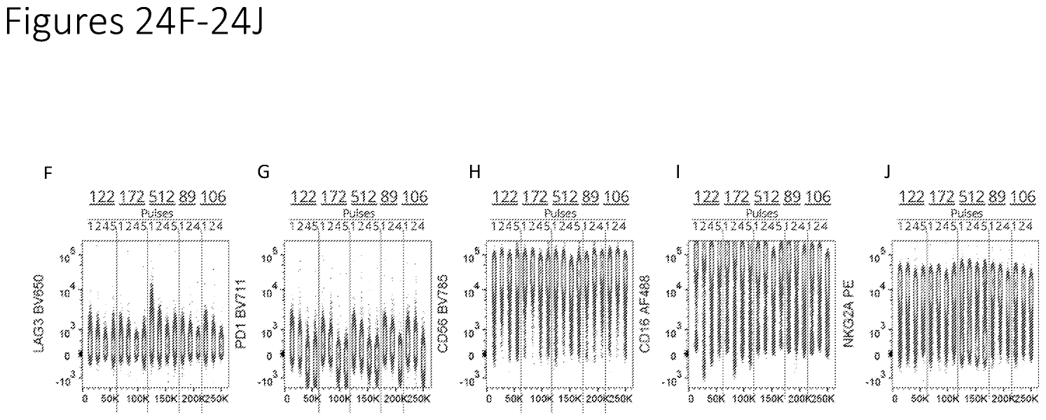

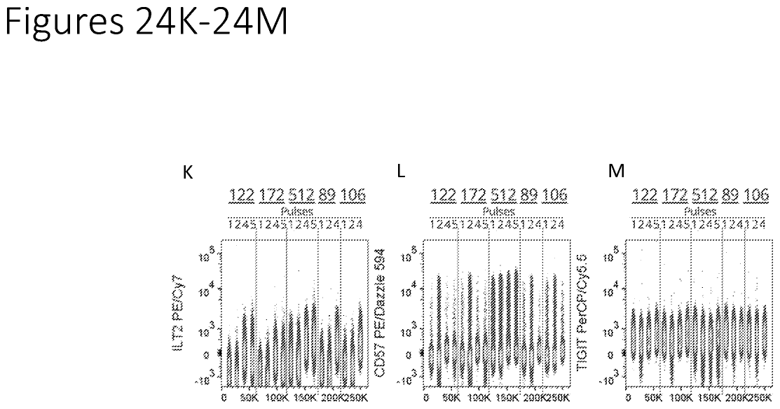

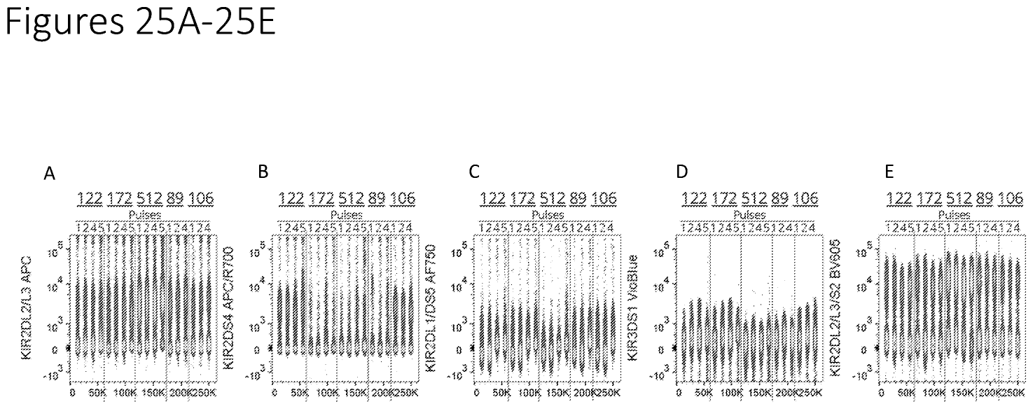

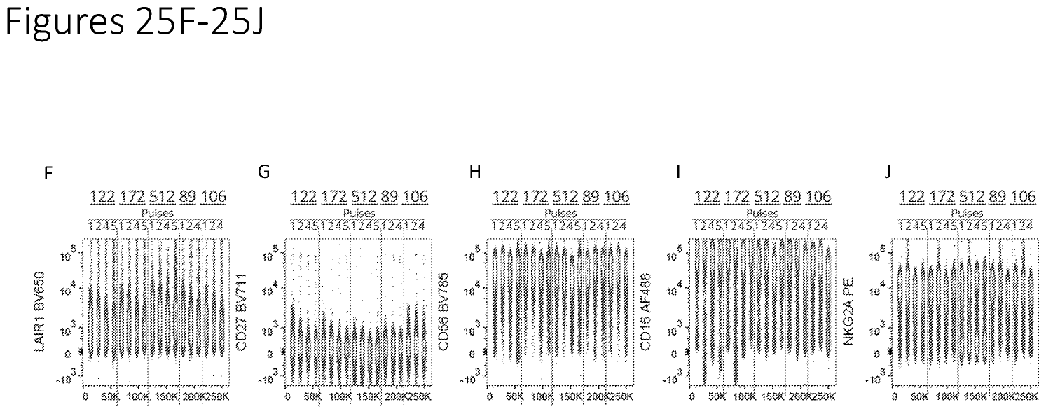

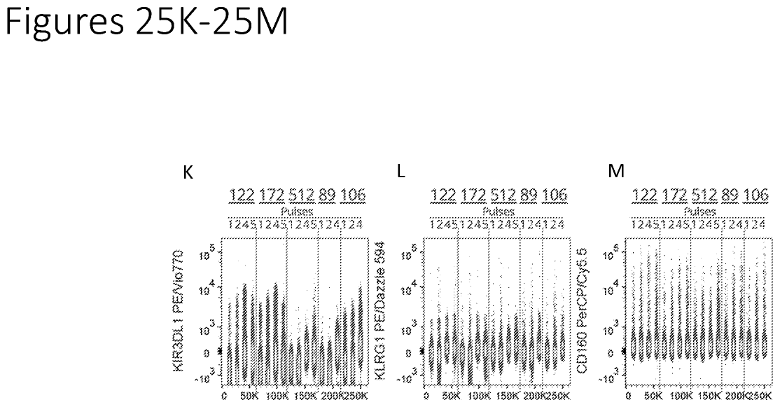

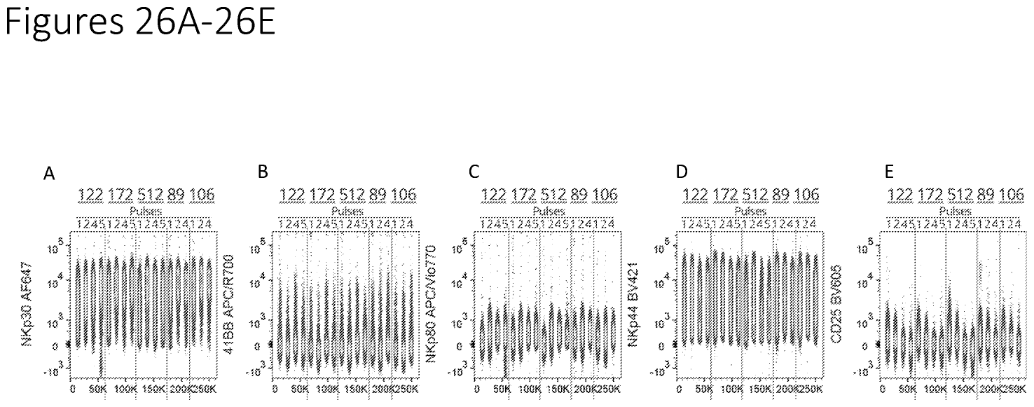

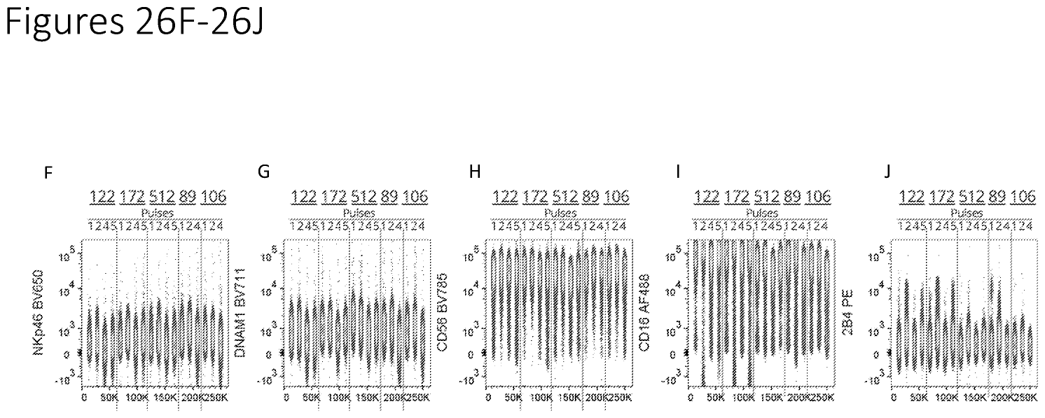

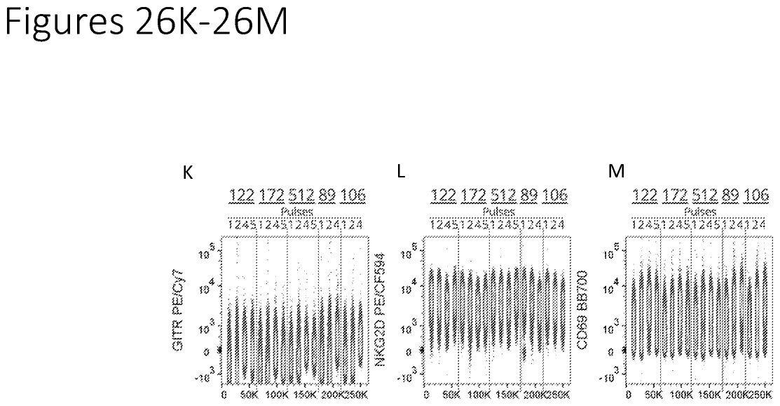

In order to attempt to correlate the degree of expansion with characteristics of the cells from the CB or PB donors, expression of various markers were assessed during the expansion process. These data are shown in FIGS. 24A-26M. Expression was measured at Days 14, 28, 56 and 70. The first panel of markers evaluated (shown in FIGS. 24A-24M) included NKG2C, CD39, TIM-3, OX40L, CD62L, LAG3, PD1, CD56, CD16, NKG2A, ILT2, CD57, and TIGIT. The second panel of markers evaluated (shown in FIGS. 25A-25M) included KIR2DL2/L3, KIR2DS4, KIR2DL1/DS5, KIR3DS1, KIR2DL2/L3/S2, LAIR1, CD27, CD56, CD16, NKG2A, KIR3DL1, KLRG1, and CD160. The third panel of markers evaluated (shown in FIGS. 26A-26M) included NKp30, $1BB, NKp80, NKp44, CD25, NKp46, DNAM1, CD56, CD16, 2B4, GITR, NKG2D, and CD69.

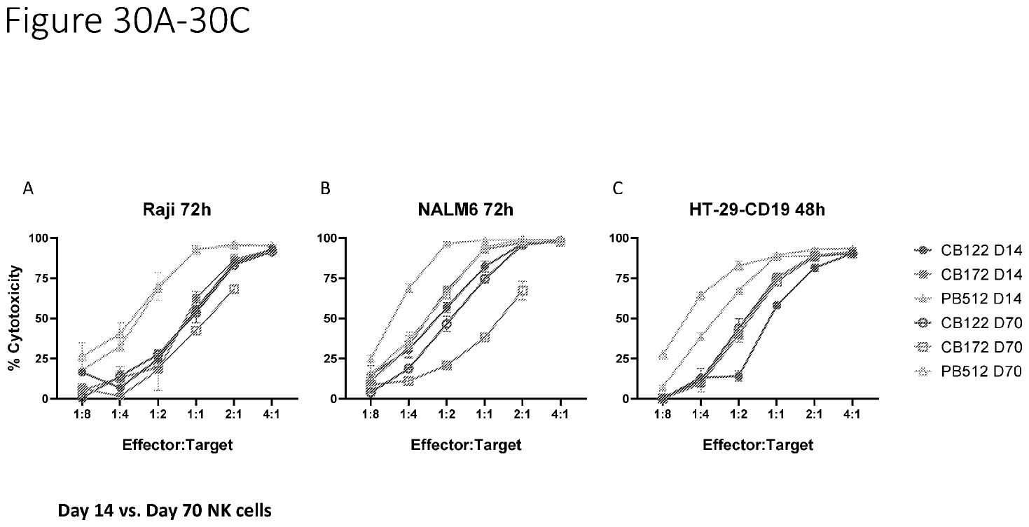

NKG2A is the first HLA class I specific inhibitory receptor to be expressed during NK cell differentiation. During an intermediate differentiation stage, NKG2A may be co-expressed with KIRs. At late stages, NKG2A expression is lost, whereas KIRs expression is maintained. In the first panel of markers assayed, most appeared to be relatively constant across the pulses and between CB and PB NK cells. However, as shown in FIG. 24J, in the PB and not the CB NK cells, NKG2A expression was lost by Day 70, correlating with a later NK differentiation stage. NKG2A is an inhibitory surface receptor on NK cells and reducing NKG2A expression may reduce an inhibitory signaling cascade and allow for the maintained anti-tumor potency as shown in FIGS. 30A-30C.

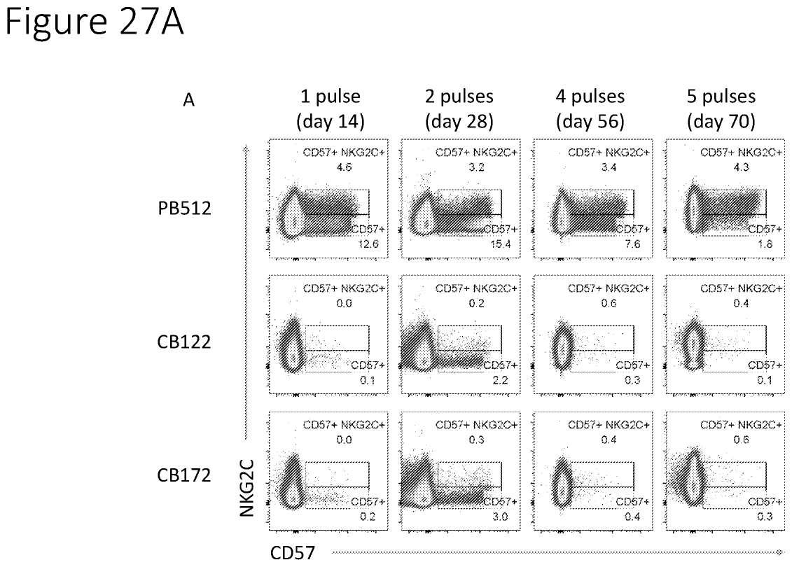

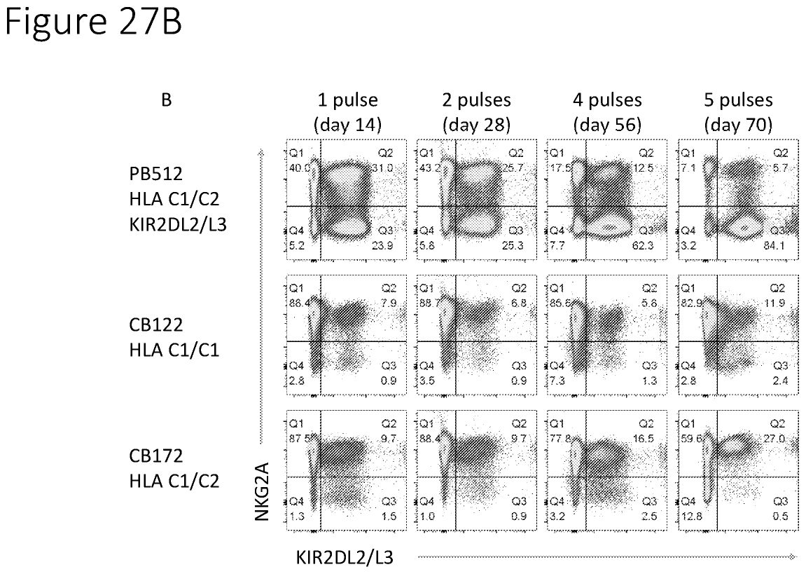

The second panel of markers, in accordance with disclosure herein related to various KIRs and their ratios being assessed to identify promising donors, evaluated several sets of KIR inhibitory receptors. As shown in FIG. 25A, the expression of the inhibitory KIR2DL2/L3 inhibitory receptor appeared to be elevated in PB NK cells as compared to CB NK cells, but relatively constant across the pulses during expansion, with a slight increase from 54% at Day 14 after 1 pulse, to 90% at Day 70 with 5 pulses (Percentages shown in FIG. 27B).

In marker panel 3, FIG. 26C, most of the markers remain consistent except for an increase in CD69 expression. As shown in FIG. 26M, CD69 expression appears to be elevated with pulse number. CD69 expression is increased after NK cells are stimulation with IL2, so this increase is not unexpected. CD69 expression increase may also reflect its function as a costimulatory molecule during expansion or sustaining NK cell activation (as has been seen in T cells).

Taken together, these marker screens indicate that the final expanded CB NK cells were CD57-KIRI^{lo/-} and NKG2A⁺, consistent with an immature phenotype. The final expanded PB NK cells were educated, at more than 80% NKG2A⁻KIR⁺. Moreover, the data from these expression panels suggest that the expansion process may not only increase the cell number, but also alter the expanded cells activation and/or persistence.

To further investigate the expansion and activation capacity of CB NK and PB NK cells, more specific screening was performed. As shown in FIG. 27A, expression of the activating NKG2C receptor and a marker of NK cell maturation, CD57, was evaluated. In general, increased expression of NKG2C and CD57 would be indicative of differentiation of NK cells (more expression means more differentiated), eventually taking on an adaptive-like phenotype. Interestingly, the two CB NK cell samples studied expressed very little CD57 at any time point during expansion. Additionally, the PB NK cell populations expressing little to no NKG2C appears to also show a reduction in percentage over time (dropping from ˜13% to about 2% over the 70-day expansion process). CD57 is typically used to identify terminally differentiated cells with reduced proliferative capacity, so the reduced expression of CD57 may represent a reversal of that status to a state with additional proliferation potential.

As discussed herein, the KIR profile of a given donor cell may impact the overall expansion and/or activity capacity of NK cells expanded from that donor. NK cells that are “educated” (prior interaction of inhibitory KIRs with MHC ligands) have been shown to be hyperresponsive to stimulation. KIR educated cells must also have expression of the KIRs on their cell surface. Given that CB cells are relatively young, their KIR expression is lower than that of PB cells. The expression of NKG2A (inhibitory) was evaluated along with KIR2DL2/3 expression. FIG. 27B shows the resultant data. This Figure shows demonstrates that there is a preferential expansion of KIR educated PB NK cells which is also associated with a reduced level of NKG2A expression. As seen in FIG. 27B, at day 14 the percentage of cells expressing low KIR2DL2/3 and high NKG2A was about 40% (see Q1) while those expressing low NKG2A and higher KIR2DL2/3 was about 24% (see Q4). The expansion process resulted in a “shift” of cells from Q1 to Q4 over the 70 days, as shown in the decrease to ˜7% cells in Q1 at Day 70 (high NKG2A/low KIR) and the increase to ˜85% of cells in Q4 at Day 70 (low NKG2A/high KIR). These data suggest that, in several embodiments, use of a KIR educated (e.g., peripheral blood) starting NK cell population may enhance the expansion potential.

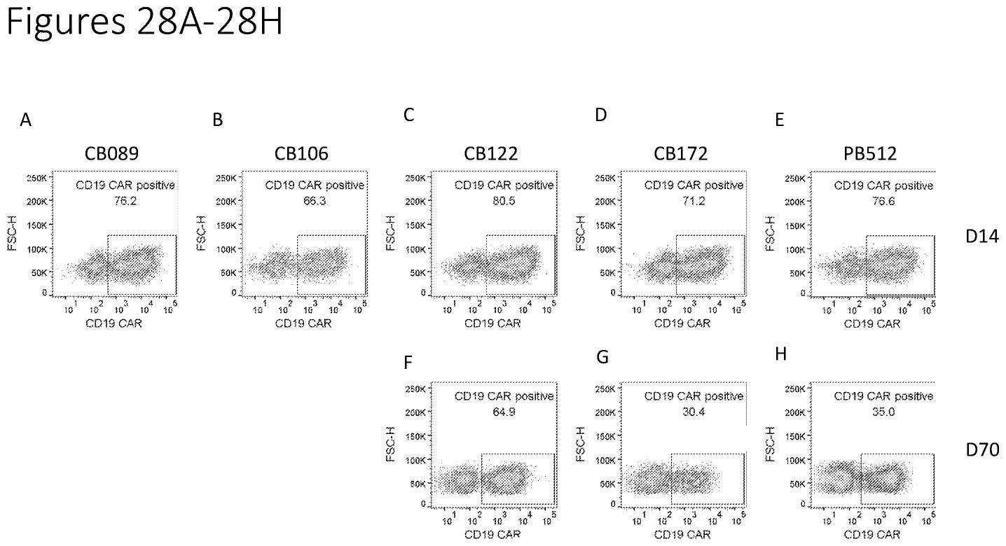

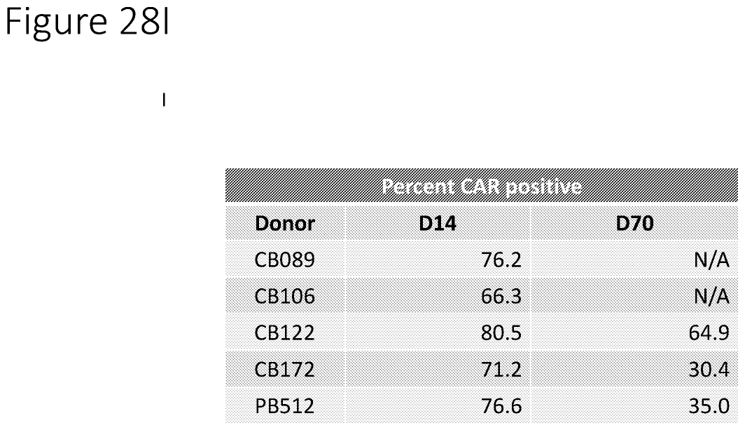

While robust expansion of NK cells using the methods disclosed herein is an important aim, maintaining the expression of CAR constructs that those cells have been engineered to express, such that the resulting expanded population maintains the desired engineered-in cytotoxicity, is also a central objective. As mentioned above, PB NK and CB NK cells were transduced with, as a non-limiting example, a CD19-directed CAR. In this example, opposed to Example 3 above, in which the NK cells were engineered to express the CAR and also edited to reduce expression of CISH, the PB NK and CB NK cells were not gene edited. FIGS. 28A-28H show CD19 expression data at Day 14 of expansion (top row) and at Day 70 (bottom row). It is notable in this evaluation that both CB NK cells (compare 28C to 28F and 28D to 28G) and PB NK cells (compare 28E to 28H) exhibited decreases in the expression of the CD19 CAR over the course of expansion. FIG. 281 tabulates the expression data. As compared to, for example, FIG. 17A, in which PB NK cells edited for CISH maintained CD19 CAR expression, here, the absence of CISH led to a reduction in expression of the CAR. As will be discussed below, the reduced expression still allowed for cytotoxicity, however, in several embodiments, dis-inhibiting the positive impact of mbIL15 expression (which is encoded by, though separately expressed, the polynucleotide encoding the CD19 CAR construct) by genetically reducing levels of CISH, results in maintained (or less reduction) in CD19 CAR expression. In several embodiments, such edits result in further enhanced cytotoxicity.

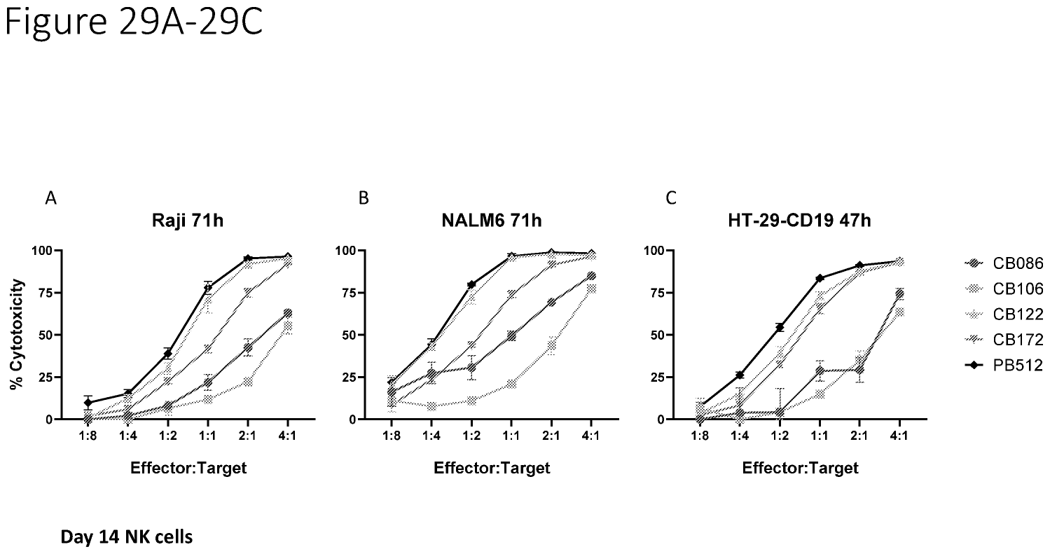

FIGS. 29A-29C show cytotoxicity data of PB NK and CB NK cells (while still expressing higher levels of the CD19 CAR (as shown in FIG. 28, top row) at Day 14 of expansion. Cytotoxicity was evaluated against Raji cells (Burkitt lymphoma), NALM6 cells (B cell precursor leukemia), and HT-29-CD19 (a colorectal adenocarcinoma engineered to ectopically express CD19). Percent cytotoxicity was calculated based on Incucyte images (representing fluorescence intensity of target tumor cells) collected at the indicated timepoint after co-culture and the indicated E:T ratio. As shown in the figures, at least with these three non-limiting embodiments of target tumor cell lines, the PB NK cells expressing the CD19 CAR appeared to have a greater cytotoxicity against the target cells in comparison to the CB NK cells, though the CB NK cells from donor 122 (upright triangle) performed quite similarly against both the RAJI and NALM6 cells lines. CB donor 172 (inverted triangles) showed reduced cytotoxicity and CB donors 086 and 106 showed the least cytotoxicity, though still able to achieve ˜60-70% cytotoxicity at a 4:1 E:T ratio. It is noted that these two donors also did not complete the full 70 day expansion at therefore may represent NK cell samples that are otherwise less robust (in terms of overall cell health, impacting both expansion capacity and cytotoxicity) since they did express similar levels of the CAR (see FIG. 281).

Despite the reduced expression of the CD19 CAR observed at 70 days discussed above, the expanded NK cells still retained substantial cytotoxicity against target tumor cells, as shown in FIGS. 30A-30C. Cytotoxicity was evaluated against the same three cell lines as in FIGS. 29A-29C but comparing Day 14 cells versus Day 70. Day 14 data is shown with filled shapes and Day 70 is shown in open shapes. With Raji cells as the target, PB NK cells (triangles) showed little to no difference in terms of cytotoxicity generated by Day 14 versus Day 70 cells. Similar results are shown for CB NK cells from donor 122 (circles), though some decreased cytotoxicity was observed for CB NK cells from donor 172 (squares). Cytotoxicity was somewhat more distinct for Day 14 versus Day 70 cells when targeting NALM6 cells. With each sample, the Day 14 cells appeared to show modestly higher cytotoxicity, although again, it should be noted that at a 1:1 E:T ratio 5 of 6 of the experimental groups achieved or exceeded ˜75% cytotoxicity. In contrast, against the ectopic CD19 expressing HT-29-CD19 cells, the PB NK cells and CB NK cells from donor 122 appeared more potent after 70 days, despite the reduced CD19 CAR expression. CB NK cells from donor 172 performed substantially similar to one another. Notable again is, at 2:1 E:T, all cell samples achieved or exceeded 75% cytotoxicity (5 of 6 met this performance level at 1:1 E:T). In several embodiments, the positive activating and persistence effects of the expansion processes disclosed herein are sufficient to offset a reduced expression of the cancer targeting CAR that may occur, though it can be obviated, in several embodiments, by gene editing of, for example CISH. Taken together, these data demonstrate that even profoundly expanded cell populations retain significant cytotoxicity against target cells. With the PB NK cells, potency is maintained even after a 250 billion-fold expansion. Thus, according to several embodiments disclosed herein, expansion of immune cells, such as NK cells, that are engineered to express a tumor targeting CAR (and/or edited at one or more gene targets) yields substantial increases in cell populations, and maintenance of significant cytotoxic potential against target tumor cells.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering a population of expanded NK cells” includes “instructing the administration of a population of expanded NK cells.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “90%” includes “90%.” In some embodiments, at sequence having at least 95% sequence identity with a reference sequence includes sequences having 96%, 97%, 98%, 99%, or 100% identical to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

Articles such as “a”, “an”, “the” and the like, may mean one or more than one unless indicated to the contrary or otherwise evident from the context. The phrase “and/or” as used herein in the specification and in the claims, should be understood to mean “either or both” of the elements so conjoined. Multiple elements listed with “and/or” should be construed in the same fashion, i.e., “one or more” of the elements so conjoined. Other elements may optionally be present other than the elements specifically identified by the “and/or” clause. As used herein in the specification and in the claims, “or” should be understood to have the same meaning as “and/or” as defined above. For example, when used in a list of elements, “or” or “and/or” shall be interpreted as being inclusive, i.e., the inclusion of at least one, but optionally more than one, of list of elements, and, optionally, additional unlisted elements. Only terms clearly indicative to the contrary, such as “only one of” or “exactly one of” will refer to the inclusion of exactly one element of a number or list of elements. Thus claims that include “or” between one or more members of a group are considered satisfied if one, more than one, or all of the group members are present, employed in, or otherwise relevant to a given product or process unless indicated to the contrary. Embodiments are provided in which exactly one member of the group is present, employed in, or otherwise relevant to a given product or process. Embodiments are provided in which more than one, or all of the group members are present, employed in, or otherwise relevant to a given product or process. Any one or more claims may be amended to explicitly exclude any embodiment, aspect, feature, element, or characteristic, or any combination thereof. Any one or more claims may be amended to exclude any agent, composition, amount, dose, administration route, cell type, target, cellular marker, antigen, targeting moiety, or combination thereof.

In several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

Any titles or subheadings used herein are for organization purposes and should not be used to limit the scope of embodiments disclosed herein.

1-59. (canceled)

60. A method for enhancing the expansion of natural killer cells, comprising:
co-culturing a population of natural killer (NK) cells with a first feeder cell population thereby generating a first co-cultured NK cell population, wherein the first feeder cell population comprises cells engineered to express 4-1BBL and a membrane-bound interleukin-15 (mbIL15);
co-culturing the first co-cultured NK cell population, with a second feeder cell population, thereby generating a second co-cultured NK cell population;
co-culturing, the second co-cultured NK cell population with a third feeder cell population, thereby generating a third co-cultured NK cell population;
co-culturing the third co-cultured NK cell population with a fourth feeder cell population, thereby generating a fourth co-cultured NK cell population; and
co-culturing the fourth co-cultured NK cell population with a fifth feeder cell population, thereby generating a fifth co-cultured NK cell population, wherein the culture media for each co-culturing comprises a soluble IL12 and a soluble IL18, and wherein the fifth co-cultured population of NK cells is an expanded population of NK cells as compared to the first co-cultured NK cell population.

61. The method of claim 60, wherein the ratio of NK cells to feeder cells at each co-culturing ranges from 1:2 to 1:10.

62. The method of claim 60, wherein the ratio of NK cells to feeder cells at each co-culturing ranges from 1:3 to 1:5.

63. The method of claim 60, wherein the IL12 present in the culture media is at a concentration ranging from 0.01 ng/ml to 10 ng/mL.

64. The method of claim 60, wherein the IL18 present in the culture media is at a concentration ranging from 10 ng/ml to 30 ng/mL.

65. The method of claim 60, wherein the culture media further comprises a soluble IL2 for at least one co-culturing, wherein the soluble IL2 is present in the culture media at a concentration ranging from 25 to 50 units/mL and wherein the IL2 is present in the culture media for at least the first and the fifth co-culturing.

66. The method of claim 60, wherein the NK cells are frozen at least two times between the first and the fifth co-culturing.

67. The method of claim 60, further comprising modifying the NK cells to reduce or eliminate expression of CISH.

68. The method of claim 60, further comprising modifying the NK cells to express a chimeric antigen receptor that is directed against a tumor target selected from a ligand for the NKG2D receptor, CD19, CD70, BCMA, or CD38.

69. The method of claim 60, wherein the NK cells produced by the method express aKIR and iKIR receptors and wherein the ratio of aKIR to iKIR expression of the NK cells prior to expansion was at least about 3.

70. A population of expanded Natural Killer (NK) cells, which have been modified to reduce or eliminate expression of CISH, wherein the population of expanded NK cells express aKIR and iKIR receptors and, wherein the ratio of aKIR to iKIR expression prior to expansion was at least about 3.

71. The population of expanded NK cells of claim 70, wherein the population of expanded NK cells comprise a CAR, which targets a ligand of the NKG2D receptor, CD19, CD70, BCMA, or CD38.

72. The population of expanded NK cells of claim 70, wherein the population of expanded NK cells are generated by:
co-culturing a population of NK cells with a first feeder cell population thereby generating a first co-cultured NK cell population, wherein the first feeder cell population comprises cells engineered to express 4-1BBL and a membrane-bound interleukin-15 (mbIL15);
co-culturing the first co-cultured NK cell population, with a second feeder cell population, thereby generating a second co-cultured NK cell population;
co-culturing, the second co-cultured NK cell population with a third feeder cell population, thereby generating a third co-cultured NK cell population;
co-culturing the third co-cultured NK cell population with a fourth feeder cell population, thereby generating a fourth co-cultured NK cell population; and
co-culturing the fourth co-cultured NK cell population with a fifth feeder cell population, thereby generating a fifth co-cultured NK cell population,
wherein the culture media for each co-culturing comprises a soluble IL12 and a soluble IL18.

73. The population of expanded NK cells of claim 60, wherein the NK cells are obtained from a peripheral blood sample.

74. The population of expanded NK cells of claim 60, wherein the NK cells are obtained from a cord blood sample.

75. A method for treating or inhibiting a cancer in a subject, comprising:
administering to the subject an expanded population of Natural Killer (NK) cells, wherein the expanded population of NK cells express a chimeric antigen receptor, which targets a ligand of the NKG2D receptor, CD19, CD70, BCMA, or CD38, wherein the expanded population of NK cells express a reduced amount of CISH as compared to a native NK cell or express aKIR and iKIR receptors, wherein the ratio of aKIR to iKIR expression of the expanded population of NK cells prior to expansion was at least about 3.

76. The method of claim 75, wherein the expanded population of NK cells are generated by:
co-culturing a population of NK cells with a first feeder cell population thereby generating a first co-cultured NK cell population, wherein the first feeder cell population comprises cells engineered to express 4-1BBL and a membrane-bound interleukin-15 (mbIL15),
co-culturing the first co-cultured NK cell population, with a second feeder cell population, thereby generating a second co-cultured NK cell population;
co-culturing, the second co-cultured NK cell population with a third feeder cell population, thereby generating a third co-cultured NK cell population;
co-culturing the third co-cultured NK cell population with a fourth feeder cell population, thereby generating a fourth co-cultured NK cell population; and
co-culturing the fourth co-cultured NK cell population with a fifth feeder cell population, thereby generating a fifth co-cultured NK cell population, wherein the culture media for each co-culturing comprises a soluble IL12 and a soluble IL18.

77. A method for identifying a preferred donor of immune cells for immunotherapy, comprising:
detecting an expression level of at least one activating Killer Cell Ig-Like Receptor (aKIR) in a blood sample comprising immune cells;
detecting an expression level of at least one inhibitory Killer Cell Ig-Like Receptor (iKIR);
calculating a ratio of the expression level of the at least one aKIR and the at least one iKIR; and
categorizing the candidate donor as a preferred donor if the ratio of aKIR to iKIR exceeds a threshold value, wherein the threshold value is above about 3.

78. The method of claim 77, further comprising assessing the ability of the immune cells from the candidate donor to be expanded in culture prior to said categorizing.

79. The method of claim 77, further comprising assessing the ability of the immune cells from the candidate donor to exert cytotoxic effects on a target tumor cell prior to said categorizing.

80. The method of claim 77, further comprising assessing the cytomegalovirus (CMV) status of the immune cells from the candidate donor prior to said categorizing.

81. The method of claim 77, further comprising detecting the degree of Human Leukocyte Antigen (HLA) mismatch between immune cells from the candidate donor and a target tumor cell by determining the number of iKIR triggered by tumor HLA.


SEQUENCE LISTING (70 sequences)

SEQ ID NO: 1 - DNA, 1797 bp, synthetic construct
  misc_feature 1..1797: note = Artificial Sequence
  misc_feature 1..1797: note = NK45-4 DNA [with mbIL15]
  source 1..1797: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 2 - protein, 599 aa, synthetic construct
  REGION 1..599: note = Artificial Sequence
  REGION 1..599: note = MISC_FEATURE - NK45-5 AA [w/ mbIL15]
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 3 - DNA, 2150 bp, synthetic construct
  misc_feature 1..2150: note = Artificial Sequence
  misc_feature 1..2150: note = DNA NK19H-No Flag-1
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 4 - protein, 706 aa, synthetic construct
  REGION 1..706: note = Artificial Sequence
  REGION 1..706: note = MISC_FEATURE - AA NK19H-No Flag-1
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 5 - DNA, 2150 bp, synthetic construct
  misc_feature 1..2150: note = Artificial Sequence
  misc_feature 1..2150: note = DNA NK19H-No Flag-2
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 6 - protein, 706 aa, synthetic construct
  REGION 1..706: note = Artificial Sequence
  REGION 1..706: note = MISC_FEATURE - AA NK19H-No Flag-2
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 7 - DNA, 2150 bp, synthetic construct
  misc_feature 1..2150: note = Artificial Sequence
  misc_feature 1..2150: note = DNA NK19H-No Flag-3
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattataccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 8 - protein, 706 aa, synthetic construct
  REGION 1..706: note = Artificial Sequence
  REGION 1..706: note = MISC_FEATURE - AA NK19H-No Flag-3
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 9 - DNA, 2150 bp, synthetic construct
  misc_feature 1..2150: note = Artificial Sequence
  misc_feature 1..2150: note = DNA NK19H-No Flag-4
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggctgggcgtgatttggggcagcgaaaccacctattataacagcgcgctgaaaagccgcctgaccattagcaaagataacagcaaaagccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 10 - protein, 706 aa, synthetic construct
  REGION 1..706: note = Artificial Sequence
  REGION 1..706: note = MISC_FEATURE - AA NK19H-No Flag-4
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 11 - DNA, 2153 bp, synthetic construct
  misc_feature 1..2153: note = Artificial Sequence
  misc_feature 1..2153: note = DNA NK19H-No Flag-5
  source 1..2153: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggagtcaggacctggcctggtgaaaccctcacagactctgtccctgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcagatctagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 12 - protein, 707 aa, synthetic construct
  REGION 1..707: note = Artificial Sequence
  REGION 1..707: note = MISC_FEATURE - AA NK19H-No Flag-5
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 13 - DNA, 2150 bp, synthetic construct
  misc_feature 1..2150: note = Artificial Sequence
  misc_feature 1..2150: note = DNA NK19H-No Flag-6
  source 1..2150: mol_type = other DNA
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccagacggaactgttaaactcctgatctaccatacatcaagattacactcaggagtcccatcaaggttcagtggcagtgggtctggaacagattacaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccctgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 14 - protein, 706 aa, synthetic construct
  REGION 1..706: note = Artificial Sequence
  REGION 1..706: note = MISC_FEATURE - AA NK19H-No Flag-6
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 15 - DNA, 2150 bp, synthetic construct
  misc_feature 1..2150: note = Artificial Sequence
  misc_feature 1..2150: note = DNA NK19H-No Flag-7
  source 1..2150: mol_type = other DNA
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccaggtggaactgttaaactcctgatctaccatacatcaagattacactcaggagtcccatcaaggttcagtggcagtgggtctggaacagatttcaccctcaccattagcagcctgcaaccggaagatattgccacttactactgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 16 - protein, 706 aa, synthetic construct
  REGION 1..706: note = Artificial Sequence
  REGION 1..706: note = MISC_FEATURE - AA NK19H-No Flag-7
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 17 - DNA, 2150 bp, synthetic construct
  misc_feature 1..2150: note = Artificial Sequence
  misc_feature 1..2150: note = DNA NK19H-No Flag-8
  source 1..2150: mol_type = other DNA
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccaggtggaactgttaaactcctgatctaccatacatcaagattacactcaggagtcccatcaaggttcagtggcagtgggtctggaacagatttcaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 18 - protein, 706 aa, synthetic construct
  REGION 1..706: note = Artificial Sequence
  REGION 1..706: note = MISC_FEATURE - AA NK19H-No Flag-8
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 19 - DNA, 2150 bp, synthetic construct
  misc_feature 1..2150: note = Artificial Sequence
  misc_feature 1..2150: note = DNA NK19H-No Flag-9
  source 1..2150: mol_type = other DNA
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccagacggaactgttaaactcctgatctaccatacatcaagattacactcaggagtcccatcaaggttcagtggcagtgggtctggaacagattacaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 20 - protein, 706 aa, synthetic construct
  REGION 1..706: note = Artificial Sequence
  REGION 1..706: note = MISC_FEATURE - AA NK19H-No Flag-9
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 21 - DNA, 2150 bp, synthetic construct
  misc_feature 1..2150: note = Artificial Sequence
  misc_feature 1..2150: note = DNA NK19H-No Flag-10
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 22 - protein, 706 aa, synthetic construct
  REGION 1..706: note = Artificial Sequence
  REGION 1..706: note = MISC_FEATURE - AA NK19H-No Flag-10
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 23 - DNA, 2150 bp, synthetic construct
  misc_feature 1..2150: note = Artificial Sequence
  misc_feature 1..2150: note = DNA NK19H-No Flag-11
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 24 - protein, 706 aa, synthetic construct
  REGION 1..706: note = Artificial Sequence
  REGION 1..706: note = MISC_FEATURE - AA NK19H-No Flag-11
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 25 - DNA, 2150 bp, synthetic construct
  misc_feature 1..2150: note = Artificial Sequence
  misc_feature 1..2150: note = DNA NK19H-No Flag-12
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattataccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 26 - protein, 706 aa, synthetic construct
  REGION 1..706: note = Artificial Sequence
  REGION 1..706: note = MISC_FEATURE - AA NK19H-No Flag-12
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 27 - DNA, 2210 bp, synthetic construct
  misc_feature 1..2210: note = Artificial Sequence
  misc_feature 1..2210: note = DNA NK19-1
  source 1..2210: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 28 - protein, 724 aa, synthetic construct
  REGION 1..724: note = Artificial Sequence
  REGION 1..724: note = MISC_FEATURE - AA NK19-1
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 29 - DNA, 20 bp, synthetic construct
  misc_feature 1..20: note = Artificial Sequence
  misc_feature 1..20: note = CISH gRNA 1
  source 1..20: mol_type = other DNA
ctcaccagattcccgaaggt

SEQ ID NO: 30 - DNA, 20 bp, synthetic construct
  misc_feature 1..20: note = Artificial Sequence
  misc_feature 1..20: note = CISH gRNA 2
  source 1..20: mol_type = other DNA
ccgccttgtcatcaaccgtc

SEQ ID NO: 31 - DNA, 20 bp, synthetic construct
  misc_feature 1..20: note = Artificial Sequence
  misc_feature 1..20: note = CISH gRNA 3
  source 1..20: mol_type = other DNA
tctgcgttcaggggtaagcg

SEQ ID NO: 32 - DNA, 20 bp, synthetic construct
  misc_feature 1..20: note = Artificial Sequence
  misc_feature 1..20: note = CISH gRNA 4
  source 1..20: mol_type = other DNA
gcgcttacccctgaacgcag

SEQ ID NO: 33 - DNA, 20 bp, synthetic construct
  misc_feature 1..20: note = Artificial Sequence
  misc_feature 1..20: note = CISH gRNA 5
  source 1..20: mol_type = other DNA
cgcagaggaccatgtccccg

SEQ ID NO: 34 - DNA, 20 bp, synthetic construct
  misc_feature 1..20: note = Artificial Sequence
  misc_feature 1..20: note = TGFBR2 gRNA 1
  source 1..20: mol_type = other DNA
cccctaccatgactttattc

SEQ ID NO: 35 - DNA, 20 bp, synthetic construct
  misc_feature 1..20: note = Artificial Sequence
  misc_feature 1..20: note = TGFBR2 gRNA 2
  source 1..20: mol_type = other DNA
attgcactcatcagagctac

SEQ ID NO: 36 - DNA, 20 bp, synthetic construct
  misc_feature 1..20: note = Artificial Sequence
  misc_feature 1..20: note = TGFBR2 gRNA 3
  source 1..20: mol_type = other DNA
agtcatggtaggggagcttg

SEQ ID NO: 37 - DNA, 20 bp, synthetic construct
  misc_feature 1..20: note = Artificial Sequence
  misc_feature 1..20: note = TGFBR2 gRNA 4
  source 1..20: mol_type = other DNA
tgctggcgatacgcgtccac

SEQ ID NO: 38 - DNA, 20 bp, synthetic construct
  misc_feature 1..20: note = Artificial Sequence
  source 1..20: mol_type = other DNA
gtgagcaatcccccgggcga

SEQ ID NO: 39 - DNA, 20 bp, synthetic construct
  misc_feature 1..20: note = Artificial Sequence
  misc_feature 1..20: note = TGFBR2 gRNA 6
  source 1..20: mol_type = other DNA
aacgtgcggtgggatcgtgc

SEQ ID NO: 40 - DNA, 1122 bp, synthetic construct
  misc_feature 1..1122: note = Artificial Sequence
  misc_feature 1..1122: note = NKG2D-OX40-CD3Z [no mbIL15]
  source 1..1122: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 41 - protein, 374 aa, synthetic construct
  REGION 1..374: note = Artificial Sequence
  REGION 1..374: note = MISC_FEATURE - NKG2D-OX40-CD3z [no mbIL15]
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - DNA, 2181 bp, synthetic construct
  misc_feature 1..2181: note = Artificial Sequence
  misc_feature 1..2181: note = NK77.71_CD70_DB04_D02 with mbIL15
  source 1..2181: mol_type = other DNA
atggccttaccagtgaccgccttgctcctgccgctggccttgctgctccacgccgccaggccggactacaaagacgatgacgataaaggcggtggtggctctggtggtggcggatcccaggtgcagctggtgcagtctggggctgaggtgaagaagcctggggcctcagtgaaggtttcctgcaaggcatctggatacaccttcaccagctacgacatcaattgggtgcgacaggcccctggacaagggcttgagtggatgggagtcatcagccctagtggtggtggaacaagctacgcacagaagttccagggcagagtcaccatgaccagggacacgtccacgagcacagtctacatggagctgagcagcctgagatctgaggacacggccgtgtattactgtgctagatactacggcggcaaccccgaagggtggtacttcgatctctggggccgtggcaccctggtcactgtctcctcaggtggaggcggttcaggcggaggtggctctggcggtggcggatcggacatccagatgacccagtctccatcctccctgtctgcatctgtaggagacagagtcaccatcacttgccgggcaagtcagagcattggaaattggttagcctggtatcagcagaaaccagggaaagcccctaagctcctgatctatgacgcatccgatttggaaaccggggtcccatcaaggttcagtggcagtggatctgggacagatttcactctcaccatcagcagtctgcaacctgaagattttgcaacttactactgtcaacagagttacactaccccttggacgttcggccaagggaccaaggtggaaatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 43 - protein, 501 aa, synthetic construct
  REGION 1..501: note = Artificial Sequence
  REGION 1..501: note = MISC_FEATURE - NK77.71_CD70_DB04_D02 CAR only
MALPVTALLLPLALLLHAARPDYKDDDDKGGGGSGGGGSQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 2181 bp, synthetic construct
  misc_feature 1..2181: note = Artificial Sequence
  misc_feature 1..2181: note = NK77.17_CD70_DB02_B06 with mbIL15
  source 1..2181: mol_type = other DNA
atggccttaccagtgaccgccttgctcctgccgctggccttgctgctccacgccgccaggccggactacaaagacgatgacgataaaggcggtggtggctctggtggtggcggatcccaggtgcagctggtgcagtctggggctgaggtgaagaagcctggggcctcagtgaaggtttcctgcaaggcatctggatacaccttcaccgattactatatgcactgggtgcgacaggcccctggacaagggcttgagtggatgggaataatcaaccctaatagtggtggaacaaattacgcacagaagttccagggcagagtcaccatgaccagggacacgtccacgagcacagtctacatggagctgagcagcctgagatctgaggacacggccgtgtattactgtgctagagattttagtgtgtatgctgctgcagatggcatggatgtctggggccaagggacattggtcaccgtctcttcaggtggaggcggttcaggcggaggtggctctggcggtggcggatcggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgacgcatccaatttggccaccggggtcccatcaaggttcagtggcagtggatctgggacagatttcactctcaccatcagcagtctgcaacctgaagattttgcaacttactactgtcaacagagtgatagttcacctctcactttcggccaagggaccaaggtggagatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 45 - protein, 501 aa, synthetic construct
  REGION 1..501: note = Artificial Sequence
  REGION 1..501: note = MISC_FEATURE - NK77.17_CD70_DB02_B06 CAR only
MALPVTALLLPLALLLHAARPDYKDDDDKGGGGSGGGGSQVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSSGGGGSGGGGSGGGGSDIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - DNA, 2172 bp, synthetic construct
  misc_feature 1..2172: note = Artificial Sequence
  misc_feature 1..2172: note = NK77.58_CD70_DB03_H08 with mbIL15
  source 1..2172: mol_type = other DNA
atggccttaccagtgaccgccttgctcctgccgctggccttgctgctccacgccgccaggccggactacaaagacgatgacgataaaggcggtggtggctctggtggtggcggatcccaggtgcagctggtgcagtctggggctgaggtgaagaagcctggggcctcagtgaaggtttcctgcaaggcatctggatacaccttcaccagctacgacattaattgggtgcgacaggcccctggacaagggcttgagtggatgggagtgatcaaccctagtggtggaggtacaagctacgcacagaagttccagggcagagtcaccatgaccagggacacgtccacgagcacagtctacatggagctgagcagcctgagatctgaggacacggccgtgtattactgtgctagatattctggctatgattgggtgtttgactactggggccaagggaccctggtcaccgtctcctcaggtggaggcggttcaggcggaggtggctctggcggtggcggatcggacatccagatgacccagtctccatcctccctgtctgcatctgtaggagacagagtcaccatcacttgccgggcaagtcagggcattggcagctggttagcttggtatcagcagaaaccagggaaagcccctaagctcctgatctatgctgcatccacattgcaaagtggggtcccatcaaggttcagtggcagtggatctgggacagatttcactctcaccatcagcagtctgcaacctgaagattttgcaacttactactgtcaacagagttacagtaccccttggactttcggccctgggaccaaagtggatatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 47 - protein, 498 aa, synthetic construct
  REGION 1..498: note = Artificial Sequence
  REGION 1..498: note = MISC_FEATURE - NK77.58_CD70_DB03_H08 CAR only
MALPVTALLLPLALLLHAARPDYKDDDDKGGGGSGGGGSQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 481 aa, synthetic construct
  REGION 1..481: note = Artificial Sequence
  REGION 1..481: note = MISC_FEATURE - AA NK19H-No Flag-1 CAR only
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 481 aa, synthetic construct
  REGION 1..481: note = Artificial Sequence
  REGION 1..481: note = MISC_FEATURE - AA NK19H-No Flag-2 CAR only
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 481 aa, synthetic construct
  REGION 1..481: note = Artificial Sequence
  REGION 1..481: note = MISC_FEATURE - AA NK19H-No Flag-3 CAR only
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 481 aa, synthetic construct
  REGION 1..481: note = Artificial Sequence
  REGION 1..481: note = MISC_FEATURE - AA NK19H-No Flag-4 CAR only
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 482 aa, synthetic construct
  REGION 1..482: note = Artificial Sequence
  REGION 1..482: note = MISC_FEATURE - AA NK19H-No Flag-5 CAR only
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 481 aa, synthetic construct
  REGION 1..481: note = Artificial Sequence
  REGION 1..481: note = MISC_FEATURE - AA NK19H-No Flag-6 CAR only
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 481 aa, synthetic construct
  REGION 1..481: note = Artificial Sequence
  REGION 1..481: note = MISC_FEATURE - AA NK19H-No Flag-7 CAR only
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 481 aa, synthetic construct
  REGION 1..481: note = Artificial Sequence
  REGION 1..481: note = MISC_FEATURE - AA NK19H-No Flag-8 CAR only
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 481 aa, synthetic construct
  REGION 1..481: note = Artificial Sequence
  REGION 1..481: note = MISC_FEATURE - AA NK19H-No Flag-9 CAR only
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 481 aa, synthetic construct
  REGION 1..481: note = Artificial Sequence
  REGION 1..481: note = MISC_FEATURE - AA NK19H-No Flag-10 CAR only
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 481 aa, synthetic construct
  REGION 1..481: note = Artificial Sequence
  REGION 1..481: note = MISC_FEATURE - AA NK19H-No Flag-11 CAR only
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 481 aa, synthetic construct
  REGION 1..481: note = Artificial Sequence
  REGION 1..481: note = MISC_FEATURE - AA NK19H-No Flag-12 CAR only
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - DNA, 612 bp, synthetic construct
  misc_feature 1..612: note = Artificial Sequence
  misc_feature 1..612: note = mbIL15 DNA
  source 1..612: mol_type = other DNA
atggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 61 - protein, 204 aa, synthetic construct
  REGION 1..204: note = Artificial Sequence
  REGION 1..204: note = MISC_FEATURE - mbIL15 AA
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 62 - DNA, 2127 bp, synthetic construct
  source 1..2127: mol_type = other DNA
atggccttaccagtgaccgccttgctcctgccgctggccttgctgctccacgccgccaggccgcaggtgcagctggtgcagtctggggctgaggtgaagaagcctggggcctcagtgaaggtttcctgcaaggcatctggatacaccttcaccgattactatatgcactgggtgcgacaggcccctggacaagggcttgagtggatgggaataatcaaccctaatagtggtggaacaaattacgcacagaagttccagggcagagtcaccatgaccagggacacgtccacgagcacagtctacatggagctgagcagcctgagatctgaggacacggccgtgtattactgtgctagagattttagtgtgtatgctgctgcagatggcatggatgtctggggccaagggacattggtcaccgtctcttcaggtggaggcggttcaggcggaggtggctctggcggtggcggatcggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgacgcatccaatttggccaccggggtcccatcaaggttcagtggcagtggatctgggacagatttcactctcaccatcagcagtctgcaacctgaagattttgcaacttactactgtcaacagagtgatagttcacctctcactttcggccaagggaccaaggtggagatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 63 - DNA, 2118 bp, synthetic construct
  source 1..2118: mol_type = other DNA
atggccttaccagtgaccgccttgctcctgccgctggccttgctgctccacgccgccaggccgcaggtgcagctggtgcagtctggggctgaggtgaagaagcctggggcctcagtgaaggtttcctgcaaggcatctggatacaccttcaccagctacgacattaattgggtgcgacaggcccctggacaagggcttgagtggatgggagtgatcaaccctagtggtggaggtacaagctacgcacagaagttccagggcagagtcaccatgaccagggacacgtccacgagcacagtctacatggagctgagcagcctgagatctgaggacacggccgtgtattactgtgctagatattctggctatgattgggtgtttgactactggggccaagggaccctggtcaccgtctcctcaggtggaggcggttcaggcggaggtggctctggcggtggcggatcggacatccagatgacccagtctccatcctccctgtctgcatctgtaggagacagagtcaccatcacttgccgggcaagtcagggcattggcagctggttagcttggtatcagcagaaaccagggaaagcccctaagctcctgatctatgctgcatccacattgcaaagtggggtcccatcaaggttcagtggcagtggatctgggacagatttcactctcaccatcagcagtctgcaacctgaagattttgcaacttactactgtcaacagagttacagtaccccttggactttcggccctgggaccaaagtggatatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 64 - DNA, 2127 bp, synthetic construct
  source 1..2127: mol_type = other DNA
atggccttaccagtgaccgccttgctcctgccgctggccttgctgctccacgccgccaggccgcaggtgcagctggtgcagtctggggctgaggtgaagaagcctggggcctcagtgaaggtttcctgcaaggcatctggatacaccttcaccagctacgacatcaattgggtgcgacaggcccctggacaagggcttgagtggatgggagtcatcagccctagtggtggtggaacaagctacgcacagaagttccagggcagagtcaccatgaccagggacacgtccacgagcacagtctacatggagctgagcagcctgagatctgaggacacggccgtgtattactgtgctagatactacggcggcaaccccgaagggtggtacttcgatctctggggccgtggcaccctggtcactgtctcctcaggtggaggcggttcaggcggaggtggctctggcggtggcggatcggacatccagatgacccagtctccatcctccctgtctgcatctgtaggagacagagtcaccatcacttgccgggcaagtcagagcattggaaattggttagcctggtatcagcagaaaccagggaaagcccctaagctcctgatctatgacgcatccgatttggaaaccggggtcccatcaaggttcagtggcagtggatctgggacagatttcactctcaccatcagcagtctgcaacctgaagattttgcaacttactactgtcaacagagttacactaccccttggacgttcggccaagggaccaaggtggaaatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 65 - protein, 708 aa, synthetic construct
MALPVTALLLPLALLLHAARPQVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSSGGGGSGGGGSGGGGSDIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 66 - protein, 705 aa, synthetic construct
MALPVTALLLPLALLLHAARPQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 67 - protein, 708 aa, synthetic construct
MALPVTALLLPLALLLHAARPQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 68 - protein, 483 aa, synthetic construct
MALPVTALLLPLALLLHAARPQVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSSGGGGSGGGGSGGGGSDIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 480 aa, synthetic construct
MALPVTALLLPLALLLHAARPQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 483 aa, synthetic construct
MALPVTALLLPLALLLHAARPQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR